(19) United States
(12) Patent Application Publication
(10) Pub. No.: US 2024/0190887 A1
(43) Pub. Date: Jun. 13, 2024

(54) COMPOUND USED AS SHP2 INHIBITOR AND USE THEREOF

(71) Applicants: TYK MEDICINES, INC., Huzhou, Zhejiang (CN); TYK MEDICINES (ZHENGZHOU), INC., Zhengzhou, Henan (CN)

(72) Inventors: Chengshan NIU, Zhengzhou, Henan (CN); Maolin ZHENG, Zhengzhou, Henan (CN); Apeng LIANG, Huzhou, Zhejiang (CN); Shaoqing CHEN, Huzhou, Zhejiang (CN); Jun LI, Huzhou, Zhejiang (CN); Yusheng WU, Huzhou, Zhejiang (CN)

(21) Appl. No.: 18/577,261

(22) PCT Filed: Jul. 7, 2022
(86) PCT No.: PCT/CN2022/104449

§ 371 Date: Jan. 7, 2024

(30) Foreign Application Priority Data
Jul. 7, 2021 (CN) 202110768781.9

(51) Int. Cl. C07D 491/107; A61K 31/4985; A61P 43/00

(52) U.S. Cl. CPC C07D 491/107; A61K 31/4985; A61P 43/00

(57) ABSTRACT

The present invention relates to a compound used as an SHP2 inhibitor and the use thereof. Specifically, the compound of the present invention has a structure as represented by formula I′, wherein the definitions of each group and each substituent are as described in the description. The compound has a high inhibitory effect on the activity of the SHP2 phosphatase, and can be used for preventing or treating SHP2-related diseases.

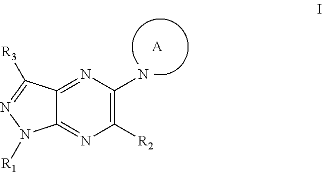

### TECHNICAL FIELD

The invention relates to the field of medical technology, in particular to compounds used as SHP2 inhibitors and their use in regulating SHP2 activity or treating SHP2 related diseases.

### BACKGROUND

SHP2 (encoded by the PTPN11 gene) is a member of the PTP family, which contains a conservative tyrosine phosphatase domain, two N-terminal SH2 domains, and a C-terminal tail. The two SH2 domains determine the subcellular localization and functional regulation of SHP2. SHP2 is widely expressed and involved in multiple cell signaling processes, such as Ras-Erk, PI3K-Akt, JakStat, Met, FGFR, EGFR, as well as insulin receptor and NF-kB pathways, and plays an important role in mitogenic, metabolic control, transcriptional regulation, cell migration and many other cellular functions.

SHP2 is associated with the development of various diseases, such as Noonan syndrome, as well as various forms of leukemia (e.g., juvenile myelomonocytic leukemia, acute myeloid leukemia) and various solid tumors (e.g., lung cancer, colon cancer, neuroblastoma, glioblastoma, melanoma, and liver cancer).

Around the development of SHP2 inhibitors, there are two major strategies: the development of inhibitors targeting the PTP catalytic region of SHP2 and the development of allosteric inhibitors targeting the non-catalytic region; due to the selectivity and poor druggability of PTP catalytic region inhibitors, currently more research tends towards the development of allosteric inhibitors. In recent years, researchers have found that inhibiting SHP2 activity through allosteric sites can improve activity and selectivity, and drug research has also made certain progress. However, there is still a need to develop better SHP2 inhibitors in order to obtain drugs with superior activity and better pharmacokinetic properties for the treatment of SHP2-mediated related diseases.

### SUMMARY

The purpose of the present invention is to provide a compound shown in formula I′ and its use in regulating SHP2 activity or treating SHP2 related diseases.

In the first aspect of the present invention, provided is a compound of formula I′, or a pharmaceutically acceptable salt, a stereoisomer, a solvate or a prodrug thereof,

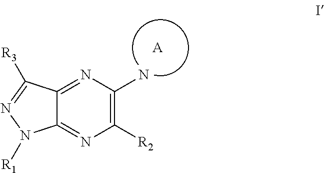

wherein, R₁ is selected from the group consisting of bicyclic C6-C10 aryl, 6-10 membered heteroaryl containing 1-3 heteroatoms selected from N, O, and S, C6-C10 aryl heterocycloalkyl; any hydrogen atom on R₁ is optionally substituted by one or more of the following substituents: deuterium, hydroxyl, halogen, cyano, ═O, ester, acylamino, ketocarbonyl, amino, hydroxyl-substituted C1-C4 alkyl, —C(O)ORₐ, —NHC(O)Rₐ, —NHC(O)ORₐ, —C(O)(C1-C4 alkylene)OH, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C10 aryl, and 6-10 membered heteroaryl containing 1-3 heteroatoms selected from N, O and S; Rₐ is C1-C4 alkyl; the C6-C10 aryl heterocycloalkyl is —(C6-C10 aryl) fused (saturated or unsaturated 3-8 membered heterocycloalkyl containing 1-3 heteroatoms selected from N, O and S); R₁ is a bicyclic and fused-ring structure;

R₂ is selected from the group consisting of H, deuterium, amino, cyano, halogen, hydroxyl, methyl, CH₂OH, CH(CH₃)OH, C(CH₃)₂OH, halomethyl, deuterated methyl, CONH₂, CF₂OH, NHSO₂Me, and CH₂NHSO₂Me;

R₃ is selected from the group consisting of hydrogen, deuterium, hydroxyl, amino, cyano, halogen, methyl, deuteromethyl, and halomethyl;

ring A is selected from the group consisting of: monocyclic or bicyclic 3-11 membered heterocycloalkyl containing 1-3 heteroatoms selected from N, O and S, 6-10 membered heteroaryl containing 1-3 heteroatoms selected from N, O and S, -(3-8 membered heterocycloalkylene containing 1-3 heteroatoms selected from N, O and S)-(3-8 membered heterocycloalkyl containing 1-3 heteroatoms selected from N, O and S), 4-8 membered bridged heterocycloalkyl containing 1-3 heteroatoms selected from N, O and S:

any hydrogen atom on ring A is unsubstituted or monosubstituted, disubstituted or trisubstituted by the following substituents: (CH₂),NHR′₁, (CH₂)ₙCONH₂, (CH₂)ₙCF₂H, (CH₂)ₙCF₃, (CH₂)ₙOH, ═O, C1-C6alkyl, halogen, amino, hydroxyl, —N—(C1-C6 alkyl), —(C1-C6 alkylene)—NH₂, wherein the hydrogen on the alkyl is unsubstituted or monosubstituted or disubstituted by OR′₁;

R′₁ is selected from the group consisting of: H, C1-C4 alkyl, and hydroxyl-substituted C1-C4 alkyl; and

n is selected from the group consisting of 0, 1, 2 and 3.

In another preferred embodiment, R₁ is selected from the group consisting of monocyclic or bicyclic C6-C10 aryl, 6-10 membered heteroaryl containing 1-3 heteroatoms selected from N, O, and S, C6-C10 aryl heterocycloalkyl; any hydrogen atom on Riis optionally substituted by one or more of the following substituents: deuterium, hydroxyl, halogen, cyano, ═O, ester, acylamino, ketocarbonyl, amino, hydroxyl-substituted C1-C4 alkyl, —C(O)ORₐ, —NHC(O)Rₐ, —NHC(O)ORₐ, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C10 aryl, and 6-10 membered heteroaryl containing 1-3 heteroatoms selected from N, O and S; Rₐ is C1-C4 alkyl;

R₂ is selected from the group consisting of H, deuterium, amino, cyano, halogen, hydroxyl, methyl, CH₂OH, CH(CH₃)OH, C(CH₃)₂OH, halomethyl, and deuterated methyl;

R₃ is selected from the group consisting of hydrogen, deuterium, hydroxyl, amino, cyano, halogen, methyl, deuteromethyl, and halomethyl;

ring A is selected from the group consisting of: monocyclic or bicyclic 3-11 membered heterocycloalkyl containing 1-3 heteroatoms selected from N, O and S, 6-10 membered heteroaryl containing 1-3 heteroatoms selected from N, O and S, -(3-8 membered heterocycloalkylene containing 1-3 heteroatoms selected from N, O and S)-(3-8 membered heterocycloalkyl containing 1-3 heteroatoms selected from N, O and S), and 4-8 membered bridged heterocycloalkyl containing 1-3 heteroatoms selected from N, O and S;

any hydrogen atom on ring A is unsubstituted or monosubstituted, disubstituted or trisubstituted by the following substituents: (CH₂)ₙNHR′₁, (CH₂)ₙCONH₂, (CH₂)ₙCF₂H, (CH₂)ₙCF₃, (CH₂)ₙOH, —O, C1-C6alkyl, halogen, amino, hydroxyl, —N—(C1-C6 alkyl), —(C1-C6 alkylene)—NH₂, wherein the hydrogen on the alkyl is unsubstituted or monosubstituted or disubstituted by OR′₁;

R′₁ is selected from the group consisting of: H, C1-C4 alkyl, and hydroxyl-substituted C1-C4 alkyl; and

n is selected from the group consisting of 0, 1, 2 and 3.

In another preferred embodiment, R₁ is a ring B-fused-ring C, wherein,

ring B and ring C are each independently selected from the group consisting of: C5-C6 aryl, 5-6 membered heteroaryl containing 1-3 heteroatoms selected from N, O and S, C5-C6 cycloalkyl, saturated 5-6 membered heterocycloalkyl containing 1-3 heteroatoms selected from N, O and S;

any hydrogen atom on R₁ is optionally substituted by one or more of the following substituents: deuterium, hydroxyl, halogen, cyano, ═O, amino, hydroxyl-substituted C1-C4 alkyl, C1-C6 alkyl, C1-C6 haloalkyl, C1-C6 thioalkyl, C1-C6 alkoxy, C3-C6 cycloalkyl, C1-C6 alkylamino, C6-C10 aryl, 6-10 membered heteroaryl containing 1-3 heteroatoms selected from N, O and S, and —C(O)C(CH₃)₂OH.

In another preferred embodiment, R₁ is selected from the group consisting of monocyclic or bicyclic C6-C10 aryl, and 6-10 membered heteroaryl containing 1-3 heteroatoms selected from N, O, and S;

any hydrogen atom on R₁ is optionally substituted by one or more of the following substituents: deuterium, hydroxyl, halogen, cyano, ═O, amino, hydroxyl-substituted C1-C4 alkyl, C1-C6 alkyl, C1-C6 haloalkyl, C1-C6 thioalkyl, C1-C6 alkoxy, C3-C6 cycloalkyl, C6-C10 aryl, and 6-10 membered heteroaryl containing 1-3 heteroatoms selected from N, O and S.

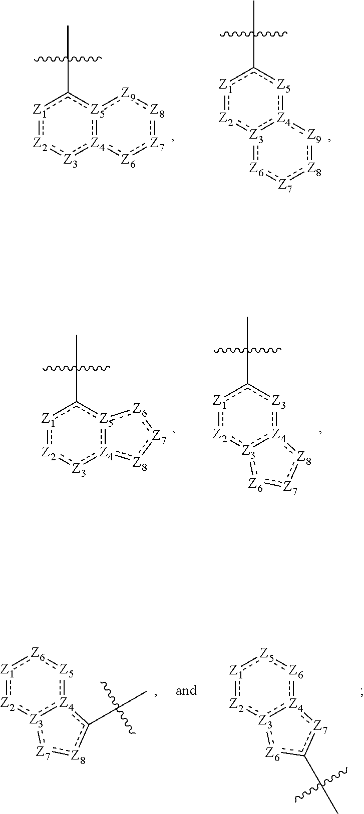

In another preferred embodiment, R₁ is selected from the group consisting of

Z₁, Z₂, Z₃, Z₄, Z₅, Z₆, Z₇, Z₈, and Z₉ are each independently selected from the group consisting of N, O, S, C, C(R₄)ₘ and NR₄;

each R₄ is independently selected from the group consisting of: hydrogen, deuterium, hydroxyl, halogen, cyano, ═O, amino, hydroxyl-substituted C1-C4 alkyl, C1-C6 alkyl, C1-C6 haloalkyl, C1-C6 thioalkyl, C1-C6 alkoxy, C3-C6 cycloalkyl, C1-C6 alkylamino, C6-C10 aryl, 6-10 membered heteroaryl containing 1-3 heteroatoms selected from N, O and S, and —COC(CH₃)₂OH;

[symbol] is a single or double bond;

each m is independently selected from the group consisting of 1 and 2.

In another preferred embodiment, R₁ is selected from the group consisting of

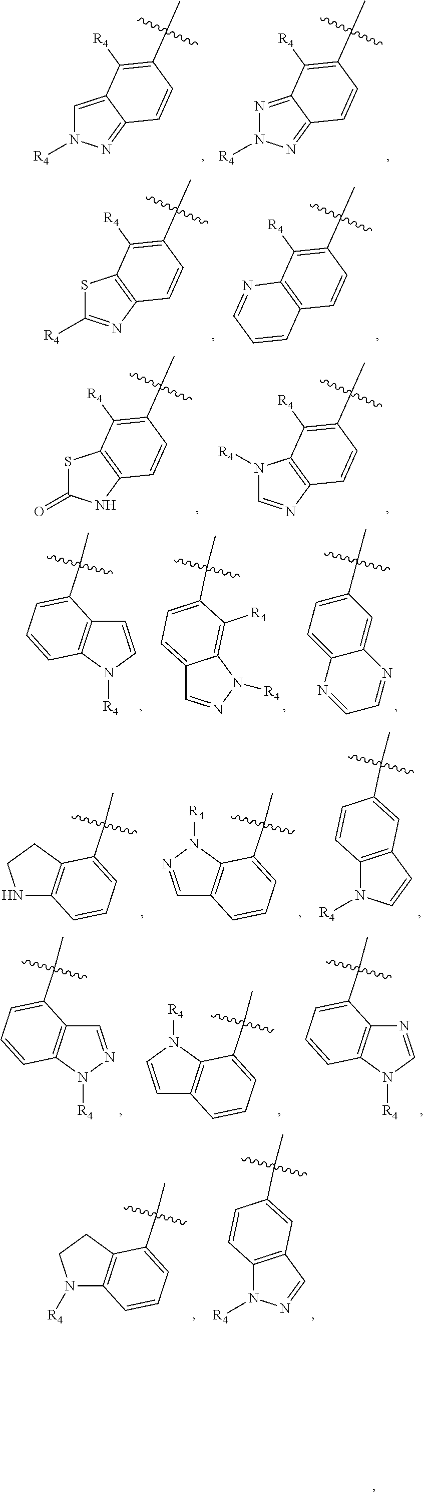

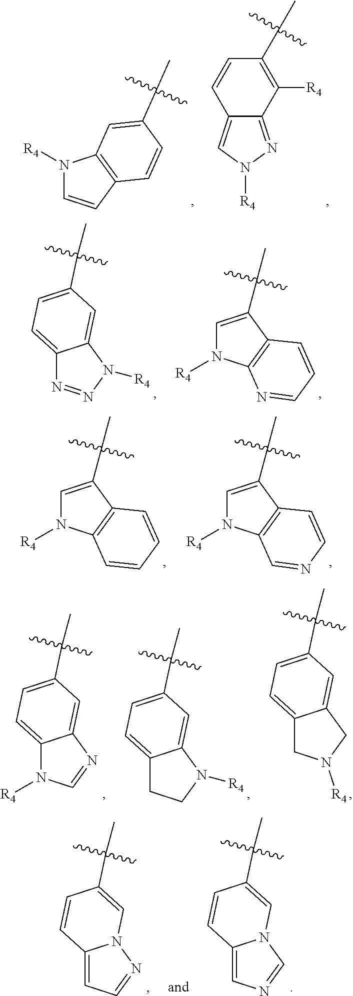

In another preferred embodiment, ring A is

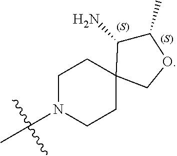

In another preferred embodiment, ring A is selected from the group consisting of: monocyclic or bicyclic 3-11 membered heterocycloalkyl containing 1-3 heteroatoms selected from N, O and S, 6-10 membered heteroaryl containing 1-3 heteroatoms selected from N, O and S, -(3-8 membered heterocycloalkylene containing 1-3 heteroatoms selected from N, O and S)-(3-8 membered heterocycloalkyl containing 1-3 heteroatoms selected from N, O and S), and 4-8 membered bridged heterocycloalkyl containing 1-3 heteroatoms selected from N, O and S;

any hydrogen atom on ring A is unsubstituted or monosubstituted, disubstituted or trisubstituted by the following substituents: (CH₂)NHR′₁, (CH₂)ₙCONH₂, (CH₂)ₙCF₂H, (CH₂)ₙCF₃, (CH₂)ₙOH, ═O, C1-C6alkyl, halogen, amino, hydroxyl, —N—(C1-C6 alkyl), —(C1-C6 alkylene)—NH₂, wherein the hydrogen on the alkyl is unsubstituted or monosubstituted or disubstituted by OR′₁;

R′₁ is selected from the group consisting of: H, C1-C4 alkyl, and hydroxyl-substituted C1-C4 alkyl; and

n is selected from the group consisting of 0, 1, 2 and 3.

In another preferred embodiment, the bicyclic 3-11 membered heterocycloalkyl containing 1-3 heteroatoms selected from N, O, and S has a spiro ring structure.

In another preferred embodiment, the compound is selected from the group consisting of:

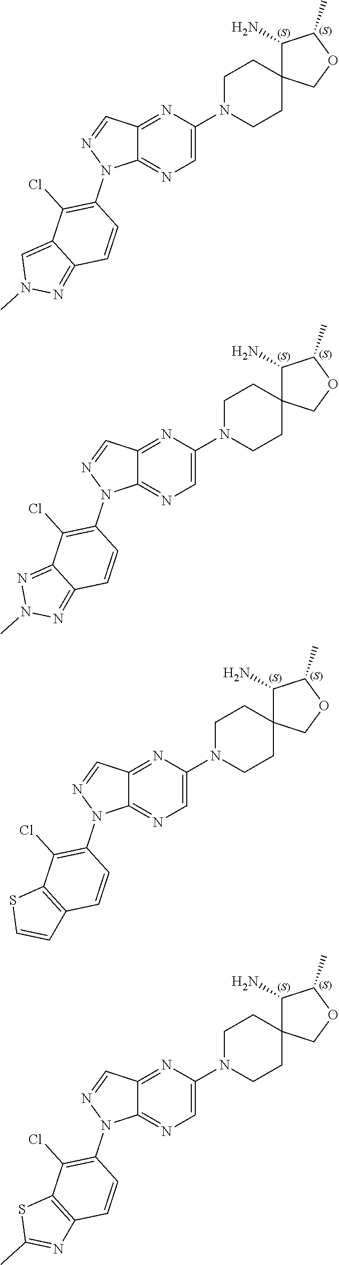

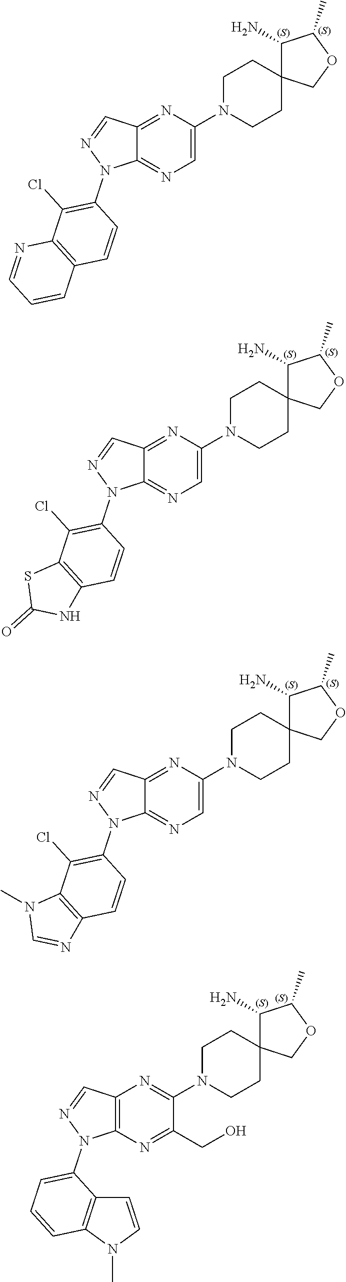

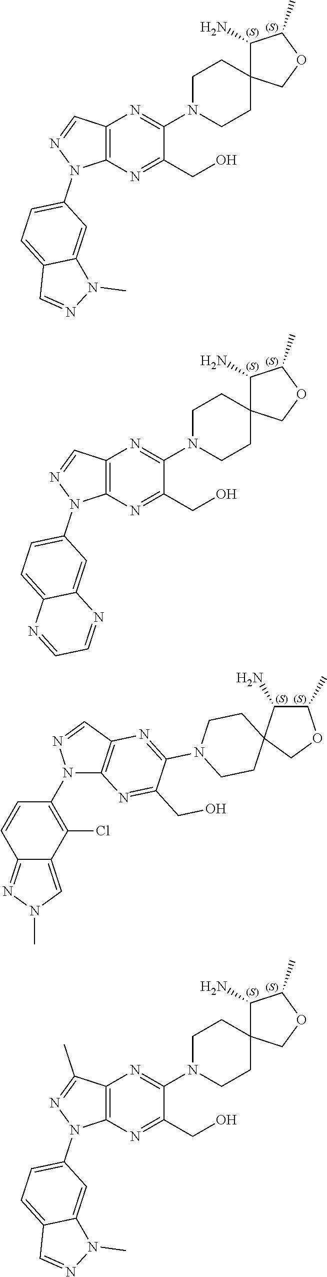

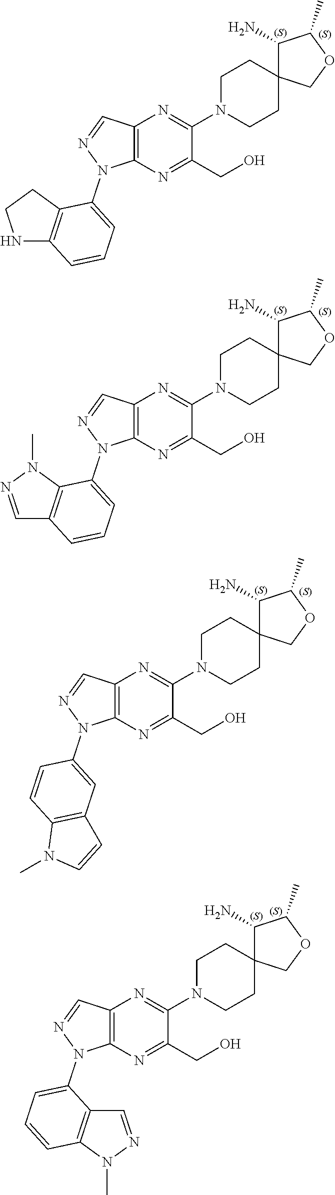

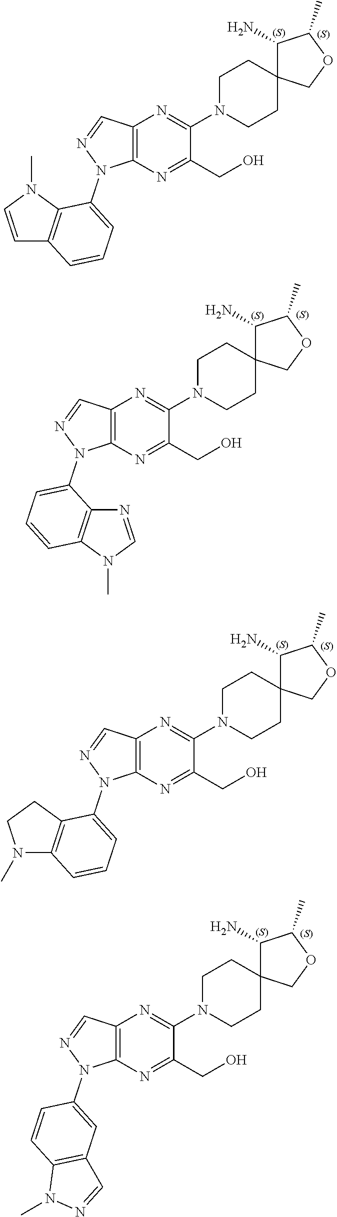

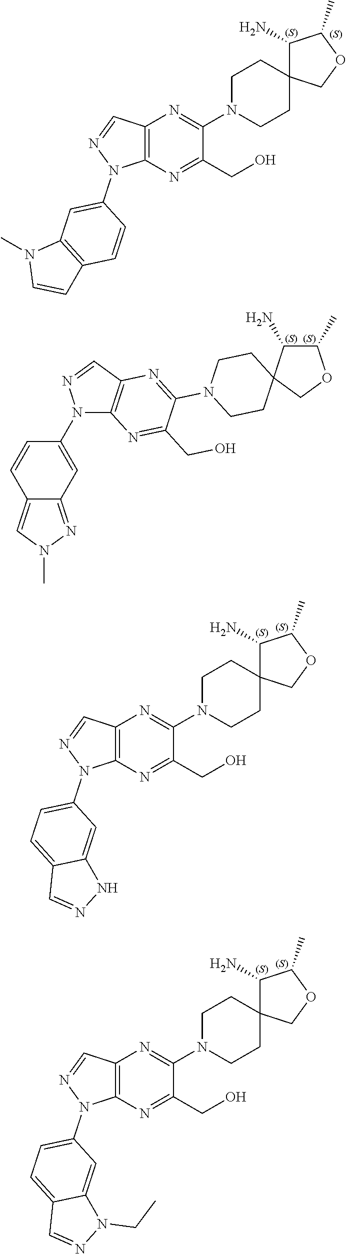

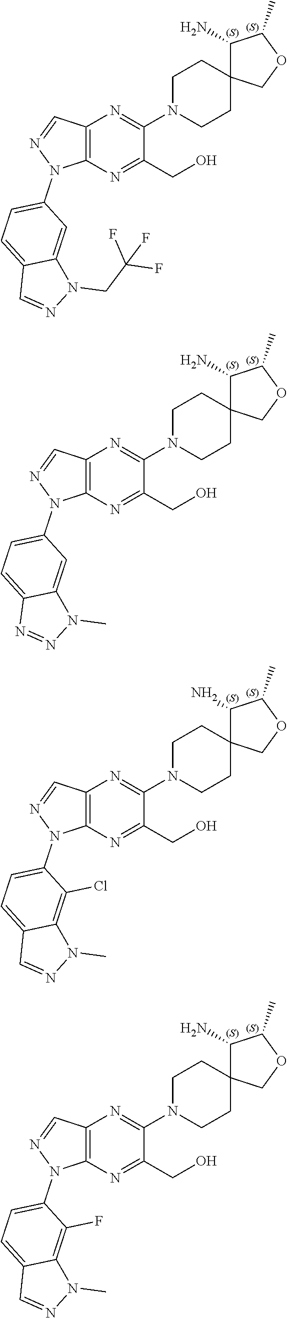

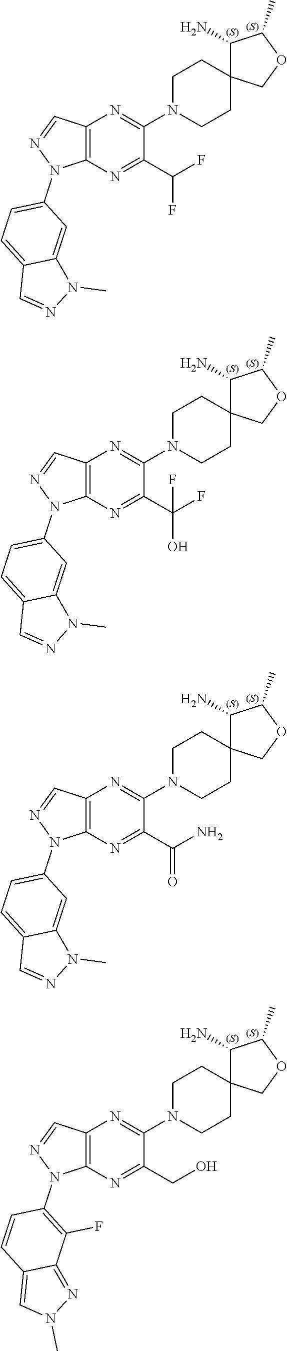

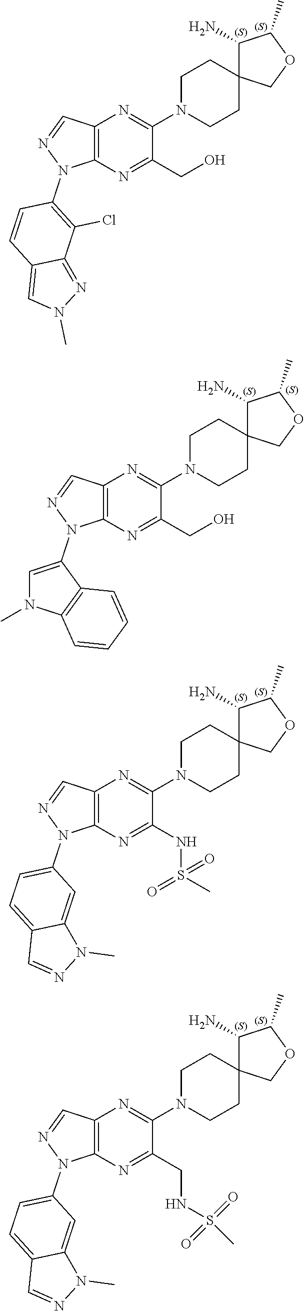

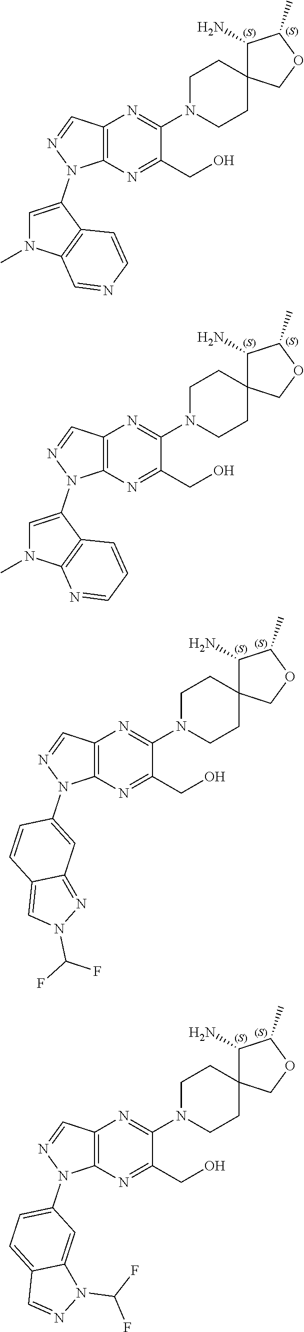

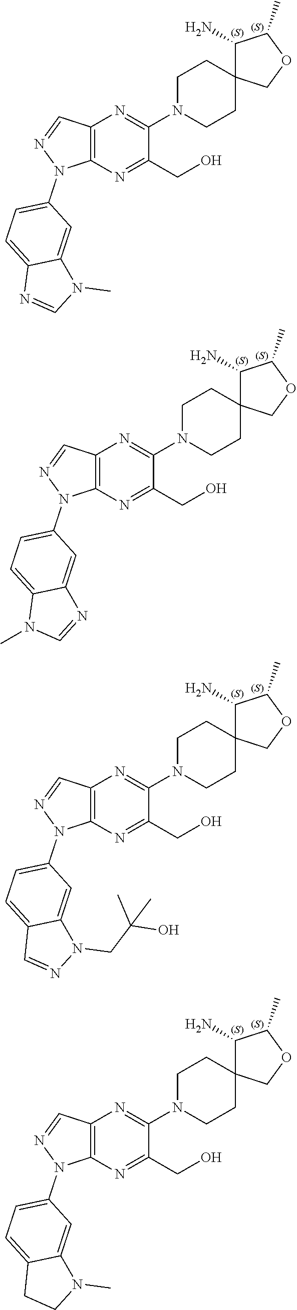

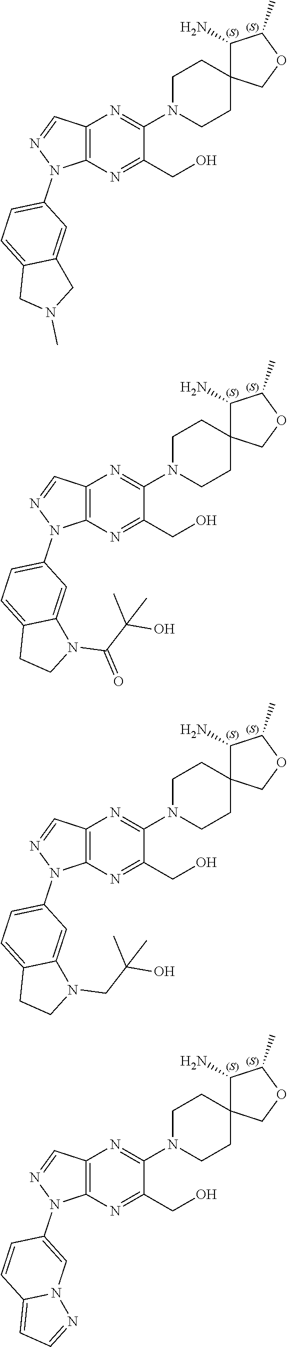

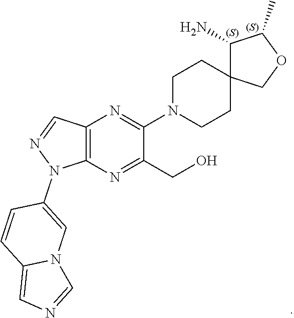

In another preferred embodiment, the compound is selected from the group consisting of:

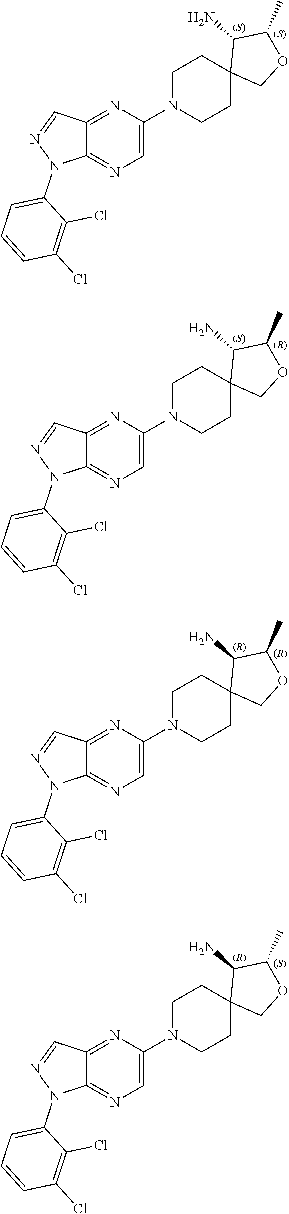

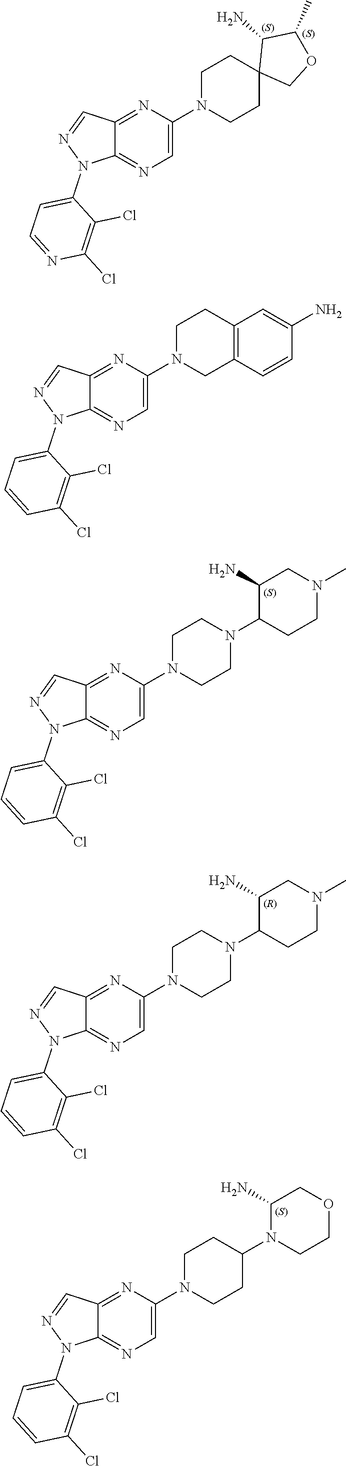

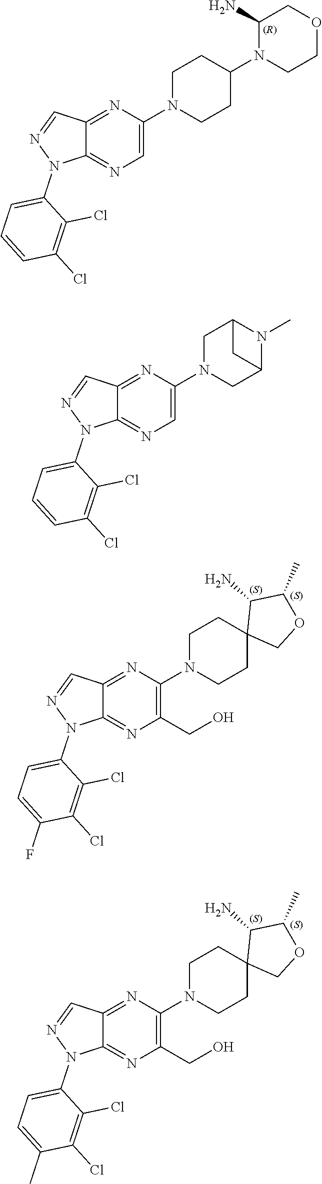

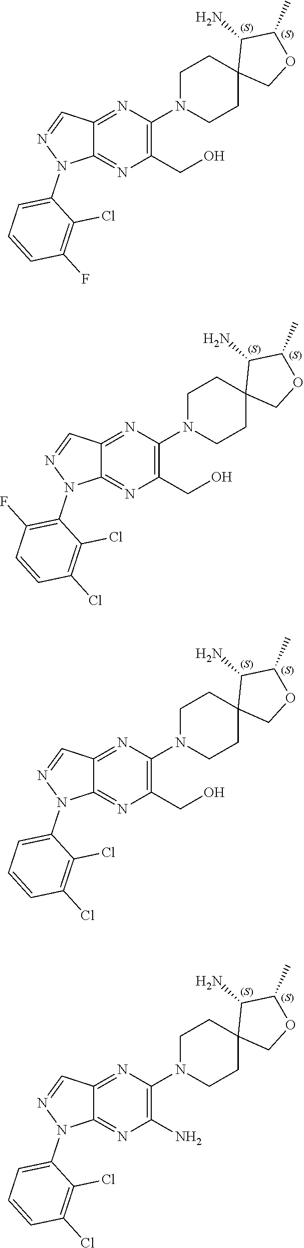

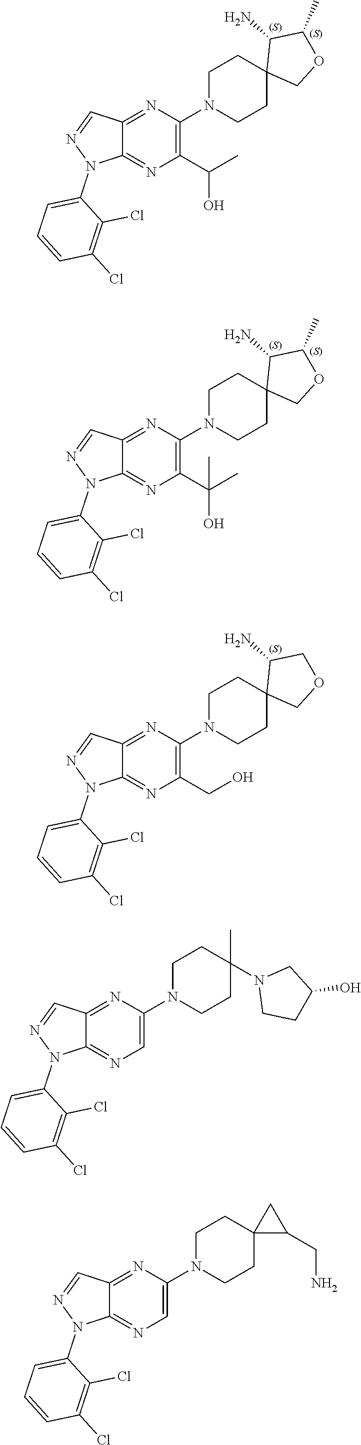

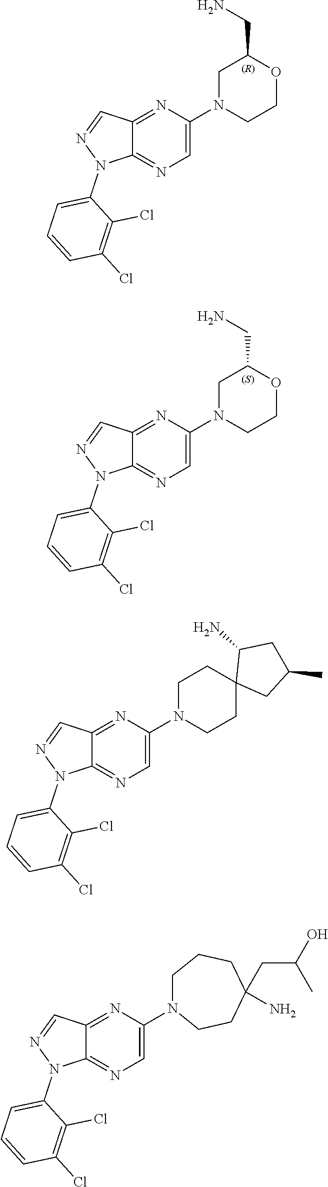

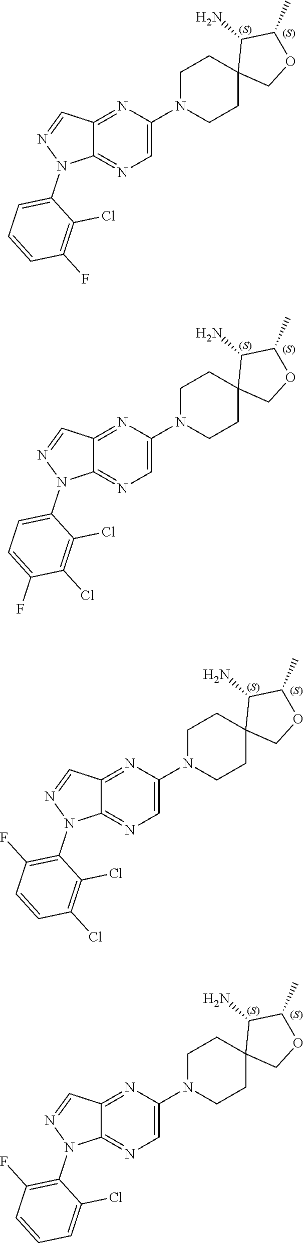

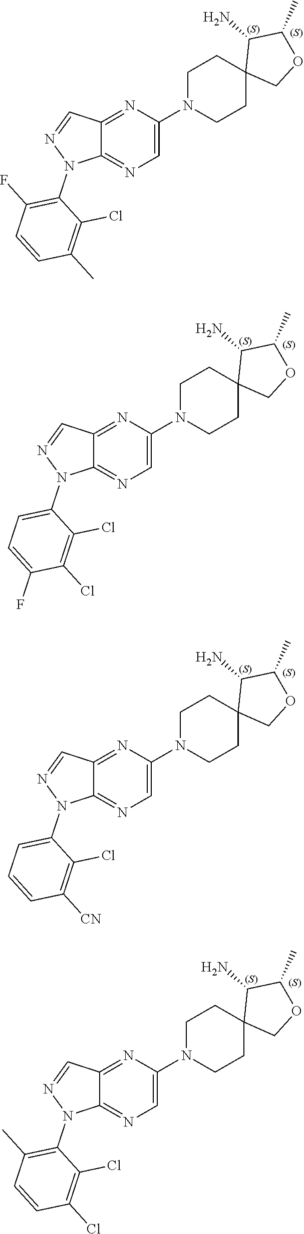

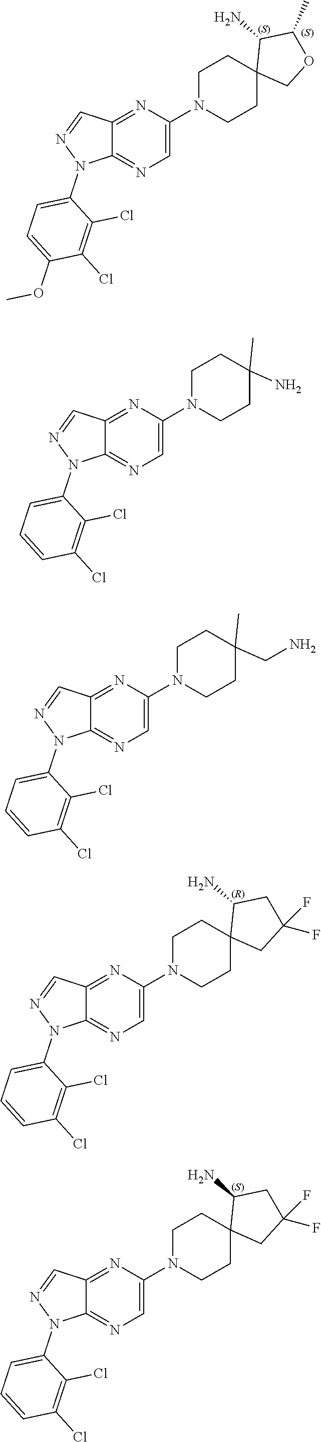

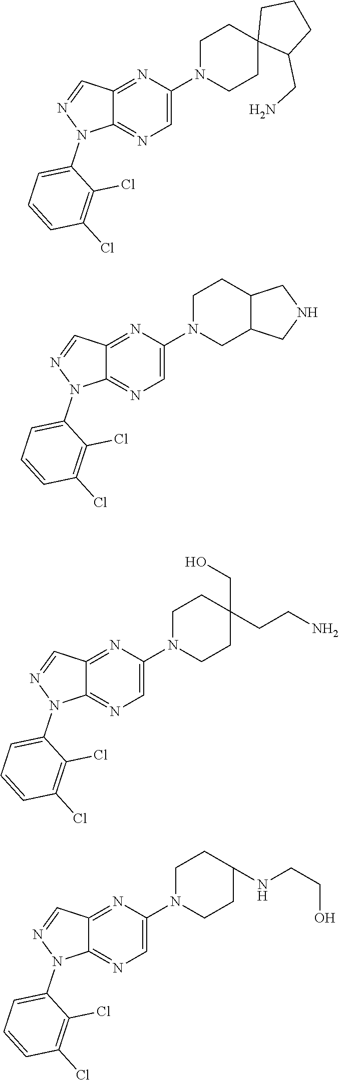 [SMILES: NCC1CCCC12CCN(c1cnc3c(cnn3-c3cccc(Cl)c3Cl)n1)CC2 ; Clc1cccc(-n2ncc3nc(N4CCC5CNCC5C4)cnc32)c1Cl ; NCCC1(CO)CCN(c2cnc3c(cnn3-c3cccc(Cl)c3Cl)n2)CC1 ; OCCNC1CCN(c2cnc3c(cnn3-c3cccc(Cl)c3Cl)n2)CC1]

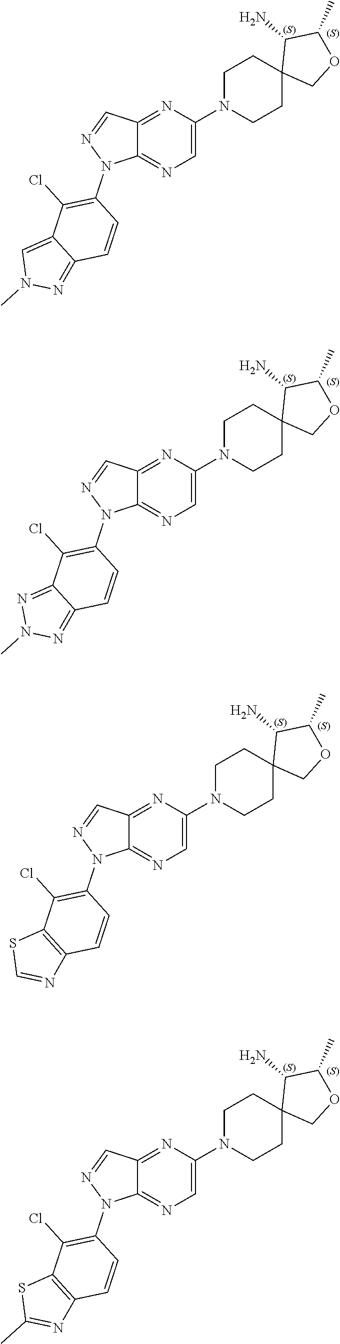

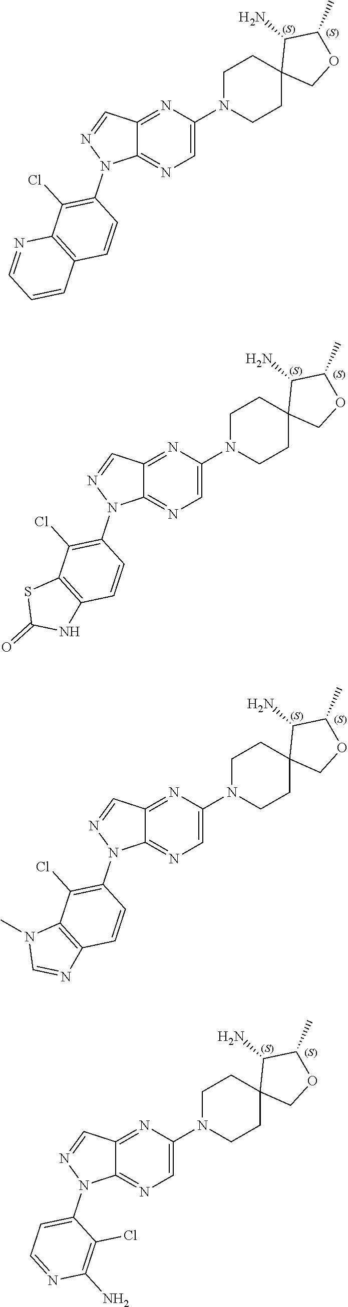

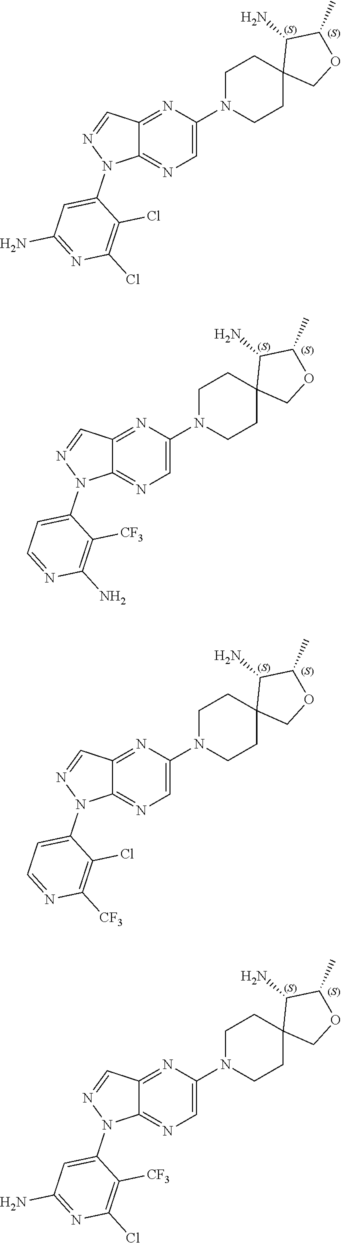

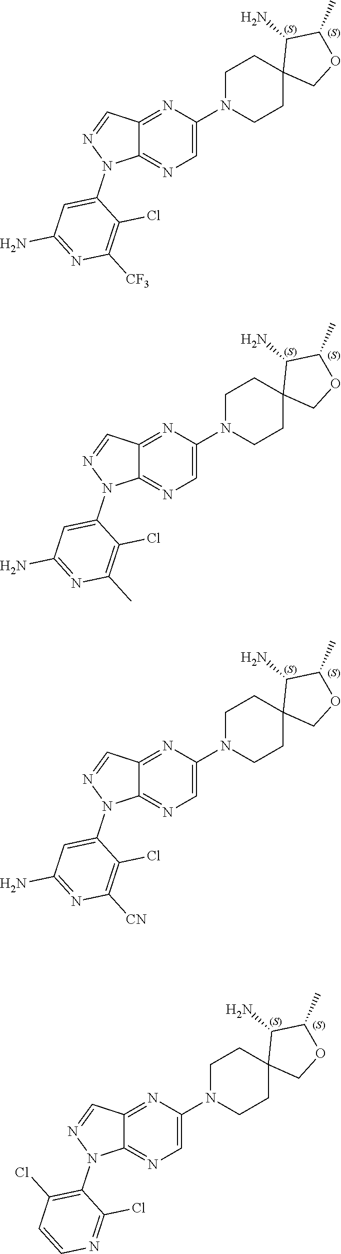

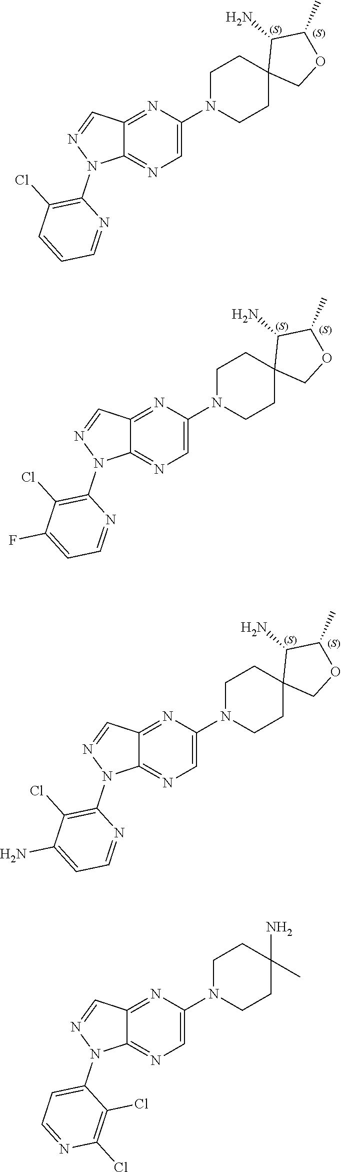

In another preferred embodiment, the pharmaceutically acceptable salt is an inorganic acid salt or an organic acid salt.

In another preferred embodiment, the inorganic acid salt is selected from the group consisting of hydrochloride, hydrobromide, hydroiodide, sulfate, bisulfate, nitrate, phosphate, and acid phosphate.

In another preferred embodiment, the organic acid salt is selected from the group consisting of formate, acetate, trifluoroacetate, propionate, pyruvate, glycolate, oxalate, malonate, fumarate, maleate, lactate, malate, citrate, tartrate, methanesulfonate, ethanesulfonate, benzenesulfonate, salicylate, picrate, glutamate, ascorbate, camphorate, and camphorsulfonate.

In the second aspect of the present invention, provided is a pharmaceutical composition, comprising a pharmaceutically acceptable carrier and one or more safe and effective amounts of the compound as described in the first aspect of the present invention, or a pharmaceutically acceptable salt, a stereoisomer, a solvate or a prodrug thereof.

In the third aspect of the present invention, provided is a use of the pharmaceutical composition described in the second aspect of the present invention in the preparation of a medicament used as a SHP2 inhibitor.

In the fourth aspect of the present invention, provided is a use of the pharmaceutical composition as described in the second aspect of the present invention in the preparation of a medicament for regulating SHP2 activity or treating SHP2-related diseases.

In another preferred embodiment, the SHP2-related disease is selected from the group consisting of Noonan syndrome, Leopard syndrome, juvenile myelomonocytic leukemia, acute myeloid leukemia, neuroblastoma, melanoma, breast cancer, esophageal cancer, lung cancer, gastric cancer, head cancer, anaplastic large cell lymphoma, neuroblastoma, glioblastoma, squamous cell carcinoma of the head and neck, colon cancer, and liver cancer.

It should be understood that within the disclosure of the present invention, the above-mentioned technical features of the present invention and the technical features specifically described in the following (such as examples) can be combined with each other to form new or preferred technical solutions. Due to space limitations, it will not be repeated here.

### DETAILED DESCRIPTION OF THE INVENTION

After long-term and in-depth research, the inventors have obtained a class of compounds with higher inhibitory effect on the activity of SHP2 phosphatase through structural design. On this basis, the inventors have completed the present invention.

### Terms

In the present invention, unless otherwise specified, the terms used herein have the ordinary meaning known to those skilled in the art.

In the present invention, the term “halogen” refers to F, Cl, Br or I.

In the present invention, “C1-C6 alkyl” refers to a linear or branched alkyl having 1-6 carbon atoms, such as methyl, ethyl, propyl, isopropyl, butyl, isobutyl, tert-butyl, neopentyl, tert-amyl, or similar groups. “C1-C4 alkyl” has a similar meaning.

In the present invention, the term “C2-C6 alkenyl” refers to a linear or branched alkenyl having 2-6 carbon atoms and a double bond, including without limitation ethenyl, propenyl, butenyl, isobutenyl, pentenyl and hexenyl etc.

In the present invention, the term “C2-C6 alkynyl” refers to a linear or branched alkynyl having 2-6 carbon atoms and a triple bond, including without limitation ethynyl, propynyl, butynyl, isobutynyl, pentynyl and hexynyl, etc.

In the present invention, the term “C3-C8 cycloalkyl” refers to a cyclic alkyl having 3-8 carbon atoms on the ring, including without limitation cyclopropyl, cyclobutyl, cyclopentyl, cyclohexyl, cycloheptyl, cyclooctyl, etc. “C3-C6 cycloalkyl” and “C5-C6 cycloalkyl” have similar meanings.

In the present invention, the term “C1-C6 alkoxy” refers to a linear or branched alkoxy having 1-6 carbon atoms, including without limitation methoxy, ethoxy, propoxy, isopropoxy and butoxy, etc. Preferably, C1-C4 alkoxy.

In the present invention, the term “heterocycloalkyl” is a 3-11 membered heterocyclic containing 1, 2 or 3 heteroatoms selected from N, O and S, including (but not limited to) the following groups:

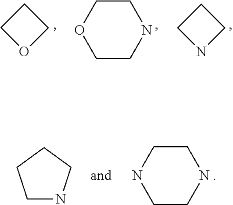

In the present invention, the term “aromatic ring” or “aryl” has the same meaning, preferably “C6-C10 aryl”. The term “C6-C10 aryl” refers to an aromatic ring group having 6 -10 carbon atoms without heteroatoms on the ring, such as phenyl, naphthyl and the like.

In the present invention, the term “heteroaromatic ring” or “heteroaryl” has the same meaning and refers to a heteroaromatic group containing one to more heteroatoms. For example, “C3-C10 heteroaryl” refers to a heteroaromatic ring containing 1-4 heteroatoms selected from oxygen, sulfur and nitrogen and 3-10 carbon atoms. Non-limiting examples include: furyl, thienyl, pyridyl, pyrazolyl, pyrrolyl, N-alkylpyrrolyl, pyrimidinyl, pyrazinyl, imidazolyl, tetrazolyl, and the like. The heteroaryl ring may be fused to an aryl, heterocyclyl or cycloalkyl ring, wherein the ring bonded to the parent structure is a heteroaryl ring. Heteroaryl can be optionally substituted or unsubstituted.

In the present invention, the term “halo” refers to being substituted by halogen.

In the present invention, the term “deuterated” refers to being substituted by deuterium.

In the present invention, the term “substituted” refers to that one or more hydrogen atoms on a specific group are substituted by a specific substituent. The specific substituents are the corresponding substituents described above, or the substituents appearing in each example. Unless otherwise specified, a substituted group may have a substituent selected from a specific group at any substitutable position of the group, and the substituents may be the same or different at each position. Those skilled in the art will appreciate that combinations of substituents contemplated by this invention are those that are stable or chemically feasible. The substituents are for example (but not limited to): halogen, hydroxyl, carboxyl (—COOH), C1-C6 alkyl, C2-C6 alkenyl, C2-C6 alkynyl, C3-C8 cycloalkyl, 3- to 12-membered heterocyclyl, aryl, heteroaryl, C1-C8 aldehyde, C2-C10 acyl, C2-C10 ester group, amino, C1-C6 alkoxy, C1-C10 sulfonyl, etc.

In the present invention, the term “more” refers to 1-7.

In the present invention, the term 1-6 refers to 1, 2, 3, 4, 5 or 6. Other similar terms have similar meanings.

The term “ester group” has the structure of —C(O)—O—R′ or R′—C(O)—O—, wherein R′ independently represents hydrogen, C1-C6 alkyl, C3-C6 cycloalkyl , C6-C10 aryl, heteroaryl, heterocyclyl, as defined above.

The term “ketocarbonyl” has R—C(═O)—, wherein R is alkyl, cycloalkyl as described above, etc.

The term “acylamino” refers to a group with the structure —CONRR′, wherein R and R′ can independently represent hydrogen, alkyl or substituted alkyl, cycloalkyl or substituted cycloalkyl, aryl or substituted aryl, heterocycle or substituted heterocycle, as defined above. R and R′ can be the same or different in the dialkylamine moiety.

The term “C6-C10 arylheterocycloalkyl” refers to a fused ring structure containing 6 to 10 carbon atoms formed by a heteroatom-containing cycloalkyl and an aryl, or a heteroatom-containing cycloalkyl is attached to the aryl group via an atom thereon.

The term “amino” refers to —NH₂.

The term “C1-C6 heteroalkyl” refers to a substituted alkyl having one or more framework chain atoms selected from atoms other than carbon, for example, oxygen, nitrogen, sulfur, phosphorus, Si, or combinations thereof. Numerical ranges may be given, e.g., C1-C6 heteroalkyl refers to the number of carbons in the chain, which includes 1 to 6 carbon atoms. For example —CH₂OCH₂CH₃ is referred to as a “C3” heteroalkyl. It can be attached to the rest of the molecule via heteroatoms or carbon in the heteroalkyl chain. Examples of “heteroalkyl” include, but are not limited to: CH₂OCH₃, CH₂CH₂OCH₃, CH₂NHCH₃, CH₂CH₂NHCH₃, Me₃Si,Me₃SiCH₂CH₂O, Me₃SiCH₂CH₂OCH₂-(SEM). “Heteroalkylene” refers to an optionally substituted divalent alkyl having one or more framework chain atoms selected from atoms other than carbon, for example, oxygen, nitrogen, sulfur, phosphorus, Si, or combinations thereof.

The term “C1-C6 alkylamino” refers to a group having an alkyl-NR— structure, wherein R is H, or the above-mentioned alkyl, cycloalkyl, aryl, heteroaryl, etc.

The term “C3-C8 cycloalkylamino” refers to a group of the formula —NRaRb, wherein Ra is H, an alkyl as defined herein or a cycloalkyl as defined herein, and Rb is a cycloalkyl as defined herein, or Ra and Rb together with the N atom they are attached to form a 3-10 membered N-containing monocyclic or bicyclic heterocyclic group, such as tetrahydropyrrolyl. As used herein, C3-C8 cycloalkylamino refers to amino containing 3-8 carbon atoms.

The term “bridged heterocycloalkyl” refers to a heterocycloalkyl having bridging carbon atoms.

### Compound

The present invention provides a compound of formula I′, or a pharmaceutically acceptable salt, a stereoisomer, a solvate or a prodrug thereof,

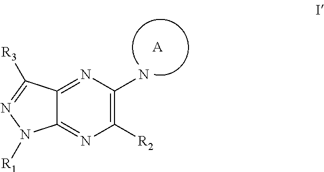

wherein, each group is defined as above.

In another preferred embodiment, in the compound, any one of R₁, R₂, R₃ and ring A is independently the corresponding group in the specific compound of the present invention.

As used herein, the term “pharmaceutically acceptable salt” refers to a salt of a compound of the present invention formed with an acid or a base which is suitable for use as a medicine. Pharmaceutically acceptable salts include inorganic salts and organic salts. A preferred class of salts is the salts of the compounds of the invention with acids. Acids suitable for forming salts include, but are not limited to: inorganic acids such as hydrochloric acid, hydrobromic acid, hydrofluoric acid, sulfuric acid, nitric acid, phosphoric acid, etc.; organic acids such as formic acid, acetic acid, trifluoroacetic acid, propionic acid, oxalic acid, malonic acid, succinic acid, fumaric acid, maleic acid, lactic acid, malic acid, tartaric acid, citric acid, picric acid, benzoic acid, methanesulfonic acid, ethanesulfonic acid, p-toluenesulfonic acid, benzenesulfonic acid, naphthalenesulfonic acid, etc.; and amino acids such as proline, phenylalanine, aspartic acid, glutamic acid, etc.

Another preferred class of salts are the salts of the compounds of the present invention with bases, such as alkali metal salts (e.g. sodium or potassium salts), alkaline earth metal salts (e.g. magnesium or calcium salts), ammonium salts (e.g. lower alkanol ammonium salts and other pharmaceutically acceptable amine salts), such as methylamine salts, ethylamine salts, propylamine salts, dimethylamine salts, trimethylamine salts, diethylamine salts, triethylamine salts, tert-butyl amine salts, ethylenediamine salts, hydroxyethylamine salts, dihydroxyethylamine salts, trihydroxyethylamine salts, and amine salts formed with morpholine, piperazine, and lysine, respectively.

The term “solvate” refers to a complex in which a compound of the present invention coordinates with solvent molecules in a specific ratio.

The term “prodrug” includes itself, and may be biologically active or inactive, and when taken in an appropriate manner, it undergoes metabolism or chemical reactions in human body to convert into a class of compounds of formula I′, or a salt or solution of a compound of formula I′. The prodrugs include (but are not limited to) carboxylates, carbonates, phosphates, nitrates, sulfates, sulfone esters, sulfoxide esters, amino compounds, carbamates, azo compounds, phosphoramide, glucoside, ether, acetal and other forms of the compound.

### Pharmaceutical Composition and Mode of Administration

The present invention also provides a pharmaceutical composition comprising a pharmaceutically acceptable carrier and one or more safe and effective amounts of the compound of the present invention, or pharmaceutically acceptable salts, stereoisomers, solvates, or prodrugs thereof.

The pharmaceutical composition of the present invention includes compounds of the present invention or pharmaceutically acceptable salts thereof within a safe and effective range, as well as pharmaceutically acceptable excipients or carriers. The “safe and effective amount” refers to the amount of compound that is sufficient to significantly improve the condition without causing serious side effects. Typically, the pharmaceutical composition contains 1-2000 mg compound of the present invention/formulation, and more preferably, 10-1000 mg compound of the present invention/formulation. Preferably, “one dose” is a capsule or tablet.

“Pharmaceutically acceptable carrier” refers to one or more compatible solid or liquid fillers or gel substances, which are suitable for human use and must have sufficient purity and low toxicity. “Compatibility” herein refers to the ability of components of a composition to blend with each other and with the compounds of the invention without significantly reducing the efficacy of the compounds. Examples of pharmaceutically acceptable carriers include cellulose and its derivatives (such as sodium carboxymethyl cellulose, sodium ethyl cellulose, cellulose acetate, etc.), gelatin, talc, solid lubricants (such as stearic acid, magnesium stearate), calcium sulfate, vegetable oil (such as soybean oil, sesame oil, peanut oil, olive oil, etc.), polyols (such as propylene glycol, glycerin, mannitol, sorbitol, etc.), emulsifiers (such as Tween®), wetting agents (such as sodium dodecyl sulfate), colorants, flavoring agents, stabilizers, antioxidants, preservatives, non-thermal raw water, etc.

The pharmaceutical composition is an injection, a capsule, a tablet, a pill, powder or granule.

The mode of administration of the compounds or pharmaceutical compositions of the present invention is not particularly limited, and representative mode of administration include, but are not limited to, oral, intratumoral, rectal, parenteral (intravenous, intramuscular or subcutaneous), and topical administration.

Solid dosage forms for oral administration include capsules, tablets, pills, powders and granules. In these solid dosage forms, the active compound is mixed with at least one conventional inert excipient (or carrier), such as sodium citrate or dicalcium phosphate, or with the following ingredients: (a) filler or compatibilizer, such as starch, lactose, sucrose, glucose, Mannitol and silicic acid; (b) adhesives, such as hydroxymethyl cellulose, alginate, gelatin, polyvinylpyrrolidone, sucrose and arabicgum; (c) humectant, such as glycerol; (d) disintegrating agents, such as agar, calcium carbonate, potato starch or cassava starch, algic acid, some complex silicates, and sodium carbonate; (e) slow solvent, such as paraffin; (f) absorption accelerators, such as quaternary amine compounds; (g) wetting agents, such as cetyl alcohol and glycerol monostearate; (h) adsorbents, such as kaolin; and (i) lubricants, such as talc, calcium stearate, magnesium stearate, solid polyethylene glycol, sodium dodecyl sulfate, or mixtures thereof. In capsules, tablets and pills, dosage forms may also contain buffers.

Solid dosage forms such as tablets, sugar pills, capsules, pills and granules may be prepared using coating and shell materials such as casing and other materials well known in the art. They may comprise an opacifying agent, and the release of the active compound in such a composition may be released in a delayed manner in a part of the digestive tract. Examples of embedding components that can be employed are polymeric substances and wax substances. If necessary, the active compound may also form a microcapsule form with one or more of the excipients described above.

Liquid dosage forms for oral administration include pharmaceutically acceptable emulsions, solutions, suspensions, syrups or tinctures. In addition to the active compounds, the liquid dosage form may contain inert diluents conventionally used in the art, such as water or other solvents, solubilizers and emulsifiers, for example, ethanol, isopropanol, ethyl carbonate, ethyl acetate, propylene glycol, 1,3-butanediol, dimethylformamide and oils, especially cottonseed oil, peanut oil, corn germ oil, olive oil, castor oil and sesame oil, or mixtures thereof.

In addition to these inert diluents, the composition may also contain auxiliaries such as wetting agents, emulsifiers, suspending agents, sweeteners, flavoring agents and flavors.

In addition to the active compound, the suspension may comprise suspending agents, such as ethoxylated isooctadecanol, polyoxyethylene sorbitol and dehydrated sorbitol esters, microcrystalline cellulose, methanolic aluminum, agar, and any mixtures thereof.

The composition for parenteral injection may comprise physiologically acceptable sterile aqueous or anhydrous solutions, dispersions, suspensions or emulsions, and sterile powders for redissolution into sterile injectable solutions or dispersions. Suitable aqueous and non-aqueous carriers, diluents, solvents, or excipients include water, ethanol, polyols, and suitable mixtures thereof.

Dosage forms of the compound of the invention for topical administration include ointments, powder, patches, propellants and inhalants. The active ingredient is mixed under sterile conditions with a physiologically acceptable carrier and any preservatives, buffers or propellants as may be required.

The compound of the present invention can be administered alone or in combination with other pharmaceutically acceptable compounds (such as anti-tumor drugs).

The treatment method of the present invention can be administered alone or in combination with other treatment methods or drugs.

When using the pharmaceutical composition, a safe and effective amount of the compound of the present invention is administered to mammals in need of treatment (such as humans), where the dosage at the time of administration is a dose considered pharmaceutically effective, which is typically 1 to 2,000 mg per day, more preferably 50 to 1000 mg per day for a 60 kg body weight human. Of course, the specific dosage should also take the route of administration, the patient's health and other factors into consideration, which are within the skill range of skilled doctors.

Compared with prior art, the main advantages of the present invention include:
(1) The compounds of the present invention have excellent SHP2 inhibitory activity;
(2) The compounds of the present invention have good bioavailability as well as lower toxicity.

The present invention is further described below in conjunction with specific embodiments. It is to be understood that these examples are intended to illustrate the invention only and not to limit the scope of the invention. The experimental methods that do not indicate specific conditions in the following examples usually follow the conventional conditions, such as those described in Sambrook et al., Molecular Cloning: A Laboratory Manual (New York: Cold Spring Harbor Laboratory Press, 1989), or the conditions suggested by the manufacturer. Unless otherwise specified, percentages and portions are calculated by weight.

Unless otherwise defined, all professional and scientific terms used herein have the same meanings as commonly understood by those skilled in the art. In addition, any methods and materials similar or equivalent to those described can be applied to the method of the present invention. The preferred implementation methods and materials described herein are for demonstration purposes only.

### Synthesis of Intermediate A

The synthetic route is as follows:

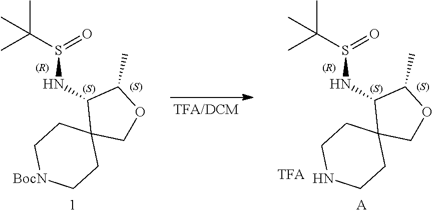

Compound 1 (2 g, 5.3 mmol) was dissolved in a solvent mixture of trifluoroacetic acid and dichloromethane (7 mL/21 mL) under the cooling of an ice-water bath, and then the reaction solution was moved to room temperature and stirred for 40 min. After completion of the reaction, the reaction was concentrated under reduced pressure to give 2.5 g of compound A trifluoroacetate. Ms [M+H]+ 275.3

NMR data of Compound A: 1H NMR (400 MHz, Chloroform-d) δ4.27-4.22(m, 1H), 4.04(d, J=10.8 Hz, 1H), 3.92(d, J=9.6 Hz, 1H), 3.66-3.57(m, 2H), 3.54-3.42(m, 2H), 3.16-3.05(m, 2H), 2.25-2.08(m, 2H), 1.87-1.73(m, 2H), 1.31(s, 9H), 1.24(d, J=6.4 Hz, 3H).

### Synthesis of Intermediate B

The synthetic route is as follows:

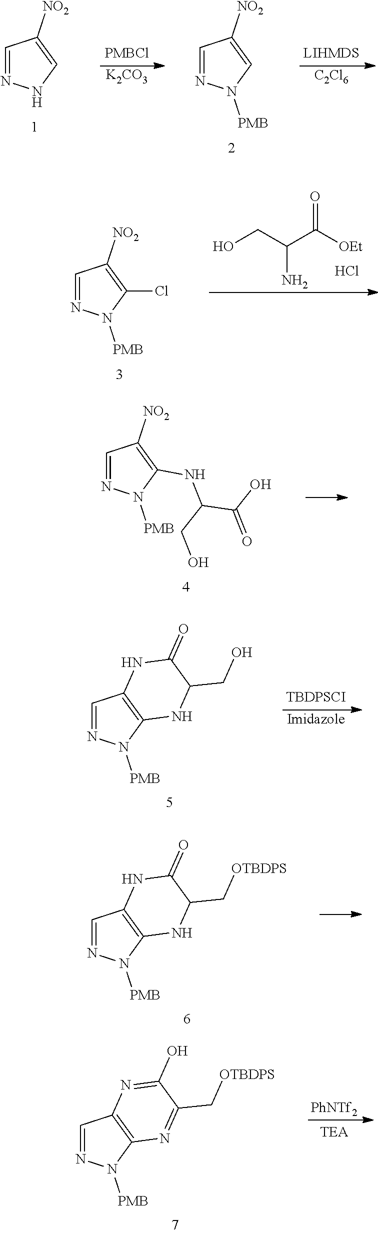

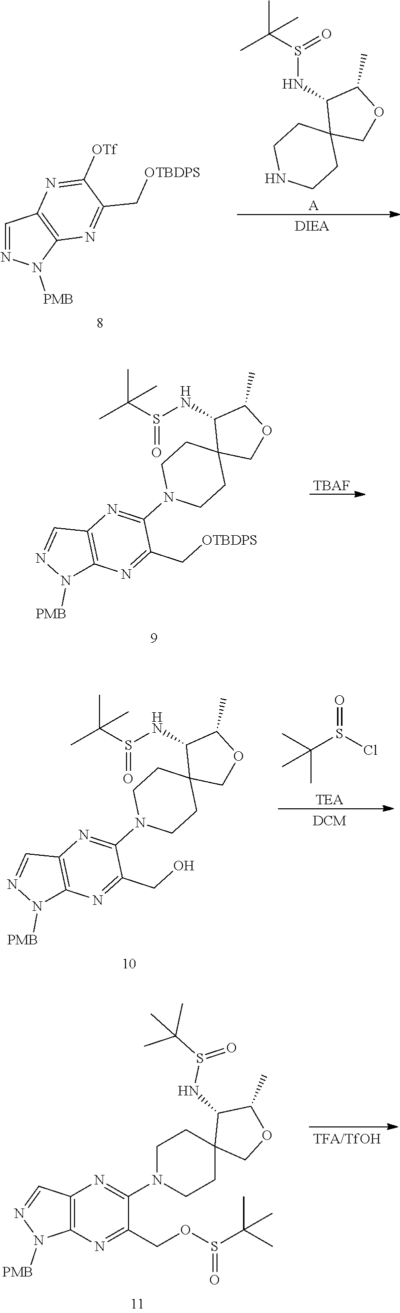

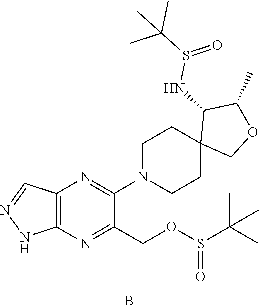

### 1. Synthesis of Compound 2

Compound 1 (54.7 g, 0.48 mol) was dissolved in 440 mL of N,N-dimethylformamide, then 4-methoxybenzyl chloride (83.6 g, 0.53 mol) and potassium carbonate (100.2 g, 0.73 mol) were added sequentially, and the reaction was heated up to 80° C. for 3 hours. After the completion of the reaction, the reaction solution was cooled to room temperature and added dropwise into 2 L of water, then extracted with ethyl acetate; the organic phases were combined, washed with saturated sodium chloride solution, dried with anhydrous sodium sulfate, filtered, and the filtrate was concentrated under reduced pressure and purified to give 110 g of compound 2.

NMR data of Compound 2: 1H NMR (400 MHz, Chloroform-d) δ8.01(s, 1H), 7.92(s, 1H), 7.19-7.17(d, J=8.39 Hz, 2H), 6.87-6.85(d, J=8.39 Hz, 2H), 5.17(s, 2H), 3.75(s, 3H).

### 2. Synthesis of Compound 3

Compound 2 (50.6 g, 0.22 mol) was dissolved in 500 mL of anhydrous tetrahydrofuran, under nitrogen protection, the reaction solution was cooled to −60° C., and added dropwise with 260 mL solution of 1 M Lithium bis(trimethylsilyl)amide in tetrahydrofuran. The reaction solution was stirred at −60° C. for 1 hr after completion of the addition and then a solution of hexachloroethane (61.6 g, 0.26 mol) in 300 mL of anhydrous tetrahydrofuran was added dropwise at −60° C. and stirred for 1 hour. After completion of the reaction, 1 L of saturated ammonium chloride solution was added dropwise at −60° C. to quench the reaction, then the reaction solution was warmed up to room temperature, extracted with ethyl acetate; the organic phases were combined, washed with saturated sodium chloride solution, dried with anhydrous sodium sulfate, filtered, and the filtrate was concentrated under reduced pressure and purified to give 43.5 g of Compound 3.

NMR data of Compound 3: 1H NMR (90 MHz, Chloroform-d3) δ8.08 (s, 1H), 7.23-7.13 (m, 2H), 6.85-6.75 (m, 2H), 5.22 (s, 2H), 3.72 (s, 3H).

### 3. Synthesis of Compound 4

Compound 3 (43.5 g, 0.16 mmol) and L-serine ethyl ester hydrochloride (83.2 g, 0.49 mmol) were dissolved in a mixed solvent of 600 mL of ethanol and 600 mL of water, followed by the addition of sodium bicarbonate (110 g, 1.31 mol), and the reaction solution was heated up to 90° C. and stirred for 72 hours. After completion of the reaction, the reaction solution was cooled to room temperature, concentrated under reduced pressure to remove ethanol, diluted with dichloromethane, partitioned, and the aqueous phase was added with concentrated hydrochloric acid dropwise to pH<4, then extracted with ethyl acetate; the organic phases of ethyl acetate were combined, washed with saturated sodium chloride solution, dried with anhydrous sodium sulfate, filtered, and the filtrate was concentrated under reduced pressure and purified to give 49 g of compound 4. Ms [M+Na]+ 359.1

NMR data of Compound 4: 1H NMR (400 MHz, DMSO-d6) δ9.78(s, 1H), 7.17-7.14(d, J=8.91 Hz, 2H), 6.88-6.86(d, J=8.39 Hz, 2H), 6.82(s, 1H), 6.06-6.05(d, J=3.68 Hz, 1H), 5.04-5.03(d, J=2.55 Hz, 2H), 4.89-4.87(t, 1H), 3.80-3.76(q, 1H), 3.72(s, 3H), 3.60-3.57(t, 2H).

### 4. Synthesis of Compound 5

Compound 4 (8.5 g, 25.3 mmol) was dissolved in 340 mL of acetic acid, reduced iron powder (14.1 g, 253 mmol) was added, and the reaction was heated to 50° C. for 6 h under nitrogen protection. After completion of the reaction, the reaction solution was cooled to room temperature, diluted with ethyl acetate, filtered through diatomaceous earth, and the filtrate was concentrated under reduced pressure to evaporate the solvent; the residue was adjusted to pH>7 with saturated sodium bicarbonate, and then extracted with ethyl acetate. The organic phases were combined, washed with saturated saline, dried with anhydrous sodium sulfate, and concentrated under reduced pressure to give 4.4 g of crude compound 5. Ms [M+H]+ 289.1

NMR data of Compound 5: 1H NMR (400 MHz, DMSO-d6) δ9.80(s, 1H), 7.16-7.14(d, J=9.03 Hz, 2H), 6.88-6.86(d, J=8.53 Hz, 2H), 6.82(s, 1H), 6.08-6.07(d, J=3.51 Hz, 1H), 5.04-5.03(d, J=2.23 Hz, 2H), 4.92-4.89(t, 1H), 3.80-3.77(q, 1H), 3.72(s, 3H), 3.60-3.57(t, 2H).

### 5. Synthesis of Compound 6

The crude compound 5 (3.47 g, 12 mmol) was dissolved in 25 mL of N,N-dimethylformamide, imidazole (2.47 g, 36 mmol) was added, and tert-butyldiphenylchlorosilane (9.9 g, 36 mmol) was added dropwise under cooling of an ice-water bath and under nitrogen protection. After the addition was completed, the reaction solution was gradually warmed up to room temperature for 1 hour. After completion of the reaction, the reaction system was diluted with saturated sodium chloride solution, and extracted with ethyl acetate; the organic phases were combined, dried with anhydrous sodium sulfate, filtered, and the filtrate was concentrated under reduced pressure and purified to yield 5.4 g of compound 6. Ms [M+H]+ 527.4

NMR data of Compound 6: 1H NMR (400 MHz, Chloroform-d3) δ7.96 (s, 1H), 7.60-7.56 (m, 4H), 7.47-7.40 (m, 2H), 7.39-7.35 (m, 4H), 7.13-7.11 (m, 2H), 7.06 (s, 1H), 6.80 (d, J=8.6 Hz, 2H), 4.09-4.04 (m, 1H), 3.96-3.91 (m, 3H), 3.72 (s, 3H), 2.92 (d, J=28.3 Hz, 2H), 0.99 (s, 9H).

### 6. Synthesis of Compound 7

Compound 6 (5.4 g, 10.24 mmol) was dissolved in 70 mL of dioxane, manganese dioxide (4.47 g, 51.35 mmol) was added, and the reaction was heated to 35° C. for 1 hour. After completion of the reaction, the reaction solution was cooled to room temperature and filtered through diatomaceous earth; the filtrate was concentrated under reduced pressure to give 4.95 g of compound 7. Ms [M+H]+ 525.2

NMR data of Compound 7: 1H NMR (400 MHz, Chloroform-d3) δ7.78-7.71(m, 4H), 7.48(s, 1H), 7.42-7.36(m, 2H), 7.36-7.28(m, 6H), 6.84-6.77(m, 2H), 5.48(s, 2H), 5.04(s, 2H), 3.76(s, 3H), 1.16(s, 9H).

### 7. Synthesis of Compound 8

Compound 7 (5 g, 9.54 mmol) was dissolved in 45 mL of N,N-dimethylformamide, and then triethylamine (5.4 mL, 38 mmol) and N-phenylbis(trifluoromethanesulfonyl)imide (6.8 g, 19 mmol) were added sequentially under cooling of an ice-water bath, and then the reaction solution was moved to room temperature, and stirred for 1 hour. After completion of the reaction, the reaction solution was diluted with saturated saline, and then extracted with ethyl acetate for 3 times; the organic phases were combined, dried with anhydrous sodium sulfate, concentrated under reduced pressure and purified to give 8 g of compound 8.

### 8. Synthesis of Compound 9

Compound 8 (3.9 g, 5.87 mmol) was dissolved in 70 mL of acetonitrile, then N,N-diisopropylethylamine (10 mL, 53 mmol) and compound A trifluoroacetate (1.46 g, 5.3 mmol) were added, and the reaction solution was stirred at 75° C. under nitrogen protection for 20 hours. After the reaction was completed, the reaction solution was cooled to room temperature, and concentrated under reduced pressure; the residue was dissolved in ethyl acetate, washed with saturated saline. The organic phase was separated and dried with anhydrous sodium sulfate, concentrated under reduced pressure and purified to give 3.2 g of compound 9. Ms [M+H]+ 781.4

NMR data of Compound 9: 1H NMR (400 MHz, DMSO-d6) δ8.17(s, 1H), 7.75-7.73(m, J=8.0 Hz, 4 H), 7.44-7.37 (m, 6H), 7.21(dd, J=8.8, 2.2 Hz, 2H), 6.85-6.78(m, 2H), 5.51(s, 2H), 5.11(d, J=10.8 Hz, 1H), 4.97(s, 2H), 4.16-4.10(m, 1H), 3.78(d, J=8.6 Hz, 1H), 3.69(s, 3H), 3.48(d, J=8.6 Hz, 1H), 3.43-3.39(m, 1H), 3.26-3.22(m, 2H), 2.83(m, 2H), 1.94-1.79(m, 2H), 1.60(m, 2H), 1.18(s, 9H), 1.10(d, J=6.4 Hz, 3H), 1.01(s, 9H).

### 9. Synthesis of Compound 10

Compound 9 (1.4 g, 1.79 mmol) was dissolved in 30 mL of anhydrous tetrahydrofuran and 1 mol/L solution of tetrabutylammonium fluoride in tetrahydrofuran (2.7 mL, 2.7 mmol) was added under cooling of an ice-water bath, and then the reaction was moved to room temperature and stirred for 2 hours. After completion of the reaction, the reaction was diluted with ethyl acetate, washed with saturated saline, and the organic phase was separated and dried with anhydrous sodium sulfate, concentrated under reduced pressure and purified to give 830 mg of compound 10. Ms [M+H]+ 543.4

NMR data of Compound 10: 1H NMR (400 MHz, DMSO-d6) δ8.17(s, 1H), 7.28-7.20(m, 2H), 6.91-6.83(m, 2H), 5.58(s, 2H), 5.42(s, 1H), 5.14(d, J=10.8 Hz, 1H), 4.71(s, 2H), 4.16-4.10 (m, 1H), 3.82(d, J=8.6 Hz, 1H), 3.70(s, 3H), 3.52(d, J=8.6 Hz, 1H), 3.46-3.42(m, 3H), 2.85-2.84(m, 2H), 1.97-1.90(m, 2H), 1.66(t, J=16.5 Hz, 2H), 1.18(s, 9H), 1.11(d, J=6.4 Hz, 3H).

### 10. Synthesis of Compound 11

Compound 10 (1.5 g, 2.77 mmol) was dissolved in 40 mL of dichloromethane, triethylamine (0.78 mL, 5.54 mmol) was added, then tert-butylsulfinyl chloride (584 mg, 4.16 mmol in 2 mL of dichloromethane) was added dropwise under cooling of an ice-water bath. After addition, the reaction was moved to room temperature and stirred for 1 h. After completion of the reaction, the reaction was quenched with water, and then the reaction solution was extracted with dichloromethane; the organic phases were combined, dried with anhydrous sodium sulfate, concentrated under reduced pressure and purified to give 1.26 g of compound 11. Ms [M+H]+ 647.4

NMR data of Compound 11: 1H NMR (400 MHz, DMSO-d6) δ8.25(s, 1H), 7.27-7.17(m, 2H), 6.90-6.80(m, 2H), 5.56(s, 2H), 5.37-5.27(m, 2H), 5.15(d, J=10.5 Hz, 1H), 4.16-4.10(m, 1H), 3.82(d, J=8.6 Hz, 1H), 3.70(s, 3H), 3.57-3.48(m, 1H), 3.48-3.42(m, 1H), 3.29-3.21(m, 2H), 2.95-2.81(m, 2H), 1.99-1.92(m, 2H), 1.71-1.62(m, 2H), 1.18(s, 9H), 1.14(s, 9H), 1.11(d, J=6.3 Hz, 3H).

### 11. Synthesis of Compound B

Compound 11 (1.26 g, 1.86 mmol) was dissolved in 12 mL of trifluoroacetic acid under ice-water bath, the reaction system was replaced with nitrogen for three times, then trifluoromethanesulfonic acid (1.2 mL) was added under cooling of an ice-water bath, and the reaction was moved to room temperature and stirred for 2.5 hours. After completion of the reaction, saturated sodium bicarbonate aqueous solution was added under cooling of an ice-water bath to adjust pH to 8. The reaction solution was extracted with ethyl acetate for three times, and the organic phases were combined, dried with anhydrous sodium sulfate, concentrated under reduced pressure and purified to give 700 mg of compound B. Ms [M+H]+ 527.4

NMR data of Compound B: 1H NMR (400 MHz, DMSO-d6) δ8.23(s, 1H), 5.29-5.23(m, 2H), 5.15(d, J=10.7 Hz, 1H), 4.19-4.13(m, 1H), 3.85-3.80(m, 1H), 3.55-3.51(m, 1H), 3.49-3.45(m, 1H), 3.32-3.23(m, 2H), 2.95-2.85(m, 2H), 1.98-1.93(m, 2H), 1.72-1.64(m, 2H), 1.18(s, 9H), 1.14(s, 9H), 1.11(d, J=6.4 Hz, 3H).

### EXAMPLE 1

The compound synthesized in the present invention:

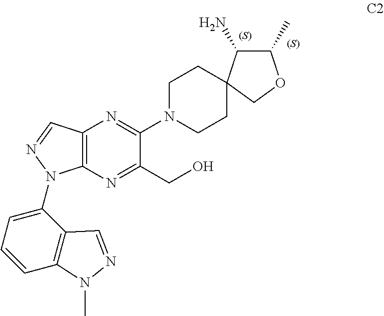

The synthetic route of compound C2 is as follows:

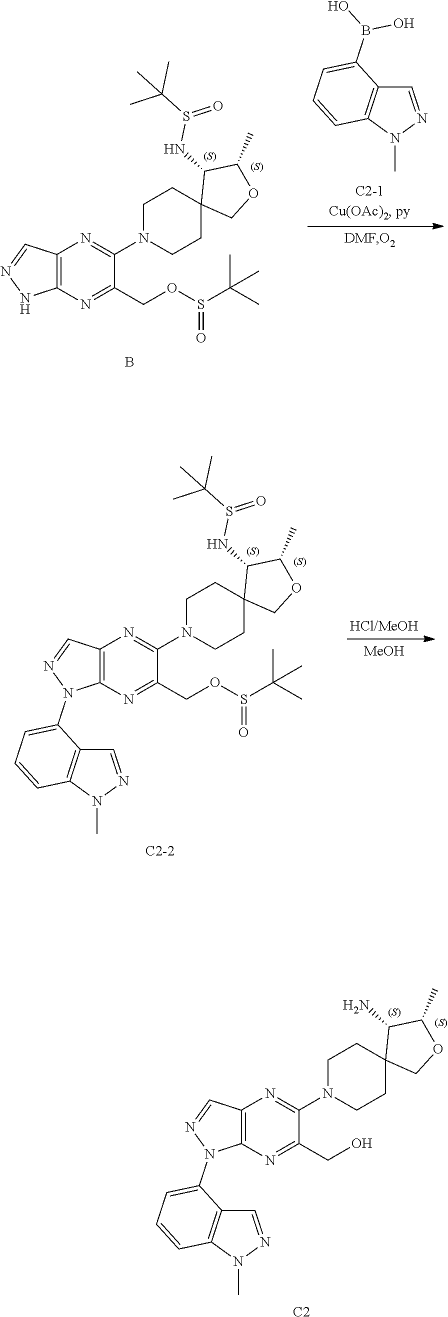

### 1. Synthesis of C2-2

Compound B (90 mg, 0.17 mmol) was dissolved in 5 mL of N,N-dimethylformamide, compound C2-1 (60 mg, 0.34 mmol), copper acetate (31 mg, 0.17 mmol) and pyridine (27 mg, 0.34 mmol) were added sequentially and the reaction solution was stirred at 25° C. for 3 hrs under oxygen protection. After completion of the reaction, the reaction was diluted with ethyl acetate, washed with saturated sodium chloride, and the organic phase was separated and dried with anhydrous sodium sulfate, concentrated under reduced pressure and purified to give 60 mg of compound C2-2. Ms [M+H]+ 657.5

### 2. Synthesis of C2

Compound C2-2 (60 mg, 0.09 mmol) was dissolved in 2 mL of methanol, and then 5 mL of hydrochloric acid methanol was added. The reaction was stirred at 25° C. for 30 min under nitrogen protection, and then warmed up to 55° C. and stirred for 5 h. After completion of the reaction, the reaction was cooled to room temperature, concentrated under reduced pressure and purified to give 5 mg of compound C2 trifluoroacetate. Ms [M+H]+ 449.4

NMR data of Compound C2: 1H NMR (400 MHz, Methanol-d4) δ8.52(s, 1H), 8.34(d, J=7.9 Hz, 1H), 8.33(s, 1 H), 7.57-7.48(m, 2H), 4.84(s, 2H), 4.27-4.22(m, 1H), 4.09(s, 3H), 3.84(d, J=8.6 Hz, 1H), 3.71(d, J=8.7 Hz, 1H), 3.44-3.39(m, 2H), 3.13-2.95(m, 3H), 1.97-1.89(m, 2H), 1.79-1.75(m, 2H), 1.20(d, J=6.5 Hz, 3H).

### EXAMPLE 2

The compound synthesized in the present invention:

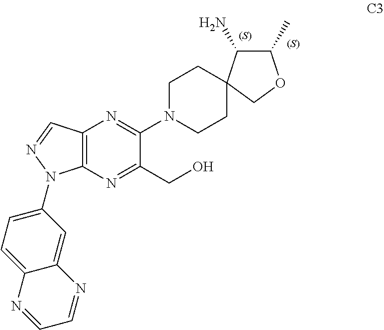

The synthetic route of compound C3 is as follows:

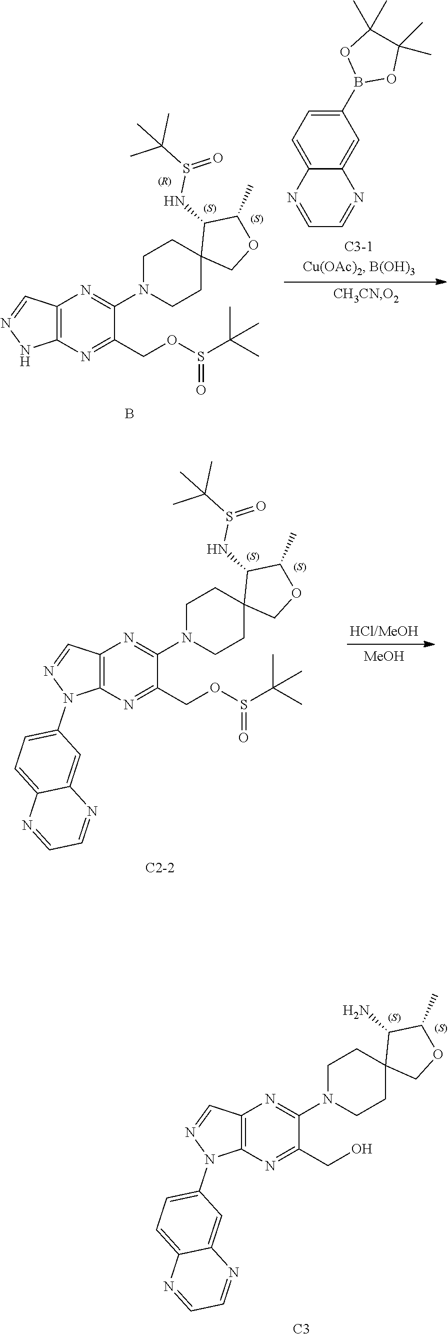

### 1. Synthesis of Compound C3-2

To a solution of compound B (100 mg, 0.19 mmol) dissolved in 5 mL of acetonitrile was added sequentially compound C3-1 (146 mg, 0.57 mmol), copper acetate (35 mg, 0.19 mmol), and boric acid (23 mg, 0.38 mmol)and then stirred at 80° C. under oxygen protection overnight. After completion of the reaction, the reaction solution was diluted with ethyl acetate, washed with saturated sodium chloride solution, and the organic phases were combined, dried with anhydrous sodium sulfate, concentrated under reduced pressure and purified to give 35 mg of compound C3-2. Ms [M+H]+ 655.3

### 2. Synthesis of Compound C3

Compound C3-2 (35 mg, 0.054 mmol) was dissolved in 1.5 mL of methanol, then 3.5 mL of 4M HCl methanol solution was added, and the reaction solution was stirred at 25° C. for 30 min under nitrogen protection, and then warmed up to 55° C. for 3 hours. After completion of the reaction, the reaction solution was cooled to room temperature, concentrated under reduced pressure and purified to give 3 mg of compound C3 trifluoroacetate. Ms [M+H]+ 447.2

NMR data of Compound C3: 1H NMR (400 MHz, Methanol-d4) δ9.28(d, J=2.4 Hz, 1H), 9.01(dd, J=9.3, 2.4 Hz, 1H), 8.95(d, J=1.9 Hz, 1H), 8.91(d, J=1.9 Hz, 1H), 8.39(s, 1H), 8.28(d, J=9.3 Hz, 1H), 4.96(s, 2H), 4.35-4.31(m, 1H), 4.00(d, J=9.1 Hz, 1H), 3.90(d, J=9.1 Hz, 1H), 3.67-3.56(m, 2H), 3.52-3.49(m, 1H), 3.14-3.02(m, 2H), 2.10-2.04(m, 2H), 1.98(d, J=13.3 Hz, 1H), 1.81(d, J=12.8 Hz, 1H), 1.35(d, J=6.5 Hz, 3H).

### EXAMPLE 3

The compound synthesized in the present invention:

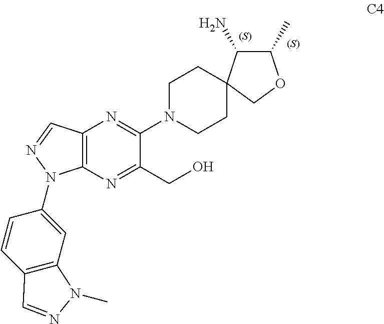

The synthetic route of compound C4 is as follows:

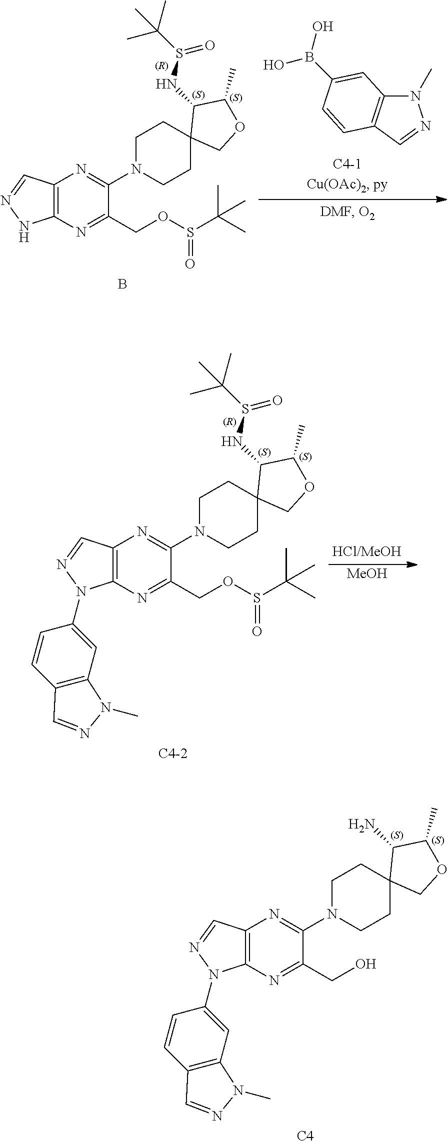

### 1. Synthesis of Compound C4-2

To a solution of compound B (80 mg, 0.15 mmol) in 6 mL of N,N-dimethylformamide was added sequentially compound C4-1 (54 mg, 0.30 mmol), copper acetate (27 mg, 0.15 mmol) and pyridine (24 mg, 0.30 mmol)and then stirred at 25° C. for 5 hrs under oxygen protection. After completion of the reaction, the reaction solution was diluted with ethyl acetate, and washed with saturated sodium chloride solution; the organic phase was separated and dried with anhydrous sodium sulfate, concentrated under reduced pressure and purified to give 70 mg of compound C4-2. Ms [M+H]+ 657.5

### 2. Synthesis of Compound C4

To a solution of Compound C4-2 (70 mg, 0.11 mmol) in 4 mL of methanol was added 6 mL of 4M HCl methanol solution, then stirred at 25° C. for 30 min under nitrogen protection, and then warmed up to 55° C. for 4 hours. After reaction, the reaction solution was cooled to room temperature, concentrated under reduced pressure and purified to give 8 mg of compound C4 trifluoroacetate. Ms [M+H]+ 449.2

NMR data of Compound C4: 1H NMR (400 MHz, Methanol-d4) δ8.55(s, 1H), 8.33(s, 1H), 8.22(dd, J=8.8, 1.8 Hz, 1H), 8.08(d, J=1.0 Hz, 1H), 7.93(dd, J=8.8, 0.7 Hz, 1H), 4.95(s, 2H), 4.37-4.32(m, 1H), 4.17(s, 3H), 4.00(d, J=12 Hz, 1H), 3.90(d, J=12 Hz, 1H), 3.65-3.51(m, 3H), 3.13 -3.01(m, 2H), 2.11-1.97(m, 3H), 1.83-1.79(m, 1H), 1.36(d, J=6.5 Hz, 3H).

### EXAMPLE 4

The compound synthesized in the present invention:

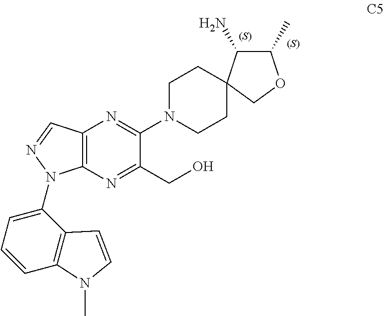

The synthetic route of compound C5 is as follows:

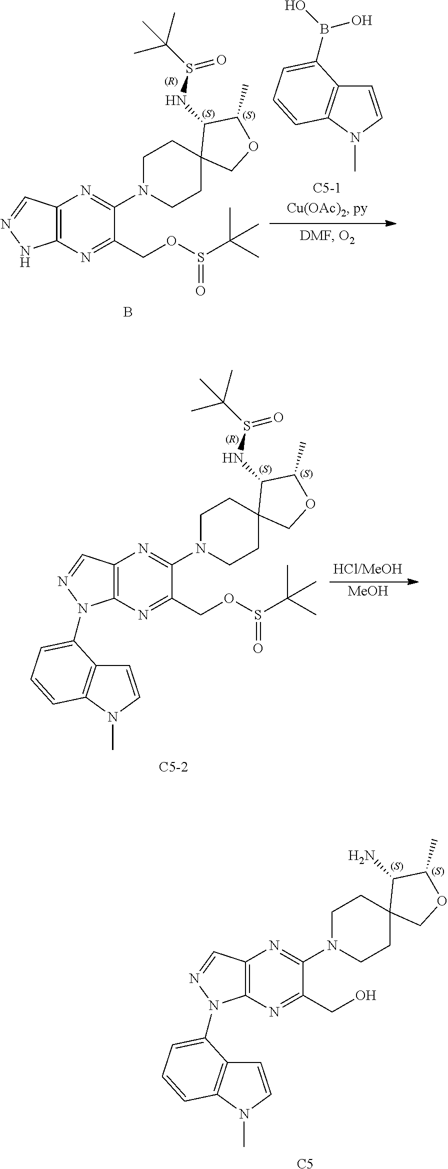

### 1. Synthesis of Compound C5-2

To a solution of Compound B (100 mg, 0.19 mmol) in 5 mL of N,N-dimethylformamide was added sequentially compound C5-1 (67 mg, 0.38 mmol), copper acetate (35 mg, 0.19 mmol) and pyridine (30 mg, 0.38 mmol), and then stirred at 25° C. for 2 hrs under oxygen protection. After completion of the reaction, the reaction solution was diluted with ethyl acetate, and washed with saturated sodium chloride solution; the organic phases were combined, dried with anhydrous sodium sulfate, concentrated under reduced pressure and purified to give 76 mg of compound C5-2. Ms [M+H]+ 656.4

### 2. Synthesis of Compound C5

Compound C5-2 (70 mg, 0.11 mmol) was dissolved in 4 mL of methanol, followed by the addition of 4M HCl methanol solution. The reaction was stirred at 25° C. for 30 min under nitrogen protection, and then warmed up to 55° C. for 5 hours. After completion of the reaction, the reaction solution was cooled to room temperature, spin-dried, concentrated and purified to give 3 mg of compound C5 trifluoroacetate. Ms [M+H]+ 448.3

NMR data of Compound C5: ¹H NMR (400 MHz, Methanol-d4) δ8.33(s, 1H), 7.68(dd, J=7.7, 0.8 Hz, 1H), 7.54-7.51(m, 1H), 7.38(t, J=7.8 Hz, 1H), 7.27(d, J=3.2 Hz, 1H), 6.63(dd, J=3.2, 0.9 Hz, 1H), 4.86(s, 2H), 4.35-4.33(m, J=6.5, 4.1 Hz, 1H), 4.00(d, J=9.1 Hz, 1H), 3.91-3.89(m, 4H), 3.63-3.48(m, 3H), 3.13-2.98(m, 2H), 2.10-2.04(m, 2H), 1.99-1.95(m, 1H), 1.82-1.79(m, 1H), 1.36(d, J=6.5 Hz, 3H).

### EXAMPLE 5

The compound synthesized in the present invention:

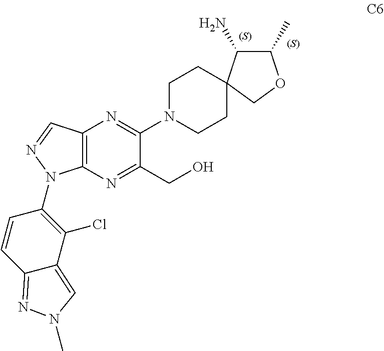

The synthetic route of compound C6 is as follows:

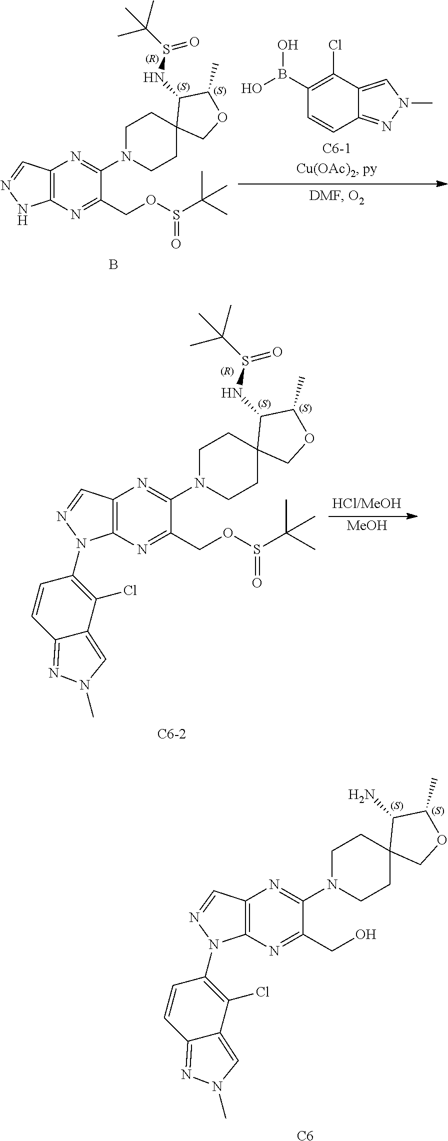

### 1. Synthesis of Compound C6-2

To a solution of Compound B (100 mg, 0.19 mmol) in 5 ml of N,N-dimethylformamide was added sequentially compound C6-1 (120 mg, 0.57 mmol), copper acetate (17 mg, 0.095 mmol) and pyridine (45 mg, 0.57 mmol) and stirred at 25° C. for 1 hr under oxygen protection. After completion of the reaction, the reaction was diluted with ethyl acetate, washed with saturated sodium chloride, dried with anhydrous sodium sulfate, concentrated under reduced pressure and purified to give 80 mg of compound C6-2. Ms [M+H]+ 691.3

### 2. Synthesis of Compound C6

Compound C6-2 (80 mg, 0.12 mmol) was dissolved in 3 mL of methanol, and then 5 mL of 4M HCl methanol solution was added; the reaction solution was stirred at 25° C. for 30 min under nitrogen protection, and then warmed up to 55° C. for 4 hours. After completion of the reaction, the reaction solution was cooled to room temperature, concentrated under reduced pressure and purified to give 20 mg of compound C6 trifluoroacetate. Ms [M+H]+ 483.2

NMR data of Compound C6: 1H NMR (400 MHz, Methanol -d4) δ8.52 (s, 1H), 8.34 (s, 1H), 7.74 (dd, J=9.0, 0.9 Hz, 1H), 7.44 (d, J=8.9 Hz, 1H), 4.82 (s, 2H), 4.38-4.32 (m, 4H), 4.00 (d, J=9.1 Hz, 1H), 3.89 (d, J=9.2 Hz, 1H), 3.62 -3.49 (m, 3H), 3.11-2.98 (m, 2H), 2.05 (d, J=9.5 Hz, 2H), 1.98-1.95 (m, 1H), 1.82-1.79 (m, 1H), 1.35 (d, J=6.5 Hz, 3H).

### EXAMPLE 6

The compound synthesized in the present invention:

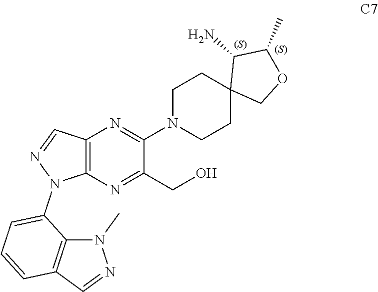

The synthetic route of compound C7 is as follows:

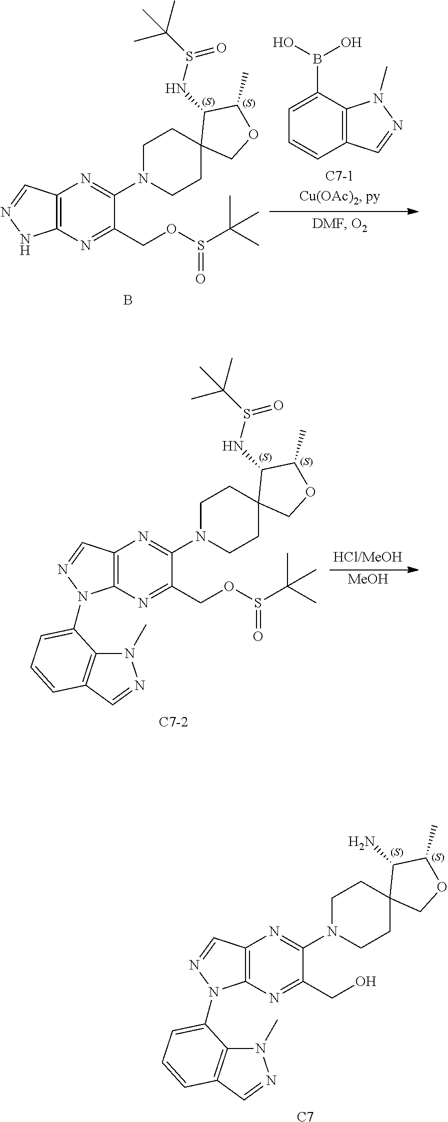

### 1. Synthesis of Compound C7-2

To a solution of Compound B (70 mg, 0.13 mmol) in 5 mL of N,N-dimethylformamide was added sequentially compound C7-1 (49 mg, 0.26 mmol), copper acetate (28 mg, 0.13 mmol) and pyridine (21 mg, 0.26 mmol)and stirred at 25° C. for 3 hrs under oxygen atmosphere. After completion of the reaction, the reaction solution was diluted with ethyl acetate, and washed with saturated sodium chloride solution; the organic phase was separated and dried with anhydrous sodium sulfate, concentrated under reduced pressure and purified to give 30 mg of compound C7-2. Ms [M+H]+ 657.4

### 2. Synthesis of Compound C7

Compound C7-2 (30 mg, 0.046 mmol) was dissolved in 2 mL of methanol, and then 4 mL of 4M HCl methanol solution was added; the reaction solution was stirred at 25° C. for 30 min under nitrogen protection, and then warmed up to 55° C. for 6 hours. After completion of the reaction, the reaction solution was cooled to room temperature, concentrated under reduced pressure and purified to give 3 mg of compound C7 trifluoroacetate. Ms [M+H]+ 449.2

NMR data of Compound C7: 1H NMR (400 MHz, Methanol-d4) 8 8.39 (s, 1H), 8.15 (s, 1H), 7.98 (dd, J=8.2, 1.0 Hz, 1H), 7.51 (dd, J=7.3, 1.0 Hz, 1H), 7.32 (dd, J=8.1, 7.3 Hz, 1H), 4.78 (s, 2H), 4.35-4.29 (m, 1H), 3.98 (d, J=9.1 Hz, 1H), 3.87 (d, J=9.1 Hz, 1H), 3.62-3.47 (m, 3H), 3.42 (s, 3H), 3.10-2.97 (m, 2H), 2.09-2.00 (m, 2H), 1.96-1.93 (m, 1H), 1.80-1.77 (m, 1H), 1.33 (d, J=6.5 Hz, 3H).

### EXAMPLE 7

The compound synthesized in the present invention:

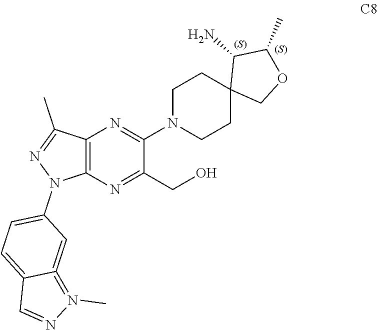

The synthetic route of compound C8 is as follows:

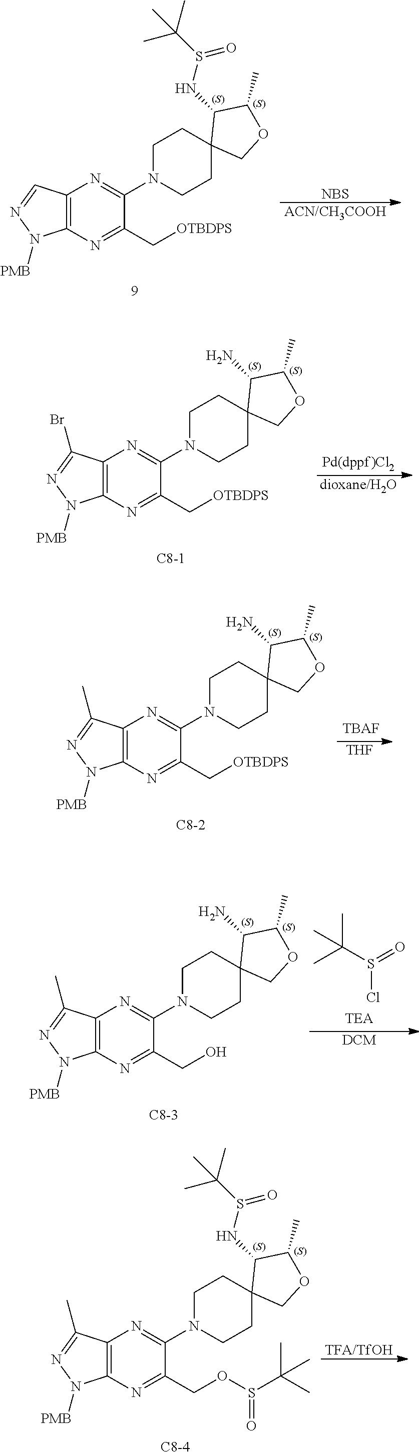

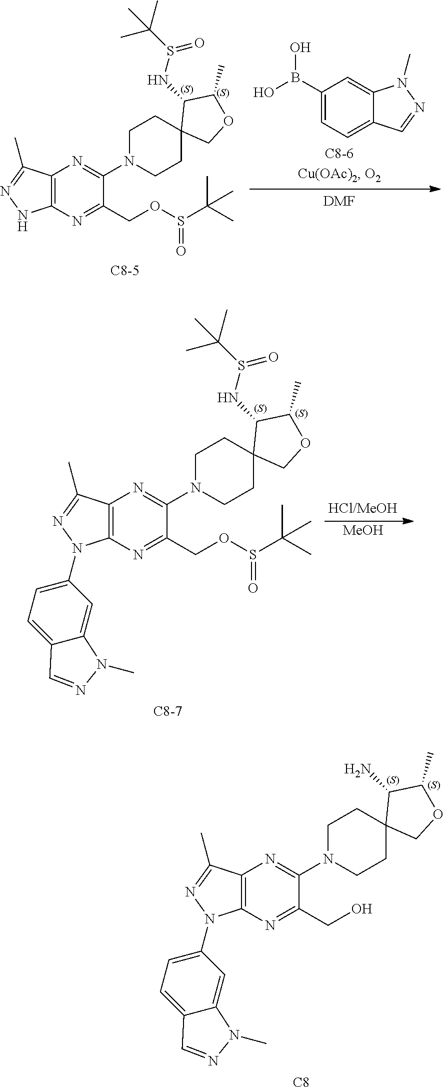

### 1. Synthesis of Compound C8-1

Compound 9 (700 mg, 0.90 mmol) was dissolved in a mixed solvent of 8 mL of acetonitrile and 8 mL of acetic acid, and N-bromosuccinimide (480 mg, 2.69 mmol) was added in batches under cooling of an ice-salt bath, and the reaction was kept at the temperature under stirring for 30 min. The reaction was then moved to room temperature and stirred for 2 h. After completion of the reaction, the reaction was quenched with water, and extracted with ethyl acetate; the organic phases were combined, dried with anhydrous sodium sulfate, concentrated under reduced pressure and purified to give 500 mg of compound C8-1. Ms [M+H]+ 755.4

### 2. Synthesis of Compound C8-2

To a solution of Compound C8-1 (500 mg, 0.66 mmol) in a mixed solvent of 1,4-dioxane and water (10 mL/1 mL) was added sequentially methylboronic acid (120 mg, 1.99 mmol), 1,1′-bis(diphenylphosphino)ferrocene palladium(II) chloride, dichloromethane complex (54 mg, 0.66 mmol), and sodium carbonate (210 mg, 1.98 mmol), then the reaction solution was stirred at 100° C. for 10 h under nitrogen protection. After completion of the reaction, the reaction solution was cooled to room temperature, quenched with water, and extracted with ethyl acetate; the organic phases were combined, dried with anhydrous sodium sulfate, concentrated under reduced pressure and purified to give 270 mg of compound C8-2. Ms [M+H]+ 691.5

### 3. Synthesis of compound C8-3

Compound C8-2 (270 mg, 0.39 mmol) was dissolved in 5 mL of tetrahydrofuran and 1 M solution of tetrabutylammonium fluoride in tetrahydrofuran (0.47 mL, 0.47 mmol) was added under an ice-water bath, and the reaction was then moved to room temperature and stirred for 2 hours. After completion of the reaction, the reaction solution was directly concentrated under reduced pressure and purified to give 180 mg of compound C8-3.

NMR data of Compound C8-3: 1H NMR (400 MHz, DMSO-d6) δ7.25-7.18 (m, 2H), 6.89-6.83 (m, 2H), 5.49 (s, 2H), 5.36 (t, J=5.9 Hz, 1H), 4.70 (d, J=5.8 Hz, 2H), 4.11-4.04 (m, 1H), 3.70 (s, 4H), 3.51 (d, J=8.3 Hz, 1H), 3.46-3.40 (m, 1H), 3.28-3.20 (m, 2H), 3.02-2.89 (m, 2H), 2.44 (s, 3H), 1.91-1.76 (m, 2H), 1.68-1.53 (m, 2H), 1.10 (d, J=6.5 Hz ,3H).

### 4. Synthesis of Compound C8-4

Compound C8-3 (200 mg, 0.44 mmol) was dissolved in 6 mL of dichloromethane, triethylamine (0.2 mL, 1.32 mmol) was added, and then a solution of tert-butylsulfinyl chloride in dichloromethane(160 mg, 1.1 mmol in 3 mL of dichloromethane) was added dropwise under cooling of an ice-water bath. After addition, the reaction solution was moved to room temperature and stirred for 1 h. After completion of the reaction, the reaction was quenched with water, and extracted with dichloromethane; the organic phases were combined, dried with anhydrous sodium sulfate, concentrated under reduced pressure and purified to give 180 mg of compound C8-4. Ms [M+H]+ 661.4

### 5. Synthesis of Compound C8-5

Compound C8-4 (180 mg, 0.27 mmol) was dissolved in 2 mL of trifluoroacetic acid under an ice-water bath, and trifluoromethanesulfonic acid (0.2 mL) was added dropwise under cooling of an ice-water bath. After addition, the reaction solution was moved to room temperature and stirred for 6.5 h. After completion of the reaction, under cooling of an ice-water bath, the reaction solution was added dropwise with saturated sodium bicarbonate aqueous solution, adjusted to pH 8, and extracted with ethyl acetate; the organic phases were combined, dried with anhydrous sodium sulfate, and then concentrated under reduced pressure and purified to give 90 mg of compound C8-5. Ms [M+H]+ 541.3

### 6. Synthesis of Compound C8-7

To a solution of Compound C8-5 (90 mg, 0.16 mmol) in 5 mL of N,N-dimethylformamide was added sequentially compound C8-6 (60 mg, 0.34 mmol), copper acetate (31 mg, 0.17 mmol) and pyridine (27 mg, 0.34 mmol)and stirred at 25° C. for 2 hrs under oxygen protection. After completion of the reaction, the reaction solution was diluted with ethyl acetate, and extracted with saturated sodium chloride solution; the organic phases were combined, dried with anhydrous sodium sulfate, concentrated under reduced pressure and purified to give 70 mg of compound C8-7. Ms [M+H]+ 671.4

### 7. Synthesis of Compound C8

Compound C8-7 (70 mg, 0.10 mmol) was dissolved in 3 mL of methanol, and then 6 mL of 4M HCl methanol solution was added; the reaction solution was stirred at 25° C. for 30 min under nitrogen protection, and then warmed up to 55° C. for 7 hours. After completion of the reaction, the reaction was cooled to room temperature, concentrated under reduced pressure and purified to give 3 mg of compound C8 trifluoroacetate. Ms [M+H]+ 463.2

NMR data of Compound C8: 1H NMR (400 M , Methanol-d4) δ8.46 (s, 1H), 8.20 (dd, J=8.8, 1.8 Hz, 1H), 8.03 (d, J=1.0 Hz, 1H), 7.88 (d, J=8.9 Hz, 1H), 4.92 (s, 2H), 4.36-4.29 (m, 1H), 4.14 (s, 3H), 3.99 (d, J=9.1 Hz, 1H), 3.89 (d, J=9.2 Hz, 1H), 3.60-3.46 (m, 3H), 3.10-2.98 (m, 2H), 2.65 (s, 3H), 2.10-2.01 (m, 2H), 1.98-1.95 (m, 1H), 1.81-1.78 (m , 1H), 1.34 (d, J=6.5 Hz, 3H).

### EXAMPLE 8

The compound synthesized in the present invention:

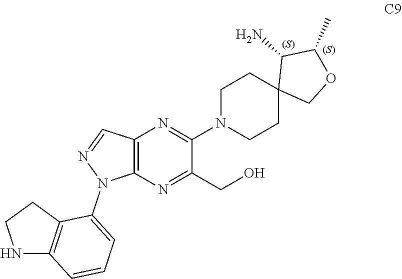

The synthetic route of compound C9 is as follows:

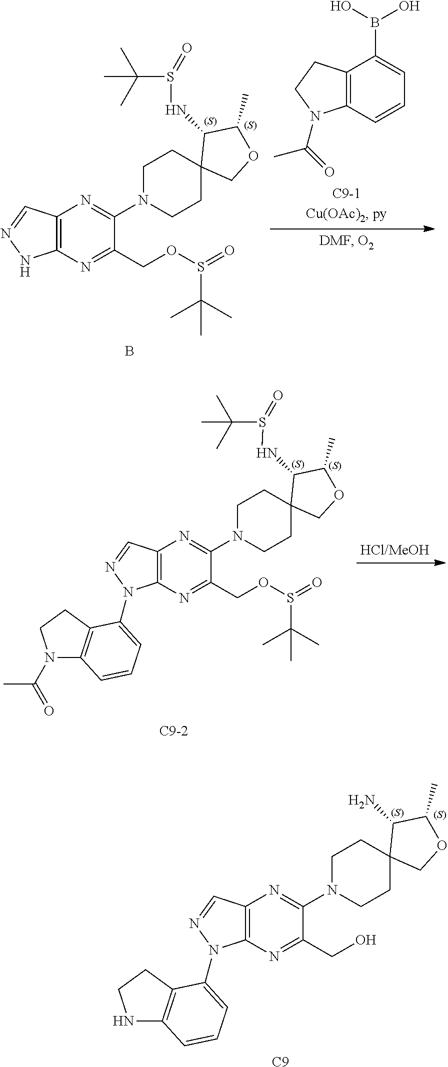

### 1. Synthesis of Compound C9-2

To a solution of Compound B (90 mg, 0.17 mmol) in 5 mL of N,N-dimethylformamide was added sequentially compound C9-1 (70 mg, 0.34 mmol), copper acetate (31 mg, 0.17 mmol) and pyridine (27 mg, 0.34 mmol)and stirred at 25° C. for 3 hrs under oxygen atmosphere. After completion of the reaction, the reaction solution was diluted with ethyl acetate, and washed with saturated sodium chloride solution; the organic phases were combined, dried with anhydrous sodium sulfate, concentrated under reduced pressure and purified to give 130 mg of compound C9-2. Ms [M+H]+ 686.4

### 2. Synthesis of compound C9

Compound C9-2 (60 mg, 0.19 mmol) was dissolved in 3 mL of methanol, and then 4 mL of 4M HCl methanol solution was added; the reaction solution was stirred at 25° C. for 30 min under nitrogen protection, and then warmed up to 55° C. for 5 hours. After completion of the reaction, the reaction solution was cooled to room temperature, concentrated under reduced pressure and purified to give 7 mg of compound C9 trifluoroacetate. Ms [M+H]+ 436.3

NMR data of Compound C9: 1H NMR (400 MHz, Methanol-d4) δ8.30 (s, 1H), 7.92 (dd, J=8.1, 0.9 Hz, 1H), 7.50 (t, J=8.0 Hz, 1H), 7.30-7.27 (m, 1H), 4.33-4.29 (m, 1H), 4.00-3.96 (d, J=12 Hz, 1H), 3.88-3.84 (d, J=12 Hz, 1H), 3.79 (t, J=7.9 Hz, 2H), 3.62-3.45 (m, 5H), 3.09-2.97 (m, 2H), 2.09-2.03 (m, 2H), 1.96-1.93 (m, 1H), 1.80-1.76 (m, 1H), 1.34 (d, J=6.4 Hz, 3H).

### EXAMPLE 9

The compound synthesized in the present invention:

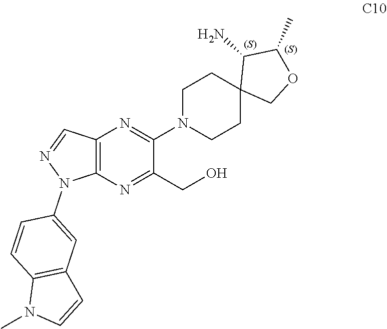

The synthetic route of compound C10 is as follows:

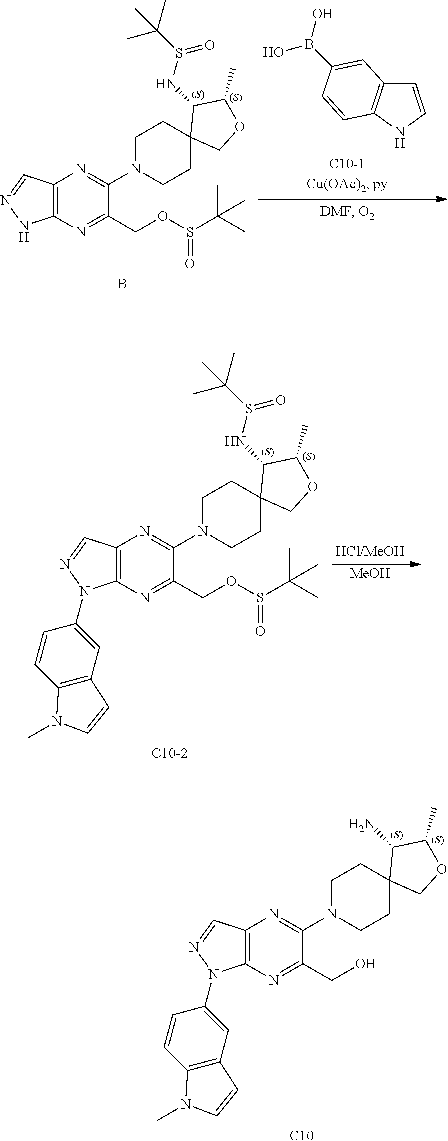

### 1. Synthesis of Compound C10-2

To a solution of Compound B (90 mg, 0.17 mmol) in 5 mL of N,N-dimethylformamide was added sequentially compound C10-1 (63 mg, 0.34 mmol), copper acetate (34 mg, 0.17 mmol) and pyridine (27 mg, 0.34 mmol)and stirred at 25° C. for 2 hrs under oxygen atmosphere. After completion of the reaction, the reaction was diluted with ethyl acetate, washed with saturated sodium chloride solution, dried with anhydrous sodium sulfate, concentrated under reduced pressure and purified to give 52 mg of compound C10-2. Ms [M+H]+ 656.4

### 2. Synthesis of Compound C10

Compound C10-2 (52 mg, 0.08 mmol) was dissolved in 2 mL of methanol, and then 2 mL of HCl methanol solution was added; the reaction solution was stirred at 25° C. for 30 min under nitrogen protection, and then warmed up to 55° C. for 8 hours. After completion of the reaction, the reaction solution was cooled to room temperature, concentrated under reduced pressure and purified to give 2.08 mg of compound C10 trifluoroacetate. Ms [M+H]+ 448.3

NMR data of Compound C10: 1H NMR (400 MHz, Methanol-d4) δ8.26 (d, J=2.0 Hz, 1H), 8.22 (s, 1H), 7.87 (dd, J=8.8, 2.1 Hz, 1H), 7.54 (d, J=8.8 Hz, 1H), 7.28 (s, 1H), 6.55 (d, J=3.1 Hz, 1H), 4.88 (s, 2H), 4.36-4.28 (m, 1H), 3.98 (d. J=9.2 Hz, 1H), 3.91-3.83 (m, 4H), 3.61-3.52 (m, 2H), 3.49-3.48 (m, 1H), 3.10-2.95 (m, 2H), 2.07-2.00 (m, 2H), 1.95 (d, J=12.9 Hz, 1H), 1.78 (d, J=12.8 Hz, 1H), 1.33 (d, J=6.5 Hz, 3H).

### EXAMPLE 10

The compound synthesized in the present invention:

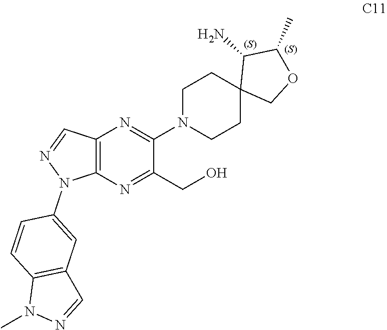

The synthetic route of compound C11 is as follows:

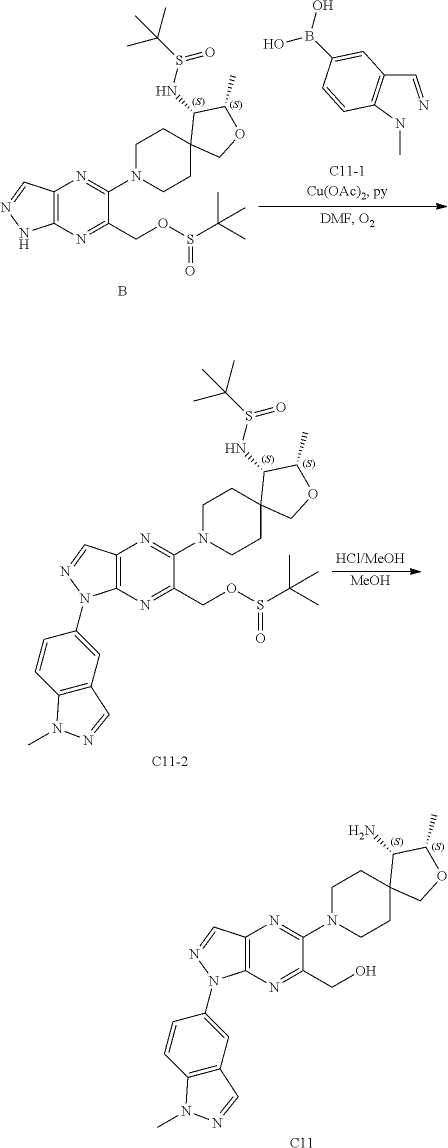

### 1. Synthesis of Compound C11-2

To a solution of Compound B (100 mg, 0.19 mmol) in 5 mL of N,N-dimethylformamide was added sequentially compound C11-1 (67 mg, 0.38 mmol), copper acetate (35 mg, 0.19 mmol) and pyridine (30 mg, 0.38 mmol) and stirred at 25° C. for 2 hrs under oxygen atmosphere. After completion of the reaction, the reaction was diluted with ethyl acetate, washed with saturated sodium chloride solution, dried with anhydrous sodium sulfate, concentrated under reduced pressure and purified to give 35 mg of compound C11-2. Ms [M+H]+ 657.4

### 1. Synthesis of Compound C11

Compound C11-2 (35 mg, 0.05 mmol) was dissolved in 2 mL of methanol, and then 2 mL of 4M HCl methanol solution was added; the reaction solution was stirred at 25° C. for 30 min under nitrogen protection, and then warmed up to 55° C. for 6 hours. After completion of the reaction, the reaction solution was cooled to room temperature, concentrated under reduced pressure and purified to give 8.32 mg of compound C11 trifluoroacetate. Ms [M+H]+ 449.2

NMR data of Compound C11: 1H NMR (400 MHz, Methanol-d4) δ8.65 (d, J=1.9 Hz, 1H), 8.31 (dd, J=9.1, 1.9 Hz, 1H), 8.27 (s, 1H), 8.13 (s, 1H), 7.75 (d, J=9.1 Hz, 1H), 4.92 (s. 2H), 4.37-4.31 (m, 1H), 4.15 (s, 3H), 4.00 (d, J=9.2 Hz, 1H), 3.89 (d, J=9.2 Hz, 1H), 3.64-3.47 (m, 3H), 3.11-2.99 (m, 2H), 2.92 (s, 3H), 2.11-2.05 (m, 2H), 1.97 (d, J=12.9 Hz, 1H), 1.81 (d, J=12.8 Hz, 1H), 1.36 (d, J=6.5 Hz, 3H).

### EXAMPLE 11

The compound synthesized in the present invention:

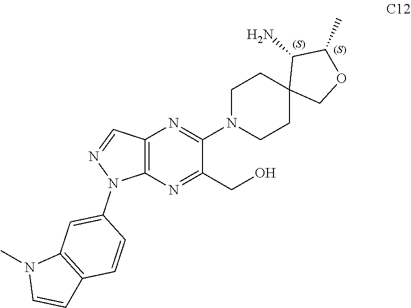

The synthetic route of compound C12 is as follows:

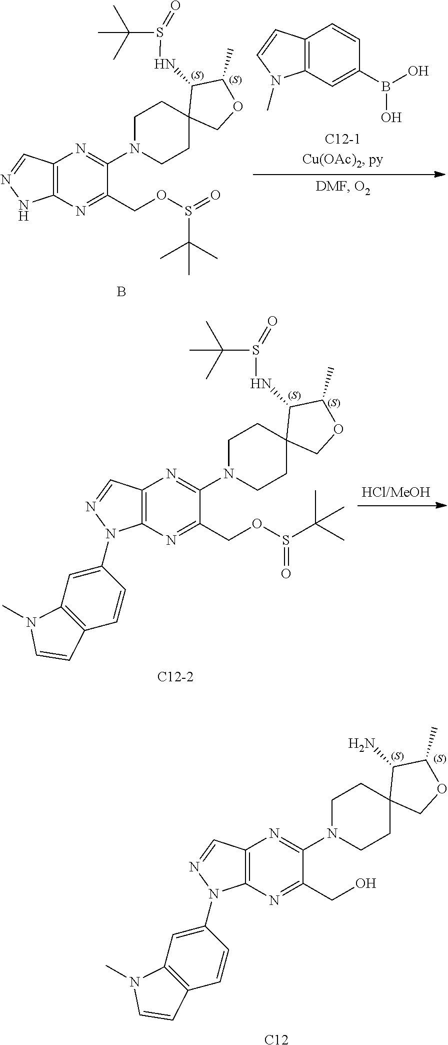

### 1. Synthesis of Compound C12-2

To a solution of Compound B (70 mg, 0.13 mmol) in 5 mL of N,N-dimethylformamide was added sequentially compound C12-1 (46 mg, 0.26 mmol), copper acetate (24 mg, 0.13 mmol) and pyridine (21 mg, 0.26 mmol) and stirred at 25° C. for 2 hrs under oxygen atmosphere. After completion of the reaction, the reaction solution was diluted with ethyl acetate, and washed with saturated sodium chloride solution; the organic phase was separated and dried with anhydrous sodium sulfate, concentrated under reduced pressure and purified to give 104 mg of compound C12-2. Ms [M+H]+ 656.4

### 2. Synthesis of Compound C12

Compound C12-2 (104 mg, 0.16 mmol) was dissolved in 5 mL of methanol, and then 5 mL of 4M HCl methanol solution was added; the reaction solution was stirred at 25° C. for 30 min under nitrogen protection, and then warmed up to 55° C. for 6 hours. After completion of the reaction, the reaction was cooled to room temperature, concentrated under reduced pressure and purified to give 1 mg of compound C12 trifluoroacetate. Ms [M+H]+ 448.2

### EXAMPLE 12

The compound synthesized in the present invention:

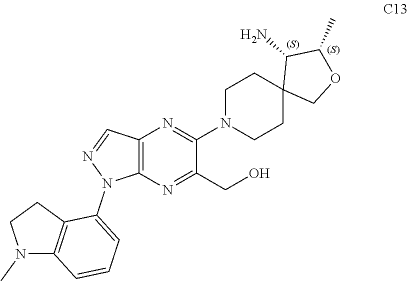

The synthetic route of compound C13 is as follows:

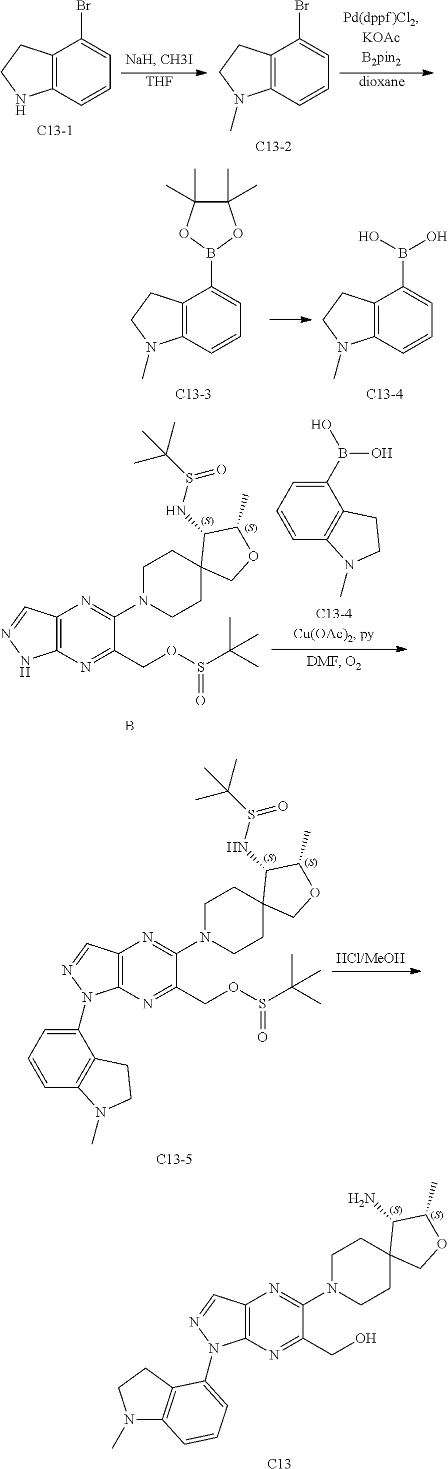

### 1. Synthesis of Compound C13-2

Compound C13-1 (2.4 g, 12.12 mmol) was dissolved in 90 mL of anhydrous tetrahydrofuran under the nitrogen protection and cooling of an ice-water bath, and 60% sodium hydride (970 mg, 24.24 mmol) was added in batches; the reaction was stirred for 30 min with the temperature maintained, and then iodomethane (1.5 mL, 24.24 mmol) was added dropwise slowly. After addition, the reaction solution was moved to room temperature and stirred for 16 h. After completion of the reaction, the reaction was quenched by slow dropwise addition of water under cooling of an ice-water bath. The reaction solution was extracted with ethyl acetate, washed with saturated brine, and partitioned; the organic phases were combined, dried over anhydrous sodium sulfate, filtered, and the filtrate was concentrated under reduced pressure and purified to give 1.43 g of compound C13-2. Ms [M+H]+ 212

### 2. Synthesis of Compound C13-3

Compound C13-2 (1.0 g, 4.7 mmol) was dissolved in 25 mL of 1,4-dioxane, and bis(pinacolato)diboron (1.44 g, 5.66 mmol), 1,1′-bis(diphenylphosphino)ferrocene-palladium(II)dichloride dichloromethane complex (192 mg, 0.24 mmol), and potassium acetate (1.4 g, 14.1 mmol) were sequentially added; then the reaction solution was heated to 95° C. and stirred for 20 h under nitrogen protection. After completion of the reaction, the reaction solution was cooled to room temperature, added with saturated saline solution, and extracted with ethyl acetate; the organic phases were combined, dried with anhydrous sodium sulfate, filtered, and the filtrate was concentrated under reduced pressure and purified to give 600 mg of compound C13-3. Ms [M+H]+ 260.2

### 3. Synthesis of Compound C13-4

Compound C13-3 (300 mg, 1.15 mmol) was dissolved in 12 mL of tetrahydrofuran, and 12 mL of 1 M diluted hydrochloric acid aqueous solution was added to the reaction solution. The reaction solution was then stirred for 3 h at room temperature. After completion of the reaction, the reaction solution was evaporated directly under reduced pressure, recrystallized with PE/EA, and filtered; the filter cake was rinsed with petroleum ether, and then dried to give 70 mg of compound C13-4. Ms [M+H]+ 178.1

### 4. Synthesis of Compound C13-5

To a solution of Compound B (80 mg, 0.15 mmol) in 5 mL of N,N-dimethylformamide was added sequentially compound C13-4 (60 mg, 0.30 mmol), copper acetate (27 mg, 0.15 mmol) and pyridine (24 mg, 0.30 mmol) and then stirred overnight at 25° C. under oxygen atmosphere. After completion of the reaction, the reaction solution was diluted with ethyl acetate, washed with saturated saline, and partitioned. The organic phases were combined, dried with anhydrous sodium sulfate, and filtered; the filtrate was concentrated under reduced pressure and purified to give 70 mg of compound C13-5. Ms [M+H]+ 658.4

### 5. Synthesis of Compound C13

Compound C13-5 (70 mg, 0.11 mmol) was dissolved in 3 mL of methanol, and then 4 mL of 4M HCl methanol solution was added; the reaction solution was stirred at 25° C. for 30 min under nitrogen protection, and then heated up to 55° C. for 5 h. After completion of the reaction, the reaction solution was cooled to room temperature, concentrated under reduced pressure and purified to give 7 mg of compound TYK-00745 trifluoroacetate. Ms [M+H]+450.3.

NMR data of Compound C13: 1H NMR (400 MHz, Methanol-d4) δ8.24 (s, 1H), 7.23(t, J=7.9 Hz, 1H), 7.07(d, J=8.0 Hz, 1H), 6.62(d, J=7.8 Hz, 1H), 4.83(s, 2H), 4.36-4.28 (m, 1H), 3.97(d, J=9.1 Hz, 1H), 3.87(d, J=9.2 Hz, 1H), 3.60-3.50 (m, 2H), 3.49-3.47(m, 1H), 3.38-3.33(m, 2H), 3.10-2.94(m, 4H), 2.82(s, 3H), 2.09-1.99(m, 2H), 1.98-1.92(m, 1H), 1.81-1.74(m, 1H), 1.33(d, J=6.5 Hz, 3H).

### EXAMPLE 13

The compound synthesized in the present invention:

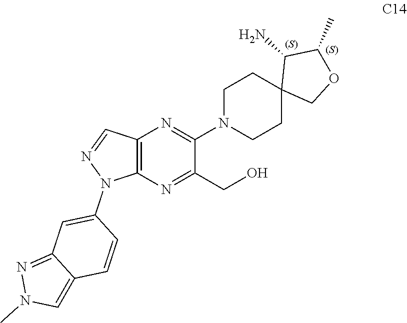

The synthetic route of compound C14 is as follows:

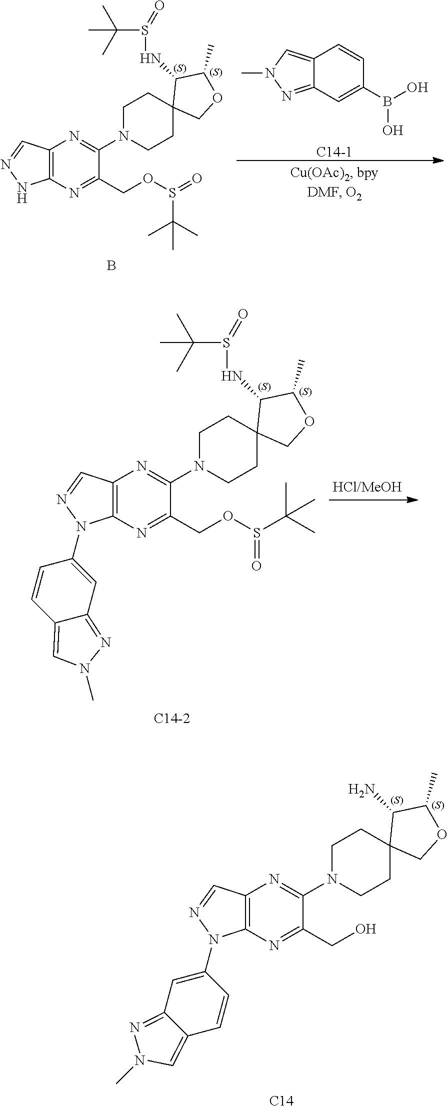

### 1. Synthesis of Compound C14-2

To a solution of Compound B (70 mg, 0.13 mmol) was dissolved in 5 mL of N,N-dimethylformamide was added sequentially compound C14-1 (46 mg, 0.26 mmol), copper acetate (24 mg, 0.13 mmol) and 2,2′-bipyridine (42 mg, 0.26 mmol) and then stirred at 25° C. for 2 hrs under oxygen atmosphere. After completion of the reaction, the reaction was diluted with ethyl acetate, and washed with saturated salt water. The organic phase was separated, dried with anhydrous sodium sulfate, and filtered; the filtrate was concentrated under reduced pressure and purified to give 74 mg of compound 14-2. Ms [M+H]+ 657.4

### 2. Synthesis of Compound C14

Compound C14-2 (74 mg, 0.11 mmol) was dissolved in 5 mL of methanol, and then 5 mL of 4M HCl methanol solution was added; the reaction solution was stirred at 25° C. for 30 min under nitrogen protection, and then heated up to 55° C. for 6 hours. After completion of the reaction, the reaction was cooled to room temperature, concentrated under reduced pressure and purified to give 23 mg of compound C14 trifluoroacetate. Ms [M+H]+ 449.2

NMR data of Compound C14: 1H NMR (400 MHz, Methanol-d4) δ8.55(s, 1H), 8.27-8.26(m, 2H), 8.06(dd, J=9.1, 1.8 Hz, 1H), 7.86(d, J=9.1 Hz, 1H), 4.90(s, 2H), 4.36-4.28(m, 1H), 4.25(s, 3H), 3.98(d, J=9.2 Hz, 1H), 3.87(d, J=9.1 Hz, 1H), 3.65-3.51(m, 2H), 3.49(d, J=4.1 Hz, 1H), 3.10-2.98(m, 2H), 2.11-2.00(m, 2H), 1.95(d, J=13.4 Hz, 1H), 1.78(d, J=12.8 Hz, 1H), 1.34(d, J=6.4 Hz, 3H).

### EXAMPLE 14

The compound synthesized in the present invention:

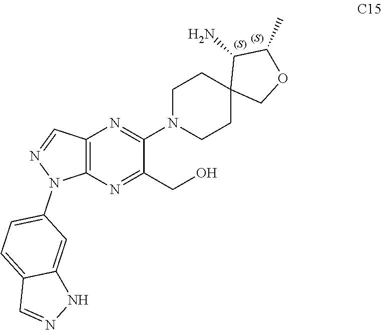

The synthetic route of compound C15 is as follows:

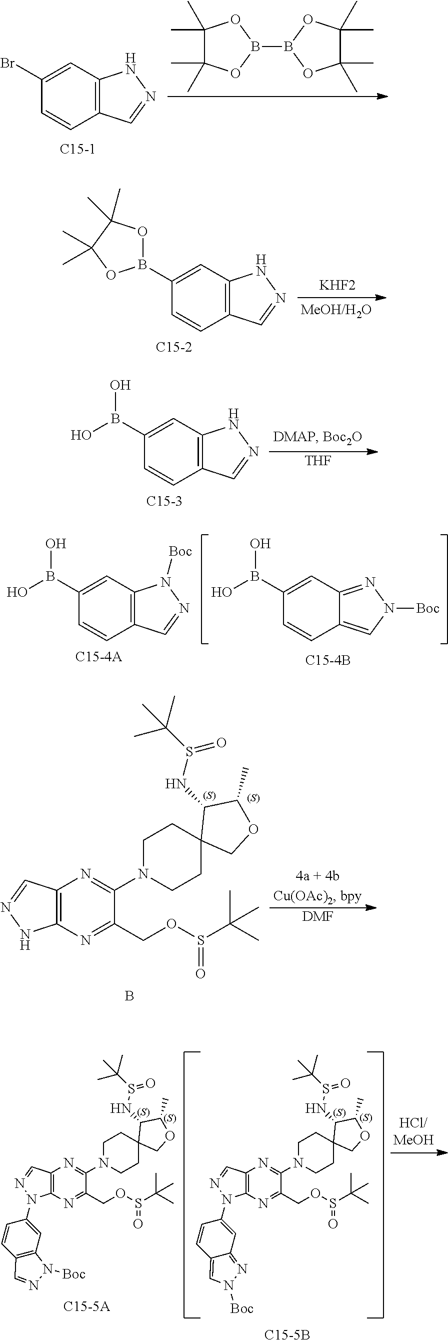

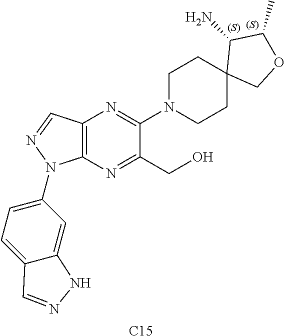

### 1. Synthesis of Compound C15-2

Compound C15-1 (2.0 g, 10.15 mmol) was dissolved in 50 mL of 1,4-dioxane, and bis(pinacolato)diboron (3.10 g, 12.18 mmol), 1,1′-bis(diphenylphosphino)ferrocene-palladium(II)dichloride dichloromethane complex (414 mg, 0.51 mmol), and anhydrous potassium acetate (3 g, 30.45 mmol) were sequentially added; then the reaction solution was heated to 95° C. and stirred for 20 h under nitrogen protection. After completion of the reaction, the reaction solution was cooled to room temperature, and diluted with saturated salt water, extracted with ethyl acetate and partitioned; the organic phases were combined, dried with anhydrous sodium sulfate, concentrated under reduced pressure and purified to give 3.0 g of compound C15-2. Ms [M+H]+ 245.1

### 2. Synthesis of Compound C15-3

To a solution of Compound C15-2 (1 g, 4.10 mmol) in 50 mL of methanol was added an aqueous solution (50 mL) of potassium fluorohydride (1.6 g, 20.5 mmol) and then stirred at room temperature for 1 hour. After completion of the reaction, the reaction solution was directly evaporated under reduced pressure; the residue was diluted and dissolved with acetonitrile, then extracted with petroleum ether to remove impurities, and partitioned; the acetonitrile phase was concentrated under reduced pressure and purified to give 400 mg of the compound C15-3. Ms [M+H]+ 163.0

### 3. Synthesis of Compound C15-4

To a solution of Compound C15-3 (400 mg, 2.47 mmol) in 15 mL of tetrahydrofuran was added sequentially 4-N,N-dimethylaminopyridine (30 mg, 0.25 mmol) and di-tert-butyl dicarbonate (1.1 g, 4.94 mmol). and stirred at room temperature for 3 hours. After completion of the reaction, the reaction was diluted with saturated salt water, extracted with ethyl acetate, and partitioned; the organic phases were combined, dried with anhydrous sodium sulfate, concentrated under reduced pressure and purified to give 80 mg of a mixture of compounds C15-4A and C15-4B. Ms [M+H]+ 263.1

### 4. Synthesis of Compound C15-5

To a solution of Compound B (80 mg, 0.15 mmol) in 5 mL of N,N-dimethylformamide was added sequentially a mixture of compound C15-4A and C15-4B (80 mg, 0.30 mmol), copper acetate (27 mg, 0.15 mmol) and 2′-bipyridine (50 mg, 0.30 mmol)and stirred at 25° C. for 20 hrs under oxygen atmosphere. After completion of the reaction, the reaction solution was diluted with ethyl acetate, and washed with saturated sodium chloride; the organic phases were combined, dried with anhydrous sodium sulfate, concentrated under reduced pressure and purified to give 90 mg of a mixture of compounds C15-5A and C15-5B. Ms [M+H]+ 743.4

### 5. Synthesis of Compound C15

The mixture of compounds C15-5A and C15-5B (90 mg, 0.12 mmol) was dissolved in 3 mL of methanol, and then 4 mL of 4M HCl methanol solution was added; the reaction solution was stirred at 25° C. for 30 min under nitrogen protection, and then warmed up to 55° C. for 5 hours. After completion of the reaction, the reaction solution was cooled to room temperature, concentrated under reduced pressure and purified to give compound C15 trifluoroacetate. Ms [M+H]+ 435.2.

NMR data of Compound C15: 1H NMR (400 MHz, Methanol-d4) δ8.50(s, 1H), 8.29(s, 1H), 8.19(dd, J=8.8, 1.8 Hz, 1H), 8.10(s, 1H), 7.92(d, J=8.8 Hz, 1H), 4.91(s, 2H), 4.36-4.27(m, 1H), 3.98(d, J=9.2 Hz, 1H), 3.88(d, J=9.2 Hz, 1H), 3.65-3.47(m, 3H), 3.10-2.97(m, 2H), 2.10-2.00(m, 2H), 1.99-1.91(m, 1H), 1.82-1.75(m, 1H), 1.34(d, J=6.5 Hz, 3H).

### EXAMPLE 15

The compound synthesized in the present invention:

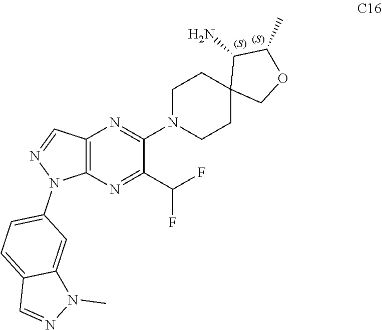

The synthetic route of compound C16is as follows:

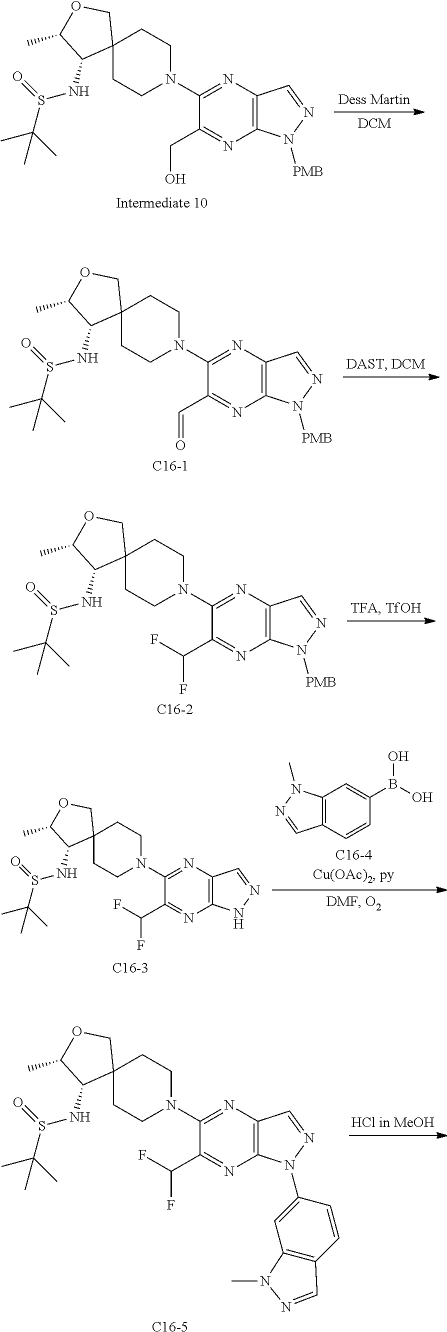

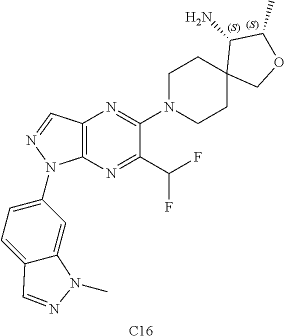

### 1. Synthesis of Compound C16-1

Intermediate 10 (600 mg, 1.1 mmol) was dissolved in 30 mL of dichloromethane, and Desmartin oxidizer (1.2 g, 2.76 mmol) was added; then the reaction solution was stirred at room temperature for 2 hours under nitrogen protection. After completion of the reaction, the reaction was quenched with saturated sodium bicarbonate and saturated sodium thiosulfate aqueous solution, and extracted with dichloromethane; the organic phases were combined, washed with saturated sodium bicarbonate aqueous solution, dried with anhydrous sodium sulfate, concentrated under reduced pressure and purified to give 500 mg of the compound C16-1. Ms [M+H]+ 541.3

NMR data of Compound C16-1:1H NMR(400 MHz, DMSO-d6) δ10.06(s, 1H), 8.28(s, 1H), 7.24(d, J=8.7 Hz, 1H), 6.87(d, J=8.7 Hz, 1H), 5.61(s, 2H), 5.14(d, J=10.7 Hz, 1H), 4.19-4.12(m, 1H), 3.82(d, =8.7 Hz, 1H), 3.7(s, 3H), 3.63-3.60(m, 1H), 3.55-3.5(m, 2H), 3.44(dd, J=10.8, 5.8 Hz, 1H), 3.13-3.00(m, 2H), 1.97-1.91(m, 2H), 1.70-1.61(m, 2H), 1.16(s, 9H), 1.10(d, J=6.3 Hz, 3H).

### 2. Synthesis of Compound C16-2

To a solution of Compound C16-1 (300 mg, 0.56 mmol) in 30 mL of dichloromethane was slowly added diethylamino sulfur trifluoride (267 mg, 1.67 mmol) dropwise under nitrogen protection and cooling of an ice bath. After addition, the reaction solution was moved to room temperature and stirred for 10 hours. After completion of the reaction, the reaction was quenched with saturated sodium bicarbonate aqueous solution, and extracted with dichloromethane; the organic phases were combined, dried with anhydrous sodium sulfate, concentrated under reduced pressure and purified to give 189 mg of compound C16-3. Ms [M+H]+ 563.3

3. Synthesis of compound C16-3

Compound C16-2 (180 mg, 0.32 mmol) was dissolved in 2 mL of trifluoroacetic acid, and the reaction solution was added with trifluoromethanesulfonic acid (0.2 mL)under cooling of an ice bath, and stirred under nitrogen protection for 5 min, then moved to room temperature, and stirred for another 2 hours. After the reaction, the reaction solution was poured into ice water, then quenched with saturated sodium bicarbonate aqueous solution, and extracted with ethyl acetate; the organic phases were combined, dried with anhydrous sodium sulfate, concentrated under reduced pressure and purified to give 141 mg of compound C16-3. Ms [M+H]+ 443.2

### 4. Synthesis of Compound C16-5

To a solution of Compound C16-3 (107 mg, 0.24 mmol) in 5 mL of N,N-dimethylformamide was added sequentially compound C16-4 (85 mg, 0.48 mmol), copper acetate (44 mg, 0.24 mmol) and pyridine (33 mg, 0.48 mmol) and stirred at 25° C. for 16 hrs under oxygen atmosphere. After completion of the reaction, the reaction solution was diluted with ethyl acetate, and washed with saturated sodium chloride solution; the organic phase was dried with anhydrous sodium sulfate, concentrated under reduced pressure and purified to give 159 mg of compound C16-5. Ms [M+H]+ 573.3

### 5. Synthesis of Compound C16

Compound C16-5 (150 mg, 0.27 mmol) was dissolved in 5 mL of methanol, and then 5 mL of 4M HCl methanol solution was added; the reaction solution was stirred at 25° C. for 30 min under nitrogen protection, and then heated up to 55° C. for 6 hours. After completion of the reaction, the reaction was cooled to room temperature, concentrated under reduced pressure and purified to give 159 mg of compound C16 trifluoroacetate. Ms [M+H]+ 469.3

NMR data of Compound C16: 1H NMR (400 MHz, Methanol-d4) δ8.47(s, 1H), 8.43(s, 1H), 8.19(dd, J=8.8, 1.7 Hz, 1H), 8.07(s, 1H), 7.93(d, J=8.8 Hz, 1H), 7.17(t, J=53.7 Hz, 1H), 4.35-4.29(m, 1H), 4.13(s, 3H), 3.99(d, J=9.2 Hz, 1H), 3.89(d, J=9.2 Hz, 1H), 3.61-3.41(m, 3H), 3.20-3.04(m, 2H), 2.16-2.02(m, 2H), 1.97(d, J=13.4 Hz, 1H), 1.80(d, J=12.9 Hz, 1H), 1.34(d, J=6.5 Hz, 3H).

### EXAMPLE 16

The compound synthesized in the present invention:

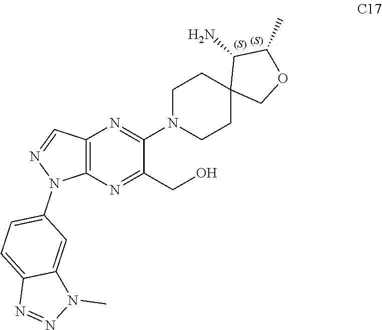

The synthetic route of compound C17 is as follows:

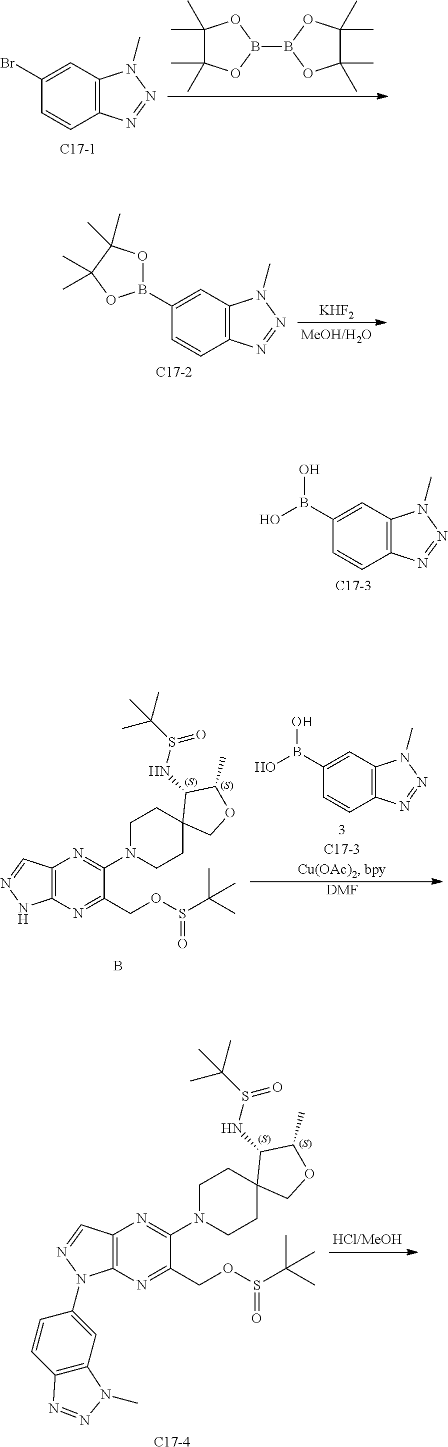

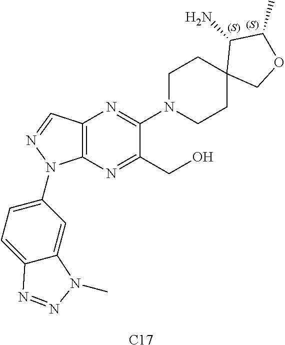

### 1. Synthesis of Compound C17-2

Compound C17-1 (1.0 g, 4.7 mmol) was dissolved in 25 mL of 1,4-dioxane, and bis(pinacolato)diboron (1.8 g, 7.07 mmol), 1,1′-bis(diphenylphosphino)ferrocene-palladium(II)dichloride dichloromethane complex (200 mg, 0.24 mmol), and potassium acetate (1.4 g, 14.1 mmol) were sequentially added; then the reaction solution was heated to 95° C. and stirred for 20 h under nitrogen protection. After the reaction, the reaction solution was cooled to room temperature, diluted with saturated salt water, extracted with ethyl acetate, and partitioned; the organic phases were combined, dried with anhydrous sodium sulfate, concentrated under reduced pressure and purified to give 1.6 g of compound C17-2. Ms [M+H]+ 260.1

### 2. Synthesis of Compound C17-3

Compound C17-2 (300 mg, 1.23 mmol) was dissolved in 20 mL of methanol, and an aqueous solution (20 mL) of potassium fluorohydride (480 mg, 6.15 mmol) was added; then the reaction solution was stirred at room temperature for 1 hour. After completion of the reaction, the reaction solution was directly evaporated under reduced pressure, and the residue was diluted and dissolved with acetonitrile, then extracted with petroleum ether to remove impurities, and partitioned; the acetonitrile phase was concentrated under reduced pressure and purified to give 90 mg of the compound C17-3. Ms [M+H]+ 178.0

NMR data of Compound C17-3: 1H NMR (400 MHz, DMSO-d6) δ8.30(s, 2H), 8.09-8.00(m, 1H), 7.98-7.93(m, 1H), 7.83-7.77(m, 1H), 4.32(s, 3H).

### 3. Synthesis of Compound C17-4

To a solution of Compound B (80 mg, 0.15 mmol) in 5 mL of N,N-dimethylformamide was added sequentially compound C17-3 (60 mg, 0.30 mmol), copper acetate (27 mg, 0.15 mmol) and 2,2′-bipyridine (24 mg, 0.30 mmol) and stirred overnight at 25° C. under oxygen atmosphere. After completion of the reaction, the reaction solution was diluted with ethyl acetate, washed with saturated sodium chloride solution, dried with anhydrous sodium sulfate, concentrated under reduced pressure and purified to give 90 mg of compound C17-4. Ms [M+H]+ 658.3

NMR data of Compound C17-4: 1H NMR (400 MHz, DMSO-d6) δ8.69-8.65(m, 1H), 8.63(s, 1H), 8.44-8.38(m, 1H), 8.24-8.20(m, 1H), 5.40(s, 2H), 5.16(d, J=10.7 Hz, 1H), 4.38(s, 3H), 4.21-4.13(m, 1H), 3.87-3.82(m, 1H), 3.57-3.52(m, 1H), 3.51-3.44(m, 1H), 3.43-3.38(m, 2H), 3.04-2.90(m, 2H), 2.04-1.95(m, 2H), 1.76-1.64(m, 2H), 1.18(s, 9H), 1.15(s, 9H), 1.12(d, J=6.4 Hz, 3H).

### 4. Synthesis of Compound C17

Compound C17-4 (90 mg, 0.14 mmol) was dissolved in 3 mL of methanol, and 4 mL of HCl methanol (4M) was added; the reaction solution was stirred at 25° C. for 30 min under the protection of nitrogen, and then heated to 55° C. and stirred for 5 h. After completion of the reaction, the reaction solution was cooled to room temperature, concentrated under reduced pressure and purified to give C17 trifluoroacetate. Ms [M+H]+ 450.2

NMR data of Compound C17: 1H NMR (400 MHz, Methanol-d4) 8 8.76(s, 1H), 8.55(dd, J=9.1, 1.9 Hz, 1H), 8.33(s, 1H), 8.13(d, J=9.1 Hz, 1H), 4.94(s, 2H), 4.42(s, 3H), 4.36-4.29(m, 1H), 3.98(d, J=9.2 Hz, 1H), 3.88(d, J=9.1 Hz, 1H), 3.62-3.51(m, 2H), 3.51-3.47(m, 1H), 3.10-2.98(m, 2H), 2.10-2.01(m, 2H), 2.00-1.93(m, 1H), 1.83-1.75(m, 1H), 1.34(d, J=6.5 Hz, 3H).

### Example 17

The compound synthesized in the present invention:

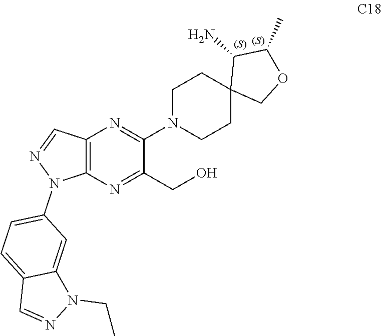

The synthetic route of compound C18 is as follows:

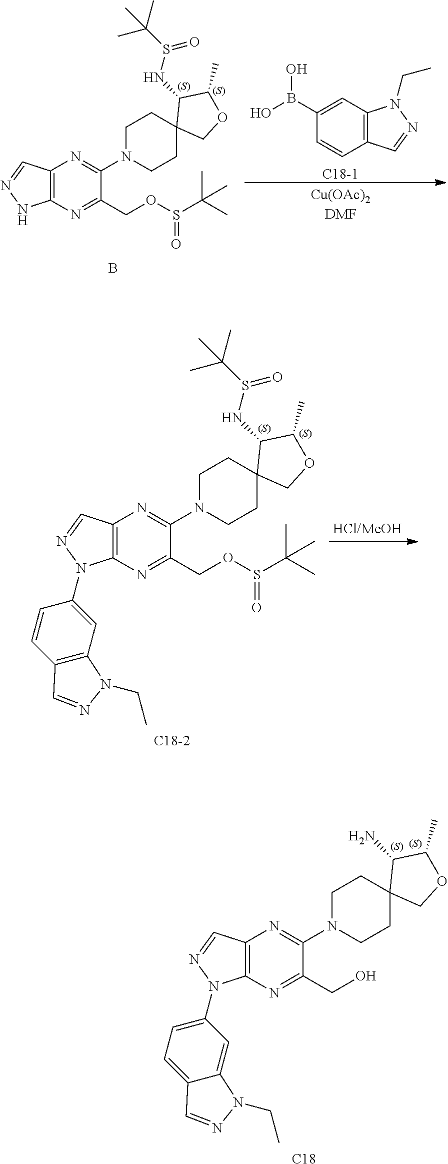

### 1. Synthesis of Compound C18-2

To a solution of Compound B (80 mg, 0.15 mmol) was dissolved in 5 mL of N,N-dimethylformamide was added sequentially compound C18-1 (60 mg, 0.30 mmol), copper acetate (27 mg, 0.15 mmol) and 2,2′-bipyridine (47 mg, 0.30 mmol) and stirred overnight at 25° C. under oxygen atmosphere. After the reaction, the reaction solution was diluted with ethyl acetate, washed with saturated sodium chloride, dried with anhydrous sodium sulfate, and filtered; the filtrate was concentrated under reduced pressure and purified to yield 100 mg of compound C18-2. Ms [M+H]+ 671.4

### 2. Synthesis of Compound C18

Compound C18-2 (100 mg, 0.15 mmol) was dissolved in 3 mL of methanol, and 4 mL of 4M HCl methanol solution was added; the reaction solution was stirred under nitrogen protection at 25° C. for 30 min, and then heated to 55° C. and stirred for 5 h. After completion of the reaction, the reaction solution was cooled to room temperature, concentrated under reduced pressure and purified to give C18 trifluoroacetate. Ms [M+H]+ 463.2.

NMR data of Compound C18: 1H NMR (400 MHz, Methanol-d4) δ8.53(s, 1H), 8.30(s, 1H), 8.21-8.16(m, 1H), 8.07(s, 1H), 7.91(d, J=8.8 Hz, 1H), 4.93(s, 2H), 4.55(q, J=7.3 Hz, 2H), 4.36-4.28(m, 1H), 3.98(d, J=9.1 Hz, 1H), 3.88(d, J=9.1 Hz, 1H), 3.62-3.47(m, 3H), 3.11-2.98(m, 2H), 2.12-2.01(m, 2H), 2.00-1.92(m, 1H), 1.83-1.75 (m, 1H), 1.53(t, J=7.2 Hz, 3H), 1.34(d, J=6.5 Hz, 3H).

### EXAMPLE 18

The compound synthesized in the present invention:

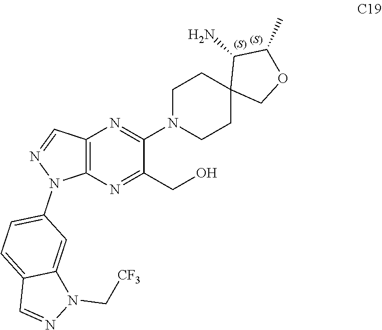

The synthetic route of compound C19 is as follows:

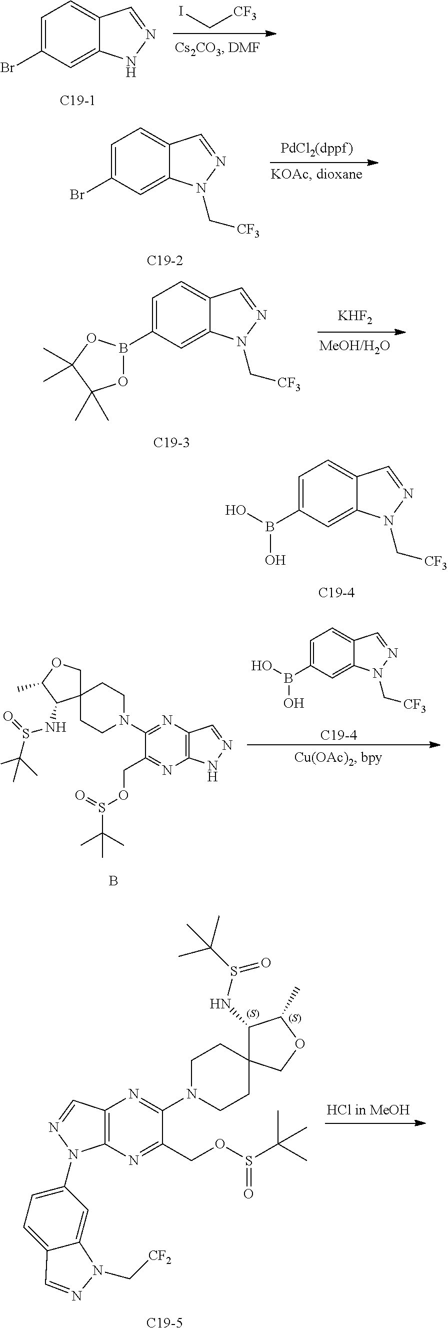

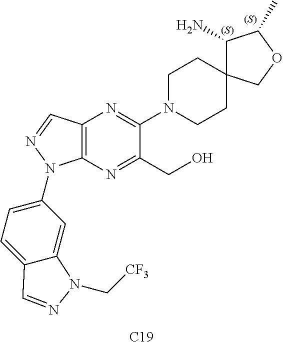

### 1. Synthesis of Compound C19-2

To a solution of Compound C19-1 (1.0 g, 5.08 mmol) in 10 mL of N,N-dimethylformamide was added cesium carbonate (5.0 g, 15.24 mmol), and stirred at room temperature for 0.5 h. The reaction solution was added with trifluoroiodoethane (2.13 g, 10.2 mmol) and heated to 50° C. and stirred for Sh. After the reaction, the reaction solution was cooled to room temperature, quenched with water, and extracted with ethyl acetate; the organic phases were combined, dried with anhydrous sodium sulfate, concentrated under reduced pressure and purified to give 552 mg of compound C19-2. Ms [M+H]+ 278.9

NMR data of Compound C19-2: 1H NMR (90 MHz, CDCl3) δ8.04(s, 1H), 7.67-7.57(m, 2H), 7.38-7.2(m, 1H), 4.90(q, 3H).

### 2. Synthesis of Compound C19-3

Compound C19-2 (500 mg, 1.8 mmol) was dissolved in 30 mL of 1,4-dioxane, and bis(pinacolato)diboron (914 mg, 3.6 mmol), 1,l′-bis(diphenylphosphino)ferrocene-palladium(II)chloride (73 mg, 0.09 mmol), and potassium acetate (529 mg, 5.4 mmol) were sequentially added; then the reaction solution was stirred at 95° C. for 20 h under nitrogen protection. After completion of the reaction, the reaction solution was cooled to room temperature, diluted with water, and extracted with ethyl acetate; the organic phases were combined, dried with anhydrous sodium sulfate, concentrated under reduced pressure and purified to give 930 mg of compound C19-3. Ms [M+H]+ 327.1

### 3. Synthesis of Compound C19-4

To a solution of Compound C19-3 (400 mg, 1.22 mmol) in 40 mL of methanol was added an aqueous solution (40 mL) of potassium fluorohydride (478 mg, 6.13 mmol), and then stirred at room temperature for 1 hour. After completion of the reaction, the reaction solution was directly evaporated under reduced pressure, and the residue was diluted and dissolved with acetonitrile, then extracted with petroleum ether to remove impurities, and partitioned; the acetonitrile phase was concentrated under reduced pressure to give 80 mg of compound C19-4. Ms [M+H]+ 245.0

### 4. Synthesis of Compound C19

Referring to the synthesis of compound C18, the data are shown in Table I.

### EXAMPLE 19

The compound synthesized in the present invention:

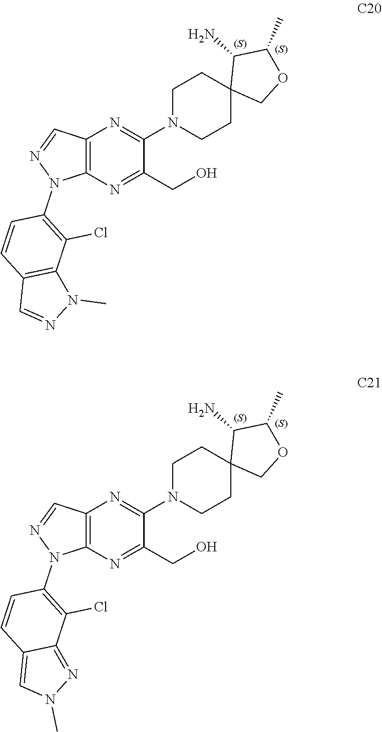

The synthetic route of compound C20 and C21 are as follows:

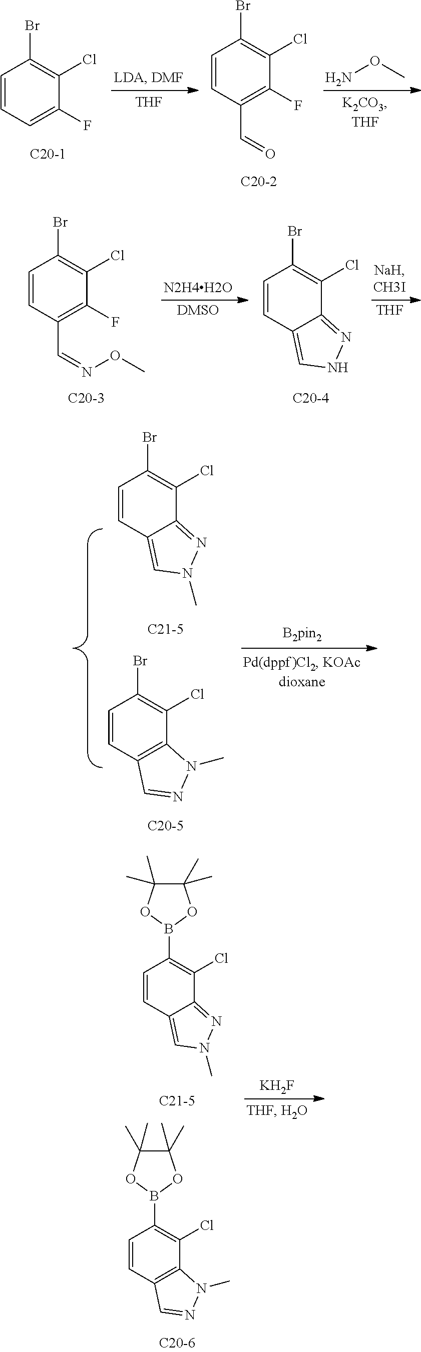

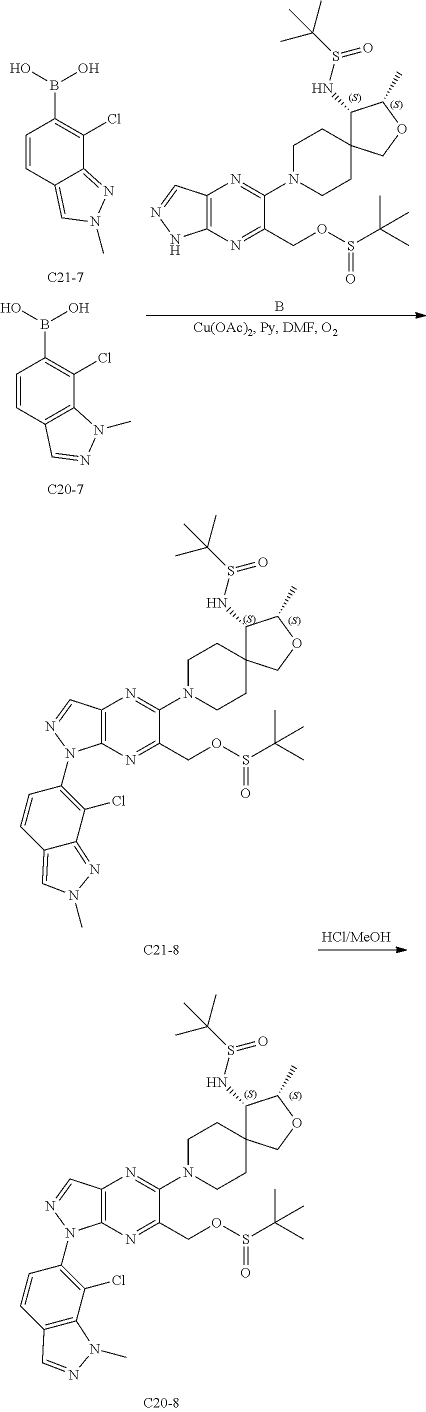

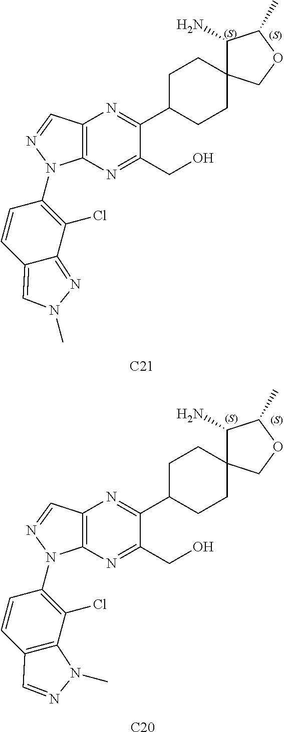

### 1. Synthesis of Compound C20-2

Compound C20-1 (5 g, 23.87 mmol) was dissolved in 25 mL of tetrahydrofuran, then the reaction was cooled down to −62° C. under dry ice bath, added slowly with LDA (26.3 mL, 26.26 mmol) and stirred at −62° C. for 1 h; then DMF (2.617 g, 35.81 mmol) was added dropwise slowly. The reaction solution was naturally warmed to room temperature and stirred for 3 h under nitrogen protection. After completion of the reaction, the reaction solution was placed in an ice-water bath, quenched by slowly adding saturated ammonium chloride aqueous solution, and extracted with ethyl acetate; the organic phases were combined, dried with anhydrous sodium sulfate, and concentrated under reduced pressure and purified to give 2.559 g compound C20-2. Ms [M+H]+ 236.9

### 2. Synthesis of Compound C20-3

To a solution of compound C20-2 (2.459 g, 10.36 mmol) in 25 mL of tetrahydrofuran was added potassium carbonate (1.714 g, 12 43 mmol) and methoxyammonia (951 mg, 11 39 mmol) and stirred at 45° C. for 12 hours under nitrogen protection. After the reaction, the reaction solution was cooled to room temperature, quenched with water, and extracted with ethyl acetate; the organic phases were combined, dried with anhydrous sodium sulfate, concentrated under reduced pressure and purified to give 2.250 g of compound C20-3. Ms [M+H]+265.9

### 3. Synthesis of Compound C20-4

Compound C20-3 (1.45 g, 5.44 mmol) was dissolved in 15 mL of dimethyl sulfoxide, and then hydrazine hydrate (544 mg, 10.88 mmol) was added; the reaction solution was stirred at 100° C. for 12 hours under nitrogen protection. After completion of the reaction, the reaction solution was cooled to room temperature, quenched with water, and extracted with ethyl acetate; the organic phases were combined, dried with anhydrous sodium sulfate, concentrated under reduced pressure and purified to give 1.22 g of compound C20-4. Ms [M+H]+ 230.9

### 4. Synthesis of Compounds C20-5 and C21-5

A solution of Compound C20-4 (1.220 g, 5.27 mmol) in 15 mL of tetrahydrofuran was placed in an ice-water bath, added slowly with sodium hydride (253 mg, 6.32 mmol) and stirred at 0° C. for 60 min. Then iodomethane (1.123 g, 7.91 mmol) was added slowly and dropwise, and then the reaction solution was warmed to room temperature and stirred overnight under nitrogen protection. After completion of the reaction, the reaction solution was placed in an ice-water bath, and quenched by slowly adding water, and extracted with ethyl acetate; the organic phases were combined, dried with anhydrous sodium sulfate, concentrated under reduced pressure and purified to give compounds C20-5 and C21-5.

### 5. Synthesis of Compounds C20-7 and C21-7

Compounds C20-7 and C21-7 was synthesized by referring to the synthesis of compound C19-4.

### 6. Synthesis of Compounds C20 and C21

Compounds C20 and C21was synthesized by referring to the synthesis of compound C18, and the data are shown in Table I.

### EXAMPLE 20

The compound synthesized in the present invention:

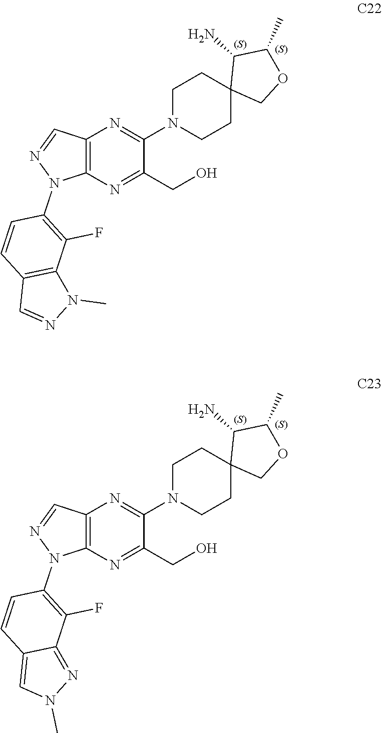

The synthetic route of compounds C22 and C23 are as follows:

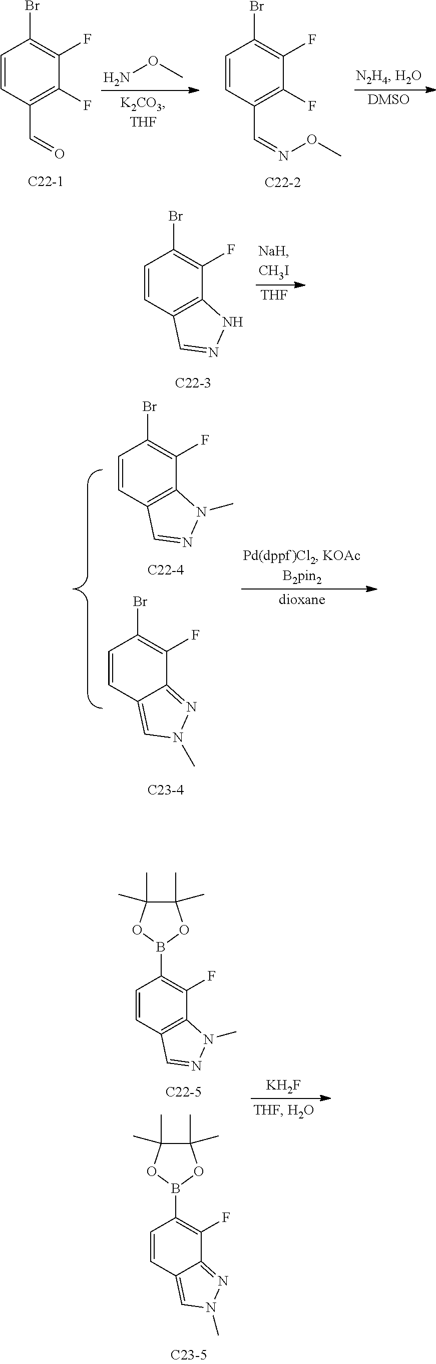

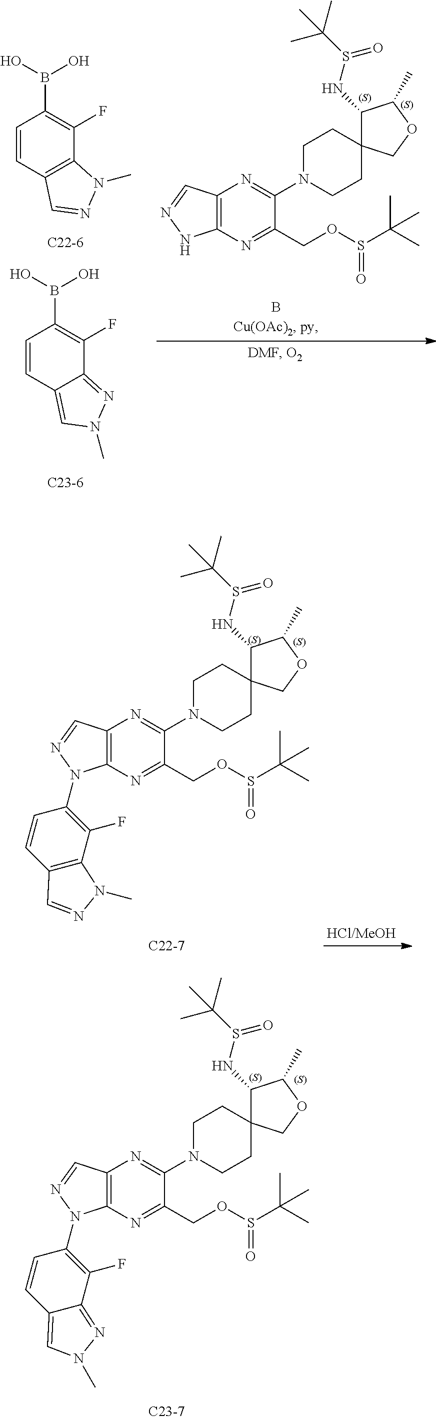

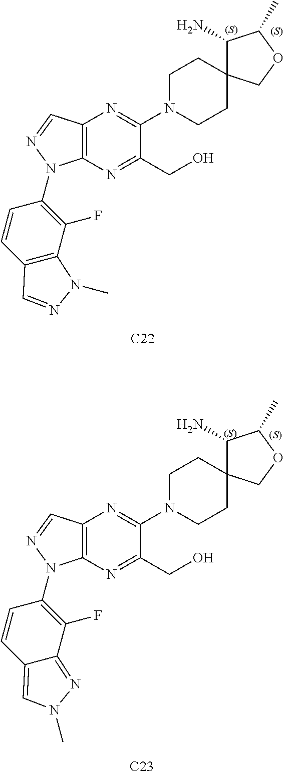

### 1. Synthesis of Compound C22-2

Compound C22-1 (3.0 g, 13.5 mmol) was dissolved in 60 mL of tetrahydrofuran, and then potassium carbonate (2.2 g, 16.3 mmol), methoxy ammonia hydrochloride (1.3 g, 14.9 mmol) were added sequentially; the reaction solution was stirred at 45° C. for 3 h under nitrogen protection. After completion of the reaction, the reaction solution was cooled to room temperature and then filtered through diatomaceous earth; the filter cake was washed with ethyl acetate, and the filtrate was concentrated under reduced pressure and purified to give 3.0 g of compound C22-2. Ms [M+H]+ 250.0

### 2. Synthesis of Compound C22-3

Compound C22-2 (3.0 g, 12.1 mmol) was dissolved in 15 mL of dimethyl sulfoxide, then 7 mL of hydrazine hydrate was added, and the reaction solution was stirred at 90° C. for 36 hours under nitrogen protection. After completion of the reaction, the reaction solution was cooled to room temperature, quenched with water, and extracted with ethyl acetate; the organic phases were combined, dried with anhydrous sodium sulfate, filtered, and the filtrate was concentrated under reduced pressure and purified to give 2.2 g of compound C22-3. Ms [M+H]+ 215.0

### 3. Synthesis of compounds C22-4 and C23-4

Compound C22-3 (2.2 g, 10.1 mmol) was dissolved in 40 mL of tetrahydrofuran, and under cooling of ice-water bath, sodium hydride (489 mg, 12.2 mmol) with a content of 60% was added in batches. After addition, the reaction solution was slowly warmed up to room temperature and stirred for 1 h, then cooled down to 0° C., and slowly added with methylene iodide (2.2 g, 15.3 mmol) dropwise. After the addition, the reaction solution was warmed to room temperature and stirred. After completion of the reaction, the reaction solution was placed in an ice-water bath, quenched by slowly adding water, and extracted with ethyl acetate; the organic phases were combined, dried with anhydrous sodium sulfate, filtered, and the filtrate was concentrated under reduced pressure and purified to give 710 mg of Compound C22-4 and 1.44 g of Compound C23-4. Ms [M+H]+ 229.0

### 4. Synthesis of Compound C22-5 and C23-5

Compound C22-4 (900 mg, 3.95 mmol) was dissolved in 10 mL of 1,4-dioxane, and bis(pinacolato)diboron (1.2 g, 4.74 mmol), 1,1′-bis(diphenylphosphino)ferrocene-palladium(II) dichloride dichloromethane complex (144 mg, 0.2 mmol), and potassium acetate (1.16 g, 11.85 mmol) were sequentially added;

then the reaction solution was stirred at 95° C. for 20 h under nitrogen protection. After completion of the reaction, the reaction solution was cooled to room temperature, quenched with water, and extracted with ethyl acetate; the organic phases were combined, dried with anhydrous sodium sulfate, concentrated under reduced pressure and purified to give 996 mg of compound C22-5. Ms [M+H]+ 277.1

Compound C23-4 (1.84 g, 8.07 mmol) was dissolved in 20 mL of 1,4-dioxane, and bis(pinacolato)diboron (2.46 g, 9.68 mmol), 1,l′-bis(diphenylphosphino)ferrocene-palladium(II) chloride (295 mg, 0.4 mmol), and potassium acetate (2.37 g, 24.21 mmol) were sequentially added; then the reaction solution was stirred at 95° C. for 12 h under nitrogen protection. After completion of the reaction, the reaction solution was cooled to room temperature, quenched with water, and extracted with ethyl acetate; the organic phases were combined, dried with anhydrous sodium sulfate, concentrated under reduced pressure and purified to give 1.98 g of compound C23-5. Ms [M+H]+ 277.1

### 5. Synthesis of Compound C22-6 and C23-6

To a solution of Compound C22-5 (996 mg, 3.6 mmol) in 100 mL of methanol was added an aqueous solution (100 mL) of potassium fluorohydride (254 mg, 3.44 mmol) and then stirred at room temperature for 30 min. After completion of the reaction, the reaction solution was directly spun dry, dissolved with acetonitrile, and extracted with petroleum ether; then the acetonitrile phase was retained, and filtered to remove the residue, and the filtrate was concentrated under reduced pressure to give 392 mg of compound C22-6. Ms [M+H]+ 195.0

To a solution of Compound C23-5 (500 mg, 1.8 mmol) in 50 mL of methanol was added an aqueous solution (50 mL) of potassium fluorohydride (700 mg, 9 mmol) and then stirred at room temperature for 30 min. After completion of the reaction, the reaction solution was directly spun dry, dissolved with acetonitrile, and extracted with petroleum ether; then the acetonitrile phase was retained, filtered to remove the residue, and concentrated under reduced pressure to give 102 mg of compound C23-6. Ms [M+H]+ 195.0

### 6. Synthesis of Compound C22 and C23

Compound C22 and C23 was synthesized by referring to the synthesis of compound C18, the data are shown in Table I.

### EXAMPLE 21

The compound synthesized in the present invention:

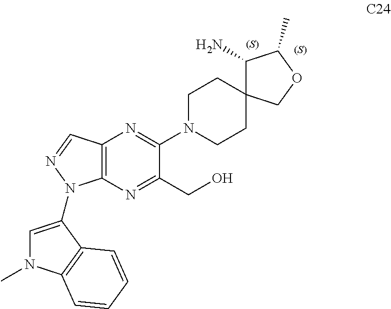

The synthetic route of compound C24 is as follows:

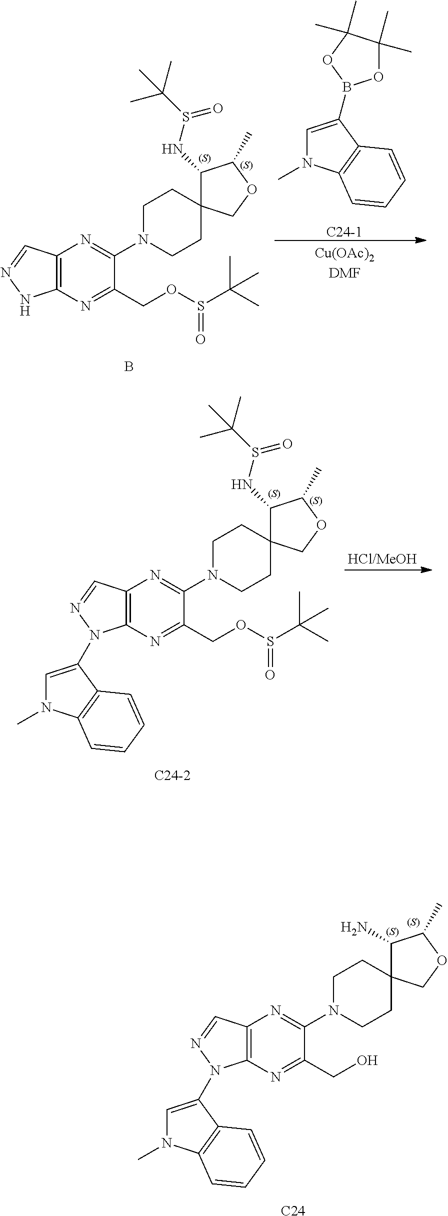

### 1. Synthesis of Compound C24-2

To a solution of Compound B (80 mg, 0.15 mmol) in 5 mL of N,N-dimethylformamide was added sequentially compound C14-1 (192 mg, 0.75 mmol), copper acetate (27 mg, 0.15 mmol) and 2,2′-bipyridine (47 mg, 0.20 mmol) and stirred at 90° C. for 7 hrs under oxygen atmosphere. After the reaction, the reaction solution was diluted with ethyl acetate, washed with saturated sodium chloride, dried with anhydrous sodium sulfate, and filtered; the filtrate was concentrated under reduced pressure and purified to give compound C24-2. Ms [M+H]+ 656.3

### 2. Synthesis of Compound C24

Compound C24 was synthesized by referring to the synthesis of compound C18 and the data are shown in Table I.

### EXAMPLE 22

The compound synthesized in the present invention:

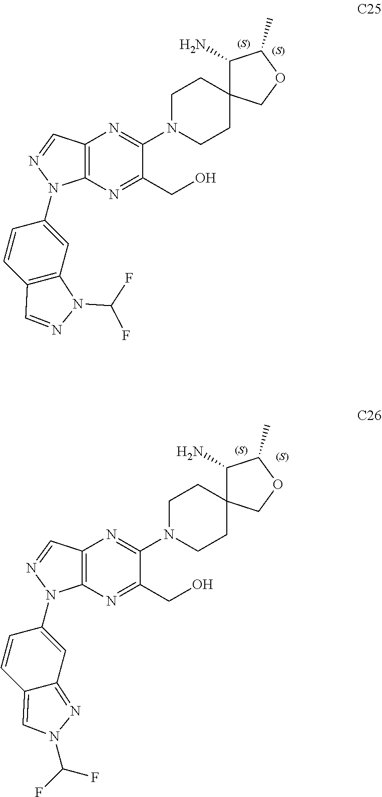

The synthetic route of compound C25 and C26 are as follows:

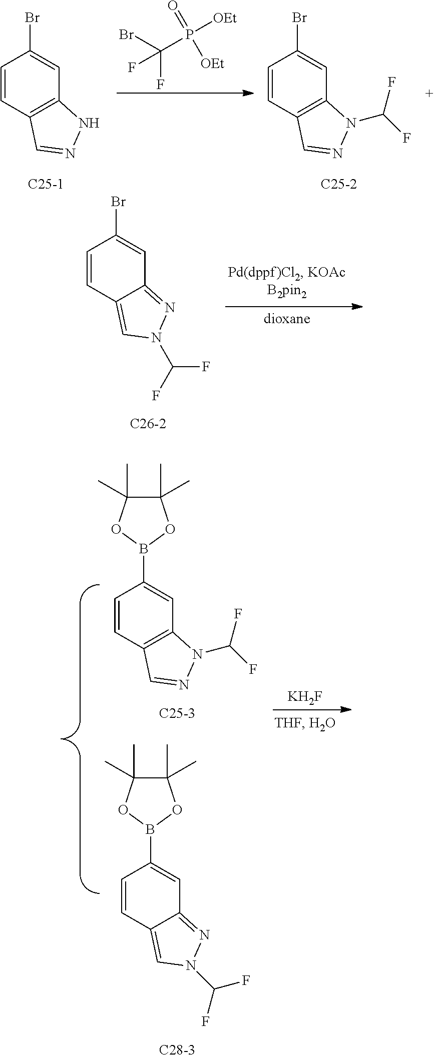

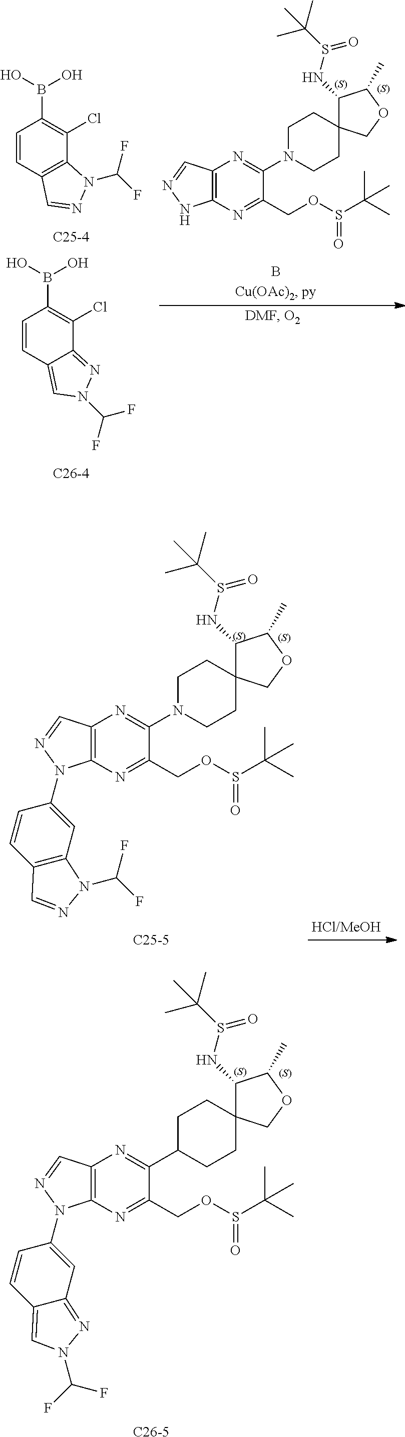

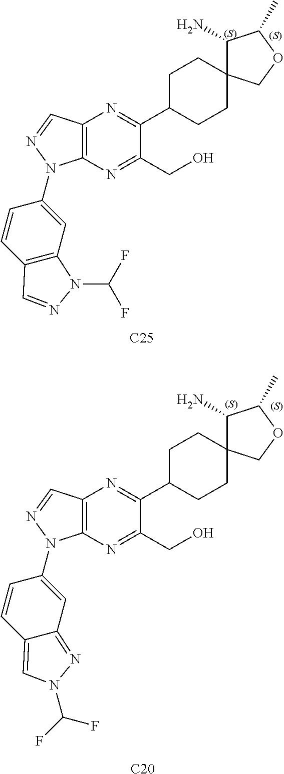

### 1. Synthesis of Compound C25-2 and C26-2

Compound C25-1 (5.0 g, 20.3 mmol) was dissolved in 200 mL of acetonitrile, and then potassium carbonate (10.75 g, 40.6 mmol), diethyl bromodifluoromethyl phosphate (8.15 g, 24.3 mmol) were added sequentially; the reaction solution was stirred for 12 hours at room temperature under nitrogen protection. After completion of the reaction, the reaction was quenched with water, extracted with ethyl acetate, washed with saturated salt water, dried with anhydrous sodium sulfate, concentrated under reduced pressure and purified to give 3.29 g of a mixture of Compound C25-2 and Compound C26-2. Ms [M+H]+ 246.9

### 2. Synthesis of Compounds C25-3 and C26-3

A mixture of Compounds C25-2 and C26-2(1.2 g, 4.8 mmol) was dissolved in 60 mL of 1,4-dioxane, and bis(pinacolato)diboron (2.48 g, 9.7 mmol), 1,1′-bis(diphenylphosphino)ferrocene-palladium(II) chloride (175 mg, 0.24 mmol), and potassium acetate (1.4 g, 14.4 mmol) were sequentially added; then the reaction solution was stirred at 95° C. for 12 h under nitrogen protection. After completion of the reaction, the reaction solution was cooled to room temperature, quenched with water, and extracted with ethyl acetate;

the organic phases were combined, dried with anhydrous sodium sulfate, concentrated under reduced pressure and purified to give 120 mg of compound C25-3 and 1.165 g of compound C26-3.

Compound C25-3: Ms [M+H]+ 295.1

Compound C26-3: Ms [M+H]+ 295.1

NMR data of Compound C26-3: 1H NMR (400 MHz, DMSO-d6) δ8.90(d, J=1.0 Hz, 1H), 8.17(t, J=60 Hz, 1H), 8.05(d, J=1.3 Hz, 1H), 7.78(dd, J=8.6, 1.1 Hz, 1H), 7.36(d, J=8.5 Hz, 1H), 1.32(s, 12H).

### 3. Synthesis of Compound C25 and C26

Compound C25 and compound C26 were synthesized with reference to the synthesis of compounds C22 and C23 and the data are shown in Table I.

### EXAMPLE 23

The compound synthesized in the present invention:

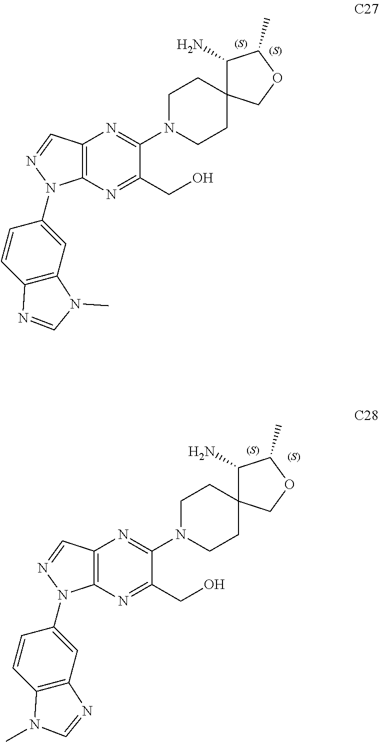

The synthetic route of compounds C27 and C28 are as follows:

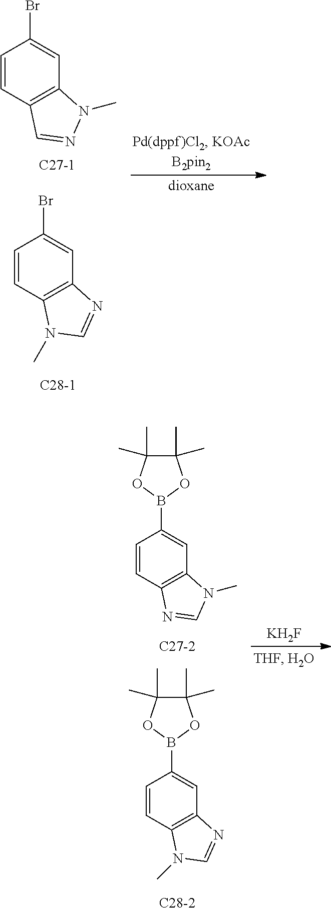

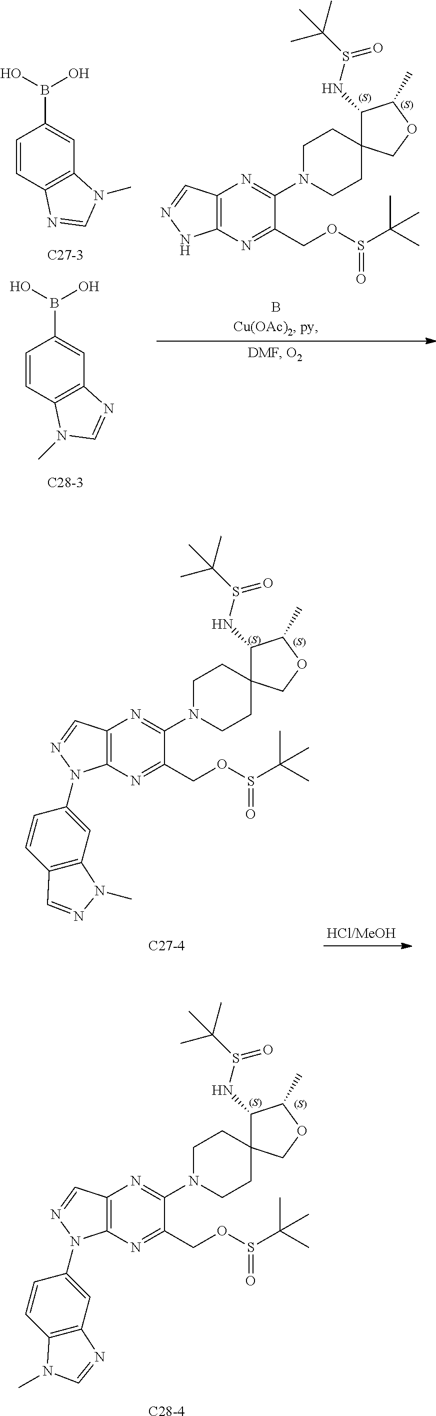

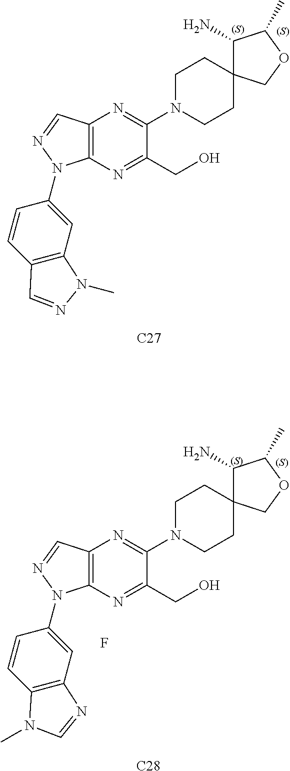

Compound C27 and compound C28 were synthesized with reference to the synthesis of compound C22, and the data are shown in Table I.

### EXAMPLE 24

The compound synthesized in the present invention:

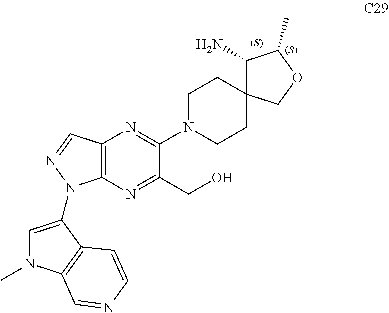

The synthetic route of compound C29 is as follows:

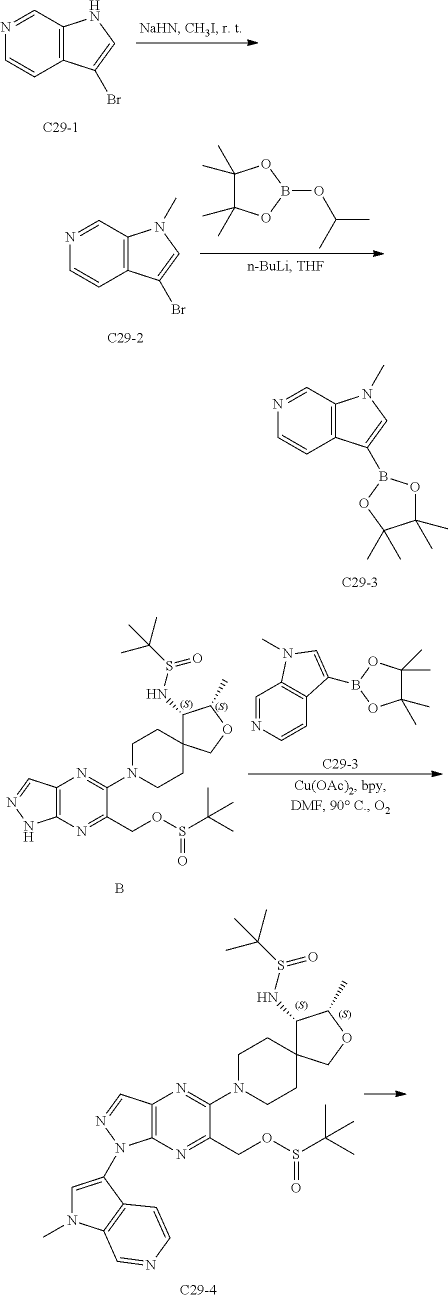

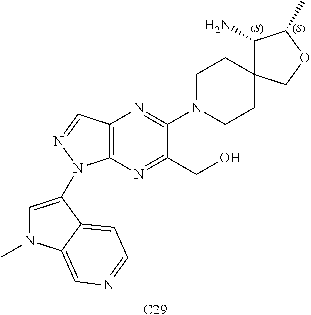

### 1. Synthesis of Compound C29-2

To a solution of Compound C29-1 (2.0 g, 10.15 mmol) in 20 mL of N,N-dimethylformamide was added 60% sodium hydride (609 mg, 15.23 mmol) under ice bath, and stirred under nitrogen protection for 0.5 h at room temperature; then methyl iodide (1.86 g, 13.2 mmol) was added dropwise under cooling of ice water bath. After addition, the reaction solution was moved to room temperature and stirred for 2 hours. After completion of the reaction, the reaction was quenched with water, and extracted with ethyl acetate; the organic phases were combined, dried with anhydrous sodium sulfate, concentrated under reduced pressure and purified to give 1.29 g of compound C29-2. Ms [M+H]+ 210.9

### 2. Synthesis of Compound C29-3

Compound C29-2 (700 mg, 3.33 mmol) was dissolved in 7 mL of tetrahydrofuran, and the reaction solution was cooled to −78° C. under nitrogen protection; then n-butyl lithium (2.5 M, 2 mL, 5 mmol) solution in tetrahydrofuran was added dropwise, and the temperature was maintained. The reaction solution was stirred for 0.5 h before dropwise addition of isopropoxyborate (930 mg, 5 mmol), then stirred at −78° C. for 1 h. After completion of the reaction, the reaction was quenched by dropwise addition of water at −78° C., then warmed up to room temperature, and extracted with ethyl acetate; the organic phases were combined, dried with anhydrous sodium sulfate, concentrated under reduced pressure and purified to give 170 mg of compound C29-3. Ms [M+H]+ 259.1

### 3. Synthesis of Compound C29-4

Compound B (80 mg, 0.155 mmol) was dissolved in 5 mL of N,N-dimethylformamide, and compound C29-3 (120 mg, 0.46 mmol), copper acetate (42 mg, 0.23 mmol) and pyridine (72 mg, 0.465 mmol) were added sequentially. The reaction solution was stirred at 95° C. for 16 hrs under oxygen atmosphere. After completion of the reaction, the reaction solution was diluted with ethyl acetate, and washed with saturated sodium chloride solution; the organic phase was separated, dried with anhydrous sodium sulfate, concentrated under reduced pressure and purified to afford compound C29-4. Ms [M+H]+ 657.3

### 4. Synthesis of Compound C29

Compound C29 was synthesized by referring to the synthesis of compound C22, and the data are shown in Table I.

### EXAMPLE 25

The compound synthesized in the present invention:

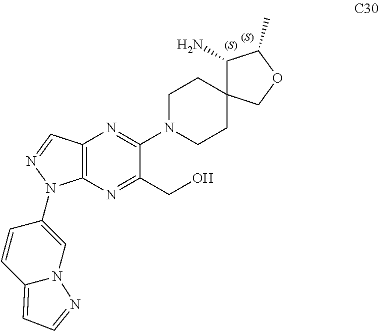

The synthetic route of compound C30 is as follows:

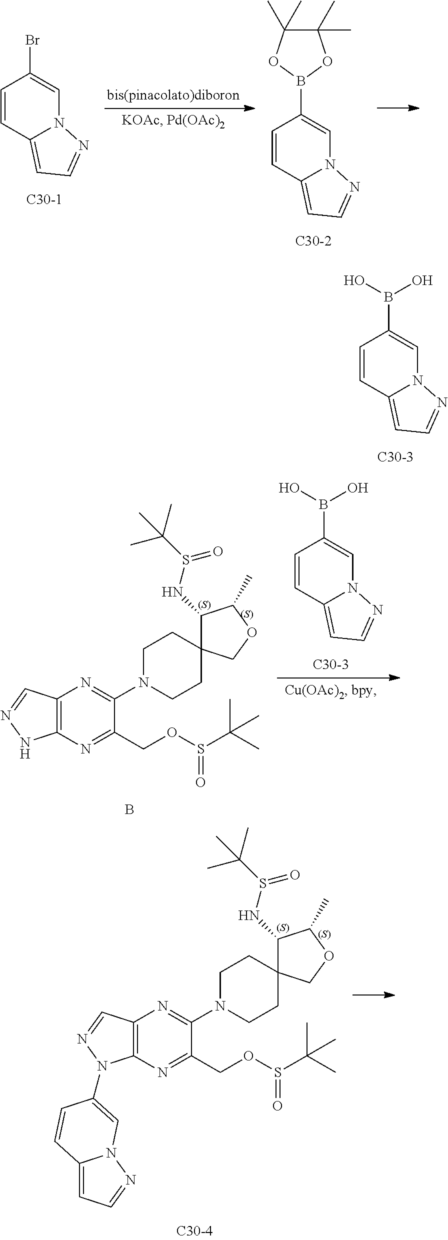

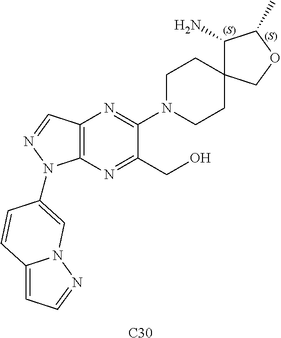

Compound C30 was synthesized with reference to the synthesis of compound C22 and the data are shown in Table I.

### EXAMPLE 26

The compound synthesized in the present invention:

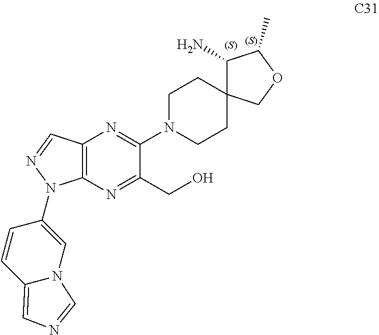

The synthetic route of compound C31 is as follows:

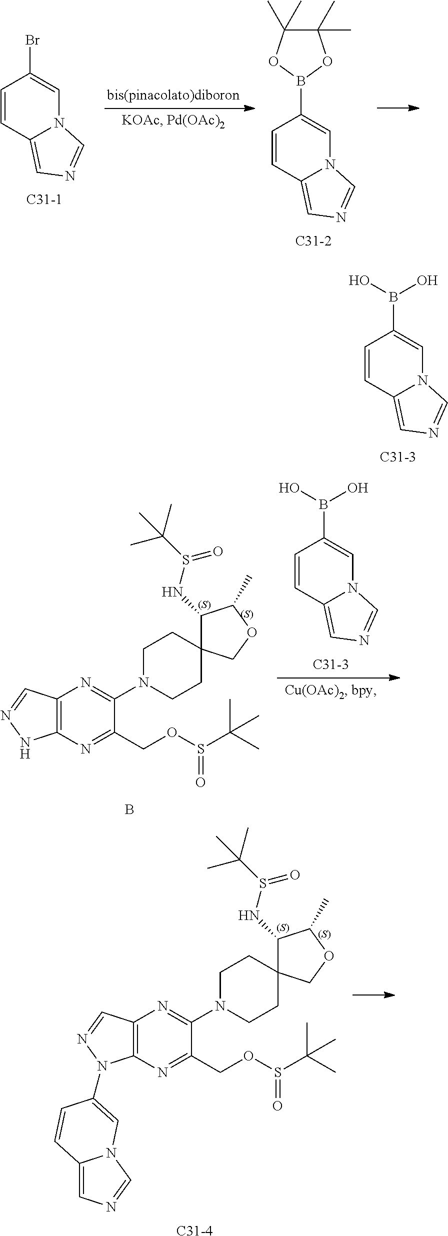

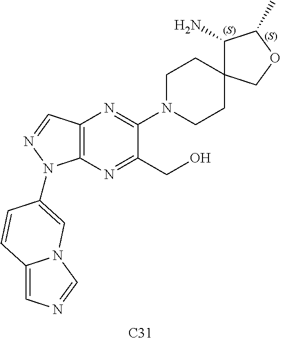

Compound C31 was synthesized with reference to the synthesis of compound C22 and the data are shown in Table I.

### EXAMPLE 27

The compound synthesized in the present invention:

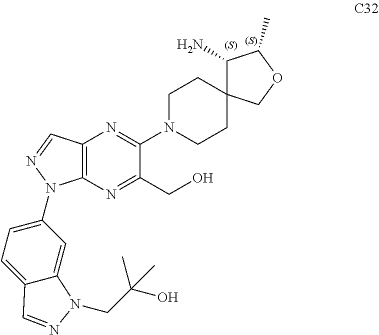

The synthetic route of compound C32 is as follows:

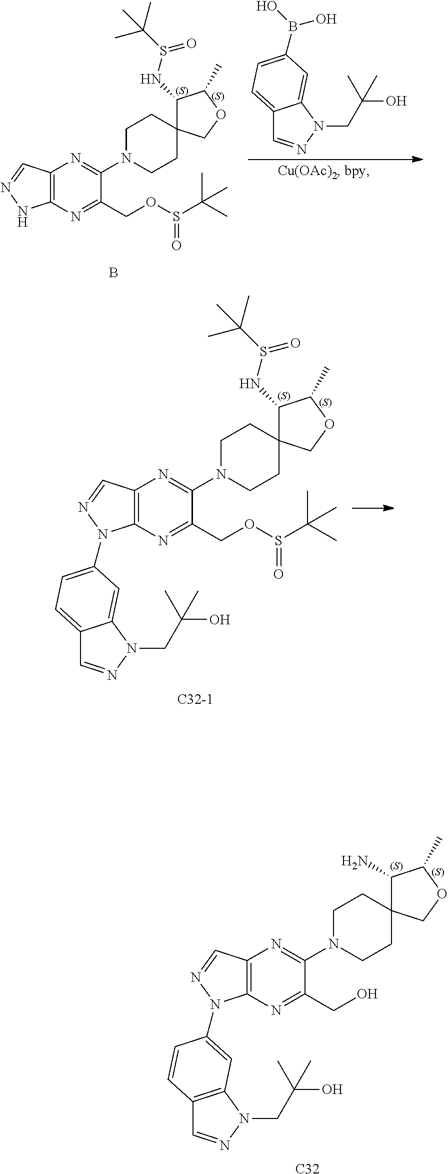

Compound C32 was synthesized with reference to the synthesis of compound C22 and the data are shown in Table I.

### EXAMPLE 28

The compound synthesized in the present invention:

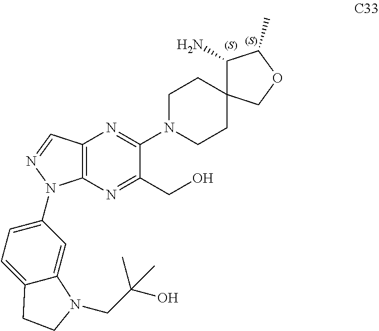

The synthetic route of compound C33 is as follows:

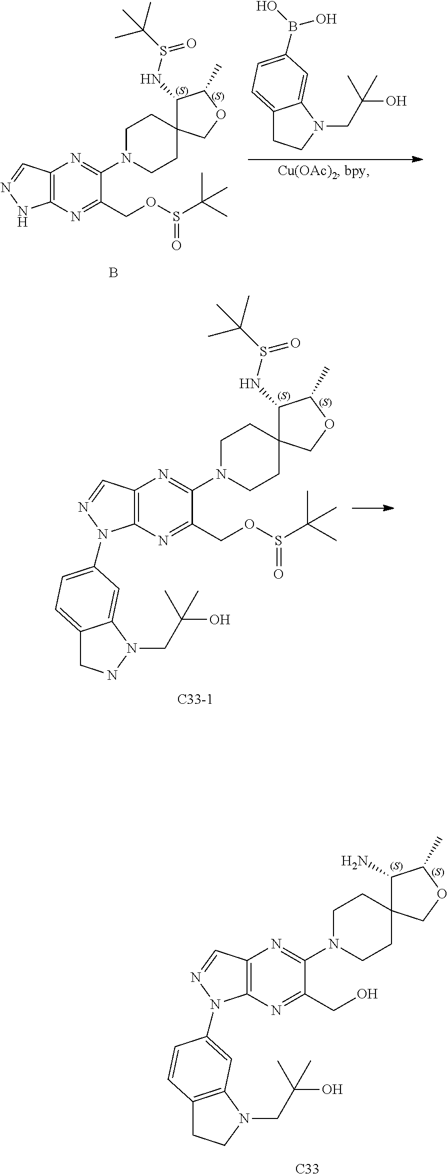

Compound C33 was synthesized with reference to the synthesis of compound C22 and the data are shown in Table I.

### EXAMPLE 29

The compound synthesized in the present invention:

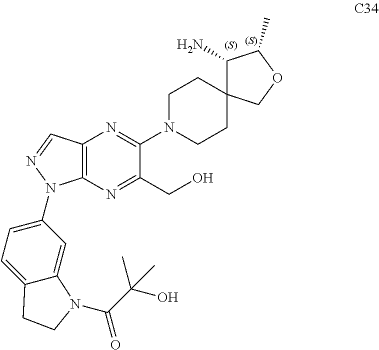

The synthetic route of compound C34 is as follows:

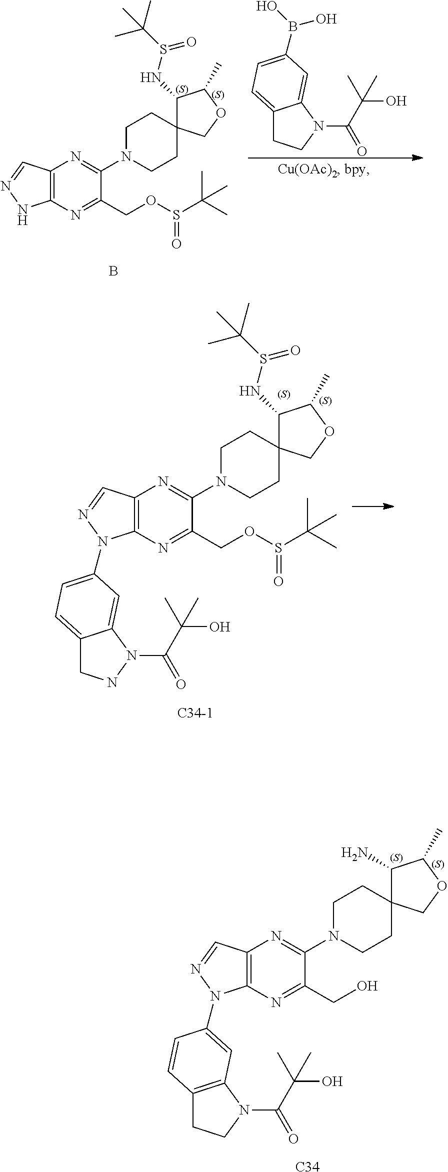

Compound C34 was synthesized with reference to the synthesis of compound C22 and the data are shown in Table I.

### EXAMPLE 30

The compound synthesized in the present invention:

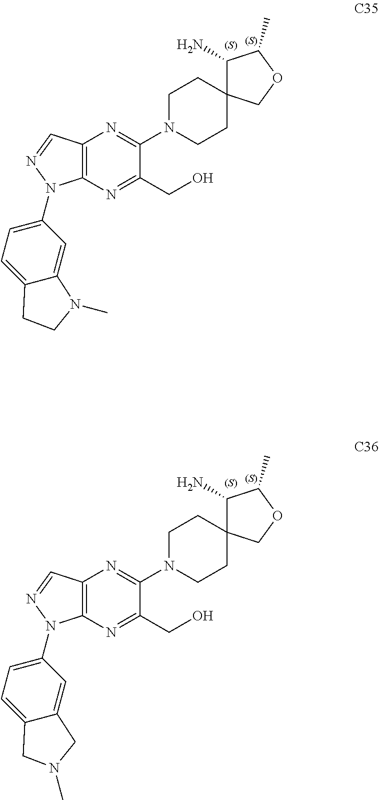

The synthetic route of compounds C35 and C36 are as follows:

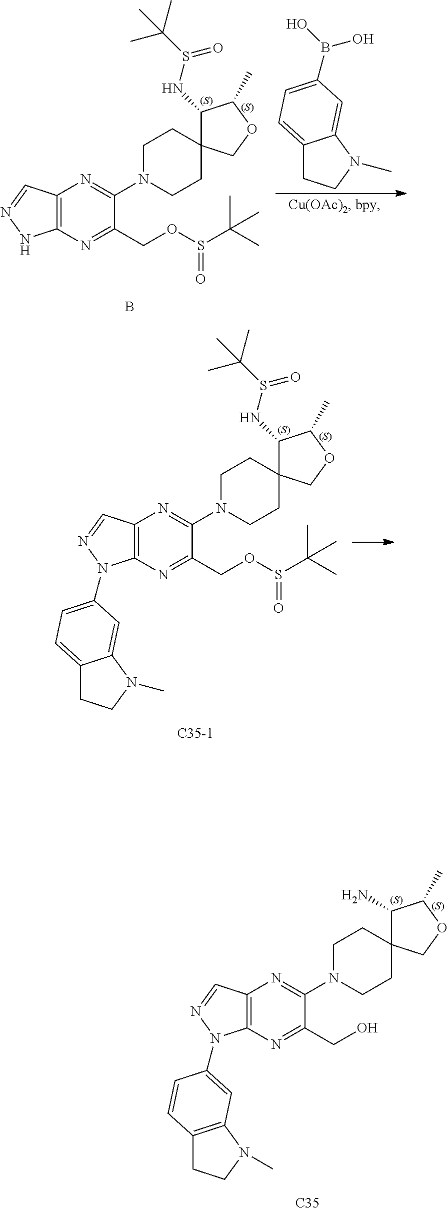

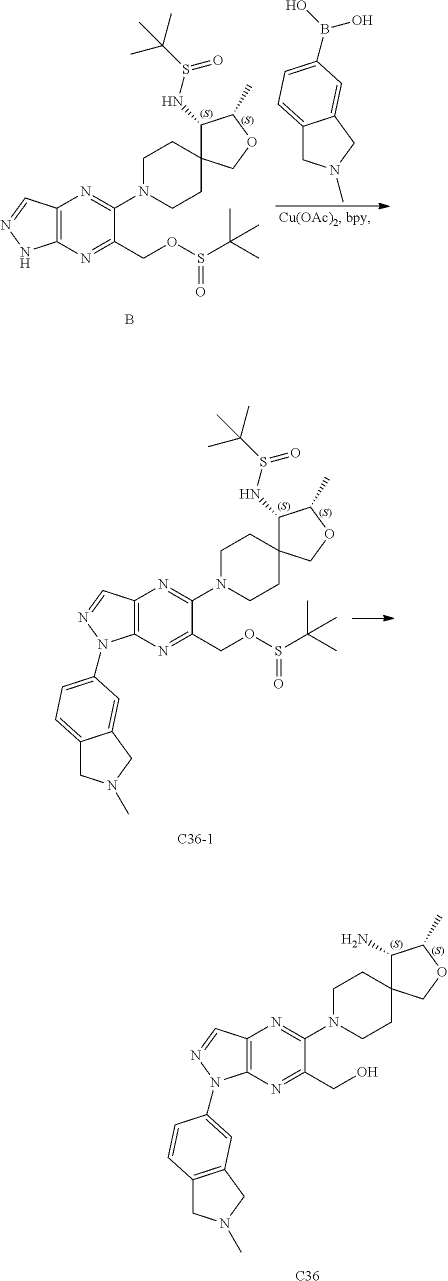

Compounds C35 and C36 were synthesized with reference to the synthesis of compound C22 and the data are shown in Table I.

TABLE 1

| Compound | Structure | Ms [M + H]⁺ | H¹NMR |
|---|---|---|---|
| C19 | 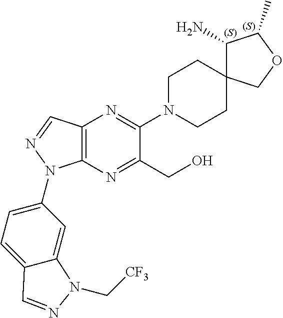 | 517.2 | (Methanol-d4) δ 8.61 (s, 1H), 8.28 (m, 2H), 8.17 (s, 1H), 7.94 (d, J = 8.8 Hz, 1H), 5.31 (q, J = 8.8 Hz, 2H), 4.93 (s, 2H), 4.31 (dt, J = 10.5, 5.2 Hz, 1H), 3.98 (d, J = 9.1 Hz, 1H), 3.87 (d, J = 9.1 Hz, 1H), 3.58 (d, J = 13.3 Hz, 1H), 3.54-3.45 (m, 2H), 3.14-2.91 (m, 2H), 2.06 (m, 2H), 1.95 (d, J = 12.8 Hz, 1H), 1.79 (d, J = 12.8 Hz, 1H), 1.34 (d, J = 6.5 Hz, 3H). |
| C20 | 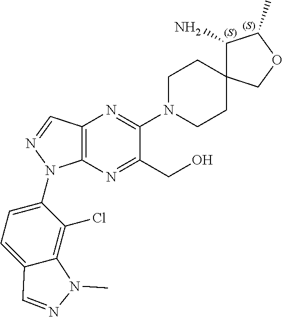 | 483.2 | |
| C21 | 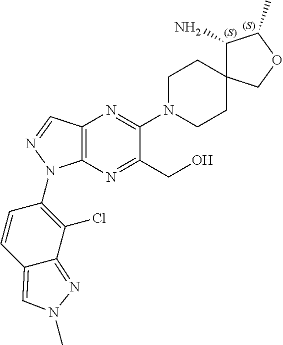 | 483.2 | |
| C22 | 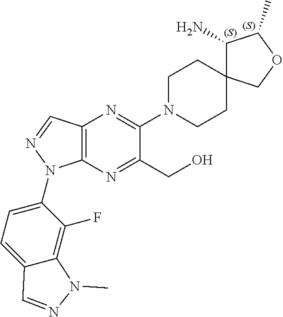 | 467.2 | (Methanol-d₄) δ 8.36 (s, 1H), 8.14 (d, J = 2.3 Hz, 1H), 7.72 (d, J = 8.5 Hz, 1H), 7.37 (dd, J = 8.5, 5.8 Hz, 1H), 4.84 (s, 2H), 4.36-4.30 (m, 1H), 4.28 (s, 3H), 3.98 (d, J = 9.1 Hz, 1H), 3.88 (d, J = 9.1 Hz, 1H), 3.62-3.43 (m, 3H), 3.11-2.96 (m, 2H), 2.05-2.02 (m, 2H), 1.95 (d, J = 13.2 Hz, 1H), 1.79 (d, J = 13.8 Hz, 1H), 1.34 (d, J = 6.5 Hz, 3H). |
| C23 | 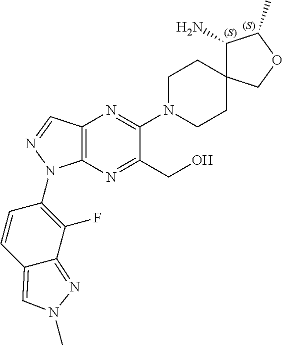 | 467.2 | (Methanol-d₄) δ 8.42 (d, J = 2.7 Hz, 1H), 8.34 (s, 1H), 7.68 (d, J = 8.8 Hz, 1H), 7.28 (dd, J = 8.8, 5.9 Hz, 1H), 4.82 (s, 2H), 4.35-4.31 (m, 1H), 4.30 (s, 3H), 3.98 (d, J = 9.1 Hz, 1H), 3.87 (d, J = 9.1 Hz, 1H), 3.62-3.45 (m, 3H), 3.10-2.98 (m, 2H), 2.08-2.02 (m, 2H), 1.95 (d, J = 13.5 Hz, 1H), 1.78 (d, J = 12.9 Hz, 1H), 1.33 (d, J = 6.5 Hz, 3H). |
| C24 | 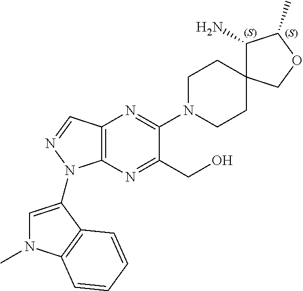 | 448.2 | |
| C25 | 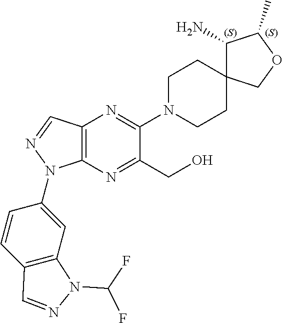 | 485.2 | |
| C26 | 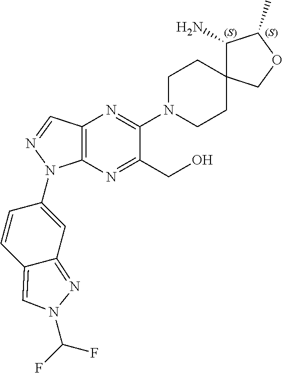 | 485.2 | |
| C27 | 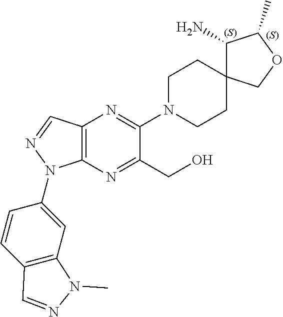 | 449.2 | |
| C28 | 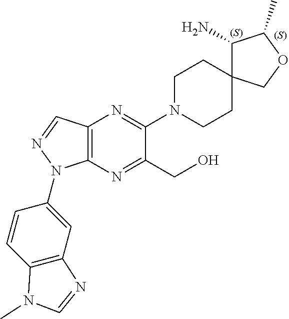 | 449.2 | |
| C29 | 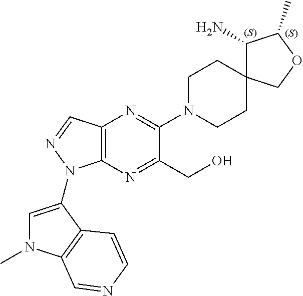 | 449.2 | |
| C30 | 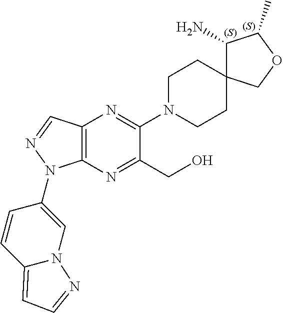 | 435.2 | |
| C31 | 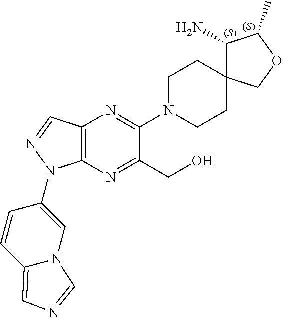 | 435.2 | |
| C32 | 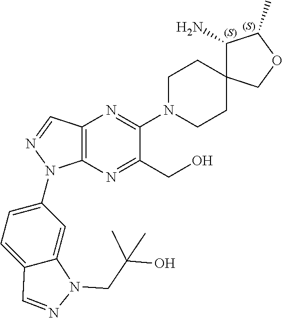 | 507.3 | |
| C33 | 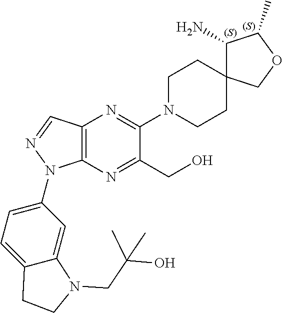 | 508.3 | |
| C34 | 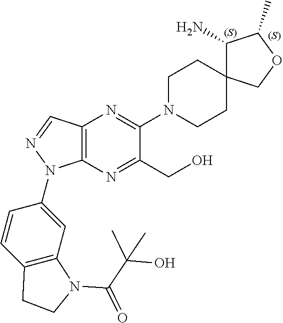 | 522.3 | |
| C35 | 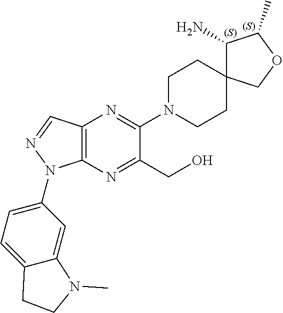 | 450.3 | |
| C36 | 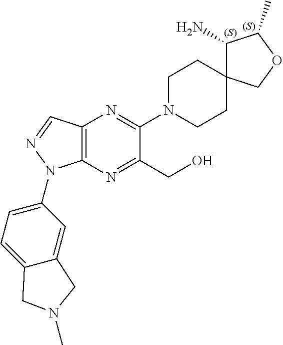 | 450.3 | |

The following experiments were performed to test the biological activity of some of the synthesized compounds.

### Example 1: In Vitro Evaluation

### 1. Experimental Reagents and Materials

a) Purified full-length SHP2 protein (Kamed Bio-Tianjin)
b) SHP2 activating peptide (BPS Bioscience).
c) DiFMUP.
d) Reaction buffer (120 mM HEPES pH 7.2, 200 mM NaCl, 1 mM EDTA, 0.002% Brij35), added with 0.04% BSA after autoclave, stored at 4° C.; diluted to 1× before use, and added with 2 mM DTT.

### 2. Experimental Steps

a) The 384-well plates were filled with 10 uL solution of compound to be tested (No. 2), 3-fold gradient dilution, totaling 8 concentrations;
b) The sample wells to be tested were added with 5 uL of Niv activating peptide (No. 4, 2 uM) and 5 uL of 4×full-length SHP2 protein (0.88 nM), control wells were free of inhibitor, and blank wells were free of SHP2 activating peptide and inhibitor;
c) 384-well plate was sealed, mixed well and incubated at room temperature for 1 hr;
d) After adding 5 uL 5×DiFMUP (125 uM), 384-well plate was sealed, mixed well and incubated at room temperature for 1 hr, and then detected by En Vision;

### 3. Data Analysis and Results

The inhibition rate was calculated as follows:

% Inhibition=[1−(RFUₛₐₘₚₗₑ−RFU_{blank})/(RFUₜₒₜₐₗ−RFU_{blank})]×100

Non-linear regression analysis was performed using Graphpad 8.0, and the IC₅₀ values for each compound were obtained by fitting a curve of enzyme activity with compound concentration through the equation Y=Bottom+(Top−Bottom)/(1+10\hat{ }((LogIC₅₀−X)*Hill Slope)). The IC50 of compounds inhibiting SHP2 enzyme activity is shown in Table II:

(In the table below, the following designations are used: <50nM=A; 50-500 nM=B; >500 nM=C).

TABLE II

| Compound | IC₅₀ (nM) |
|---|---|
| RMC-4630 | A |
| C4 | A |
| C5 | A |
| C6 | A |
| C11 | A |
| C12 | A |
| C13 | A |
| C14 | A |
| C15 | A |
| C16 | A |
| C17 | A |
| C18 | A |
| WO2021148010A1 | B |
| Example 5 | |

### Example 2: Evaluation of Compound Activity in MV4-11 Cells

The experimental procedure for bioactivity testing was as follows:

MV4-11 cells in logarithmic growth phase were taken, prepared into cell suspension, inoculated in 96-well plates at 160 uL/well with an inoculation density of 5000 cells/well, and cultured overnight in a 37° C. cell culture incubator. A 5× solution of the compound to be tested was prepared with 4-fold gradient dilution, and a total of 8 concentrations with double duplicate wells. The solution of the compound to be tested was added into 96-well plate at 40 uL/well, and the blank and control wells were added with corresponding volume of solvent, shaken and mixed well, and then incubated in 37° C. cell incubator for 72 hr, and the cell viability was detected by CTG method.

Equation (Sample−blank)/(control−blank)*100% was used to convert raw data to inhibition rate, and the IC₅₀ value could be obtained by curve fitting with four parameters (obtained with “log(inhibitor) vs. response—Variable slope” model in GraphPad Prism), and the results are shown in Table III. (In the table below, the following designations are used: <100 nM=A; 100-500 nM=B; >500 nM=C).

TABLE III

| Compound | IC₅₀ (nM) |
|---|---|
| C4 | A |
| C5 | A |
| C6 | A |
| C11 | A |
| C12 | A |
| C15 | A |
| C17 | A |
| WO2021148010A1 | B |
| Example 5 | |
| RMC-4630 | A |

From the above table, through in vitro bioactivity screening, using RMC-4630 as a control (structure formula

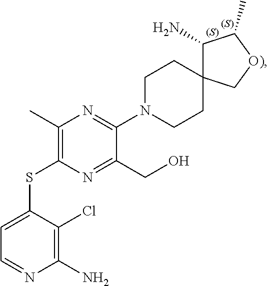

the compounds synthesized in the present application have good inhibitory ability against SHP2. It is expected to be further developed as a drug for use in the regulation of SHP2 activity or in the treatment of SHP2-related diseases. Example 5 of WO2021148010A1 has reported the structural formula as

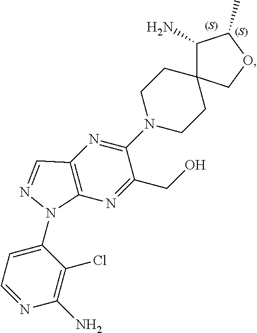

compared with this compound, it was found that the compound of the present application has better inhibitory ability against SHP2.

All documents referred to in the present invention are incorporated by reference herein as if each document is individually incorporated by reference. Further, it should be understood that upon reading the above teaching of the present invention, various alterations or modifications may be made to the present invention by those skilled in the art, and those equivalents also fall within the scope defined by the appended claims of the present application.

1. A compound of formula I′, or a pharmaceutically acceptable salt, a stereoisomer, a solvate or a prodrug thereof, 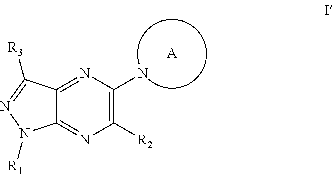
wherein,
R₁ is selected from the group consisting of bicyclic C6-C10 aryl, 6-10 membered heteroaryl containing 1-3 heteroatoms selected from N, O, and S, C6-C10 aryl heterocycloalkyl; any hydrogen atom on R₁ is optionally substituted by one or more of the following substituents: deuterium, hydroxyl, halogen, cyano, ═O, ester, acylamino, ketocarbonyl, amino, hydroxyl-substituted C1-C4 alkyl, —C(O)ORₐ, —NHC(O)Rₐ, —NHC(O)ORₐ, —C(O)(C1-C4 alkylene)OH, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C10 aryl, and 6-10 membered heteroaryl containing 1-3 heteroatoms selected from N, O and S; Rₐ is C1-C4 alkyl; the C6-C10 aryl heterocycloalkyl is —(C6-C10 aryl) fused (saturated or unsaturated 3-8 membered heterocycloalkyl containing 1-3 heteroatoms selected from N, O and S); R₁ is a bicyclic and fused-ring structure;
R₂ is selected from the group consisting of H, deuterium, amino, cyano, halogen, hydroxyl, methyl, CH₂OH, CH(CH₃)OH, C(CH₃)₂OH, halomethyl, deuterated methyl, CONH₂, CF₂OH, NHSO₂Me, and CH₂NHSO₂Me;
R₃ is selected from the group consisting of hydrogen, deuterium, hydroxyl, amino, cyano, halogen, methyl, deuteromethyl, and halomethyl;
ring A is selected from the group consisting of: monocyclic or bicyclic 3-11 membered heterocycloalkyl containing 1-3 heteroatoms selected from N, O and S, 6-10 membered heteroaryl containing 1-3 heteroatoms selected from N, O and S, -(3-8 membered heterocycloalkylene containing 1-3 heteroatoms selected from N, O and S)-(3-8 membered heterocycloalkyl containing 1-3 heteroatoms selected from N, O and S), 4-8 membered bridged heterocycloalkyl containing 1-3 heteroatoms selected from N, O and S;
any hydrogen atom on ring A is unsubstituted or monosubstituted, disubstituted or trisubstituted by the following substituents: (CH₂),NHR′₁, (CH₂),CONH₂, (CH₂)ₙCF₂H, (CH₂)ₙCF₃, (CH₂)ₙOH, ═O, C1-C6 alkyl, halogen, amino, hydroxyl, —N—(C1-C6 alkyl), —(C1-C6 alkylene)—NH₂, wherein the hydrogen on the alkyl is unsubstituted or monosubstituted or disubstituted by OR′₁;
R′₁ is selected from the group consisting of: H, C1-C4 alkyl, and hydroxyl-substituted C1-C4 alkyl; and
n is selected from the group consisting of 0, 1, 2 and 3.

2. The compound according to claim 1, or a pharmaceutically acceptable salt, a stereoisomer, a solvate or a prodrug thereof, wherein
R₁ is a ring B-fused-ring C, wherein,
ring B and ring C are each independently selected from the group consisting of: C5-C6 aryl, 5-6 membered heteroaryl containing 1-3 heteroatoms selected from N, O and S, C5-C6 cycloalkyl, saturated 5-6 membered heterocycloalkyl containing 1-3 heteroatoms selected from N, O and S; and
any hydrogen atom on R₁ is optionally substituted by one or more of the following substituents: deuterium, hydroxyl, halogen, cyano, ═O, amino, hydroxyl-substituted C1-C4 alkyl, C1-C6 alkyl, C1-C6 haloalkyl, C1-C6 thioalkyl, C1-C6 alkoxy, C3-C6 cycloalkyl, C1-C6 alkylamino, C6-C10 aryl, 6-10 membered heteroaryl containing 1-3 heteroatoms selected from N, O and S, and —C(O)C(CH₃)₂OH.

3. The compound according to claim 2, or a pharmaceutically acceptable salt, a stereoisomer, a solvate or a prodrug thereof, wherein
R₁ is selected from the group consisting of: 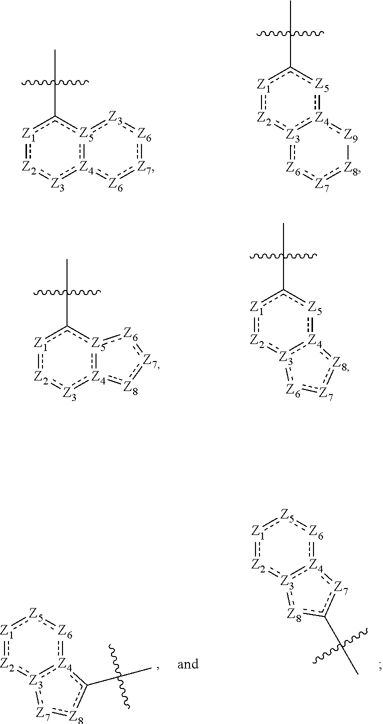
Z₁, Z₂, Z₃, Z₄, Z₅, Z₆, Z₇, Z₈, and Z₉ are each independently selected from the group consisting of N, O, S, C, C(R₄)ₘ and NR₄;
each R₄ is independently selected from the group consisting of: hydrogen, deuterium, hydroxyl, halogen, cyano, ═O, amino, hydroxyl-substituted C1-C4 alkyl, C1-C6 alkyl, C1-C6 haloalkyl, C1-C6 thioalkyl, C1-C6 alkoxy, C3-C6 cycloalkyl, C1-C6 alkylamino, C6-C10 aryl, 6-10 membered heteroaryl containing 1-3 heteroatoms selected from N, O and S, and —COC(CH₃)₂OH; [symbol]
is a single or double bond; and
each m is independently selected from the group consisting of 1 and 2.

4. The compound according to claim 3, or a pharmaceutically acceptable salt, a stereoisomer, a solvate or a prodrug thereof, wherein R₁ is selected from the group consisting of:

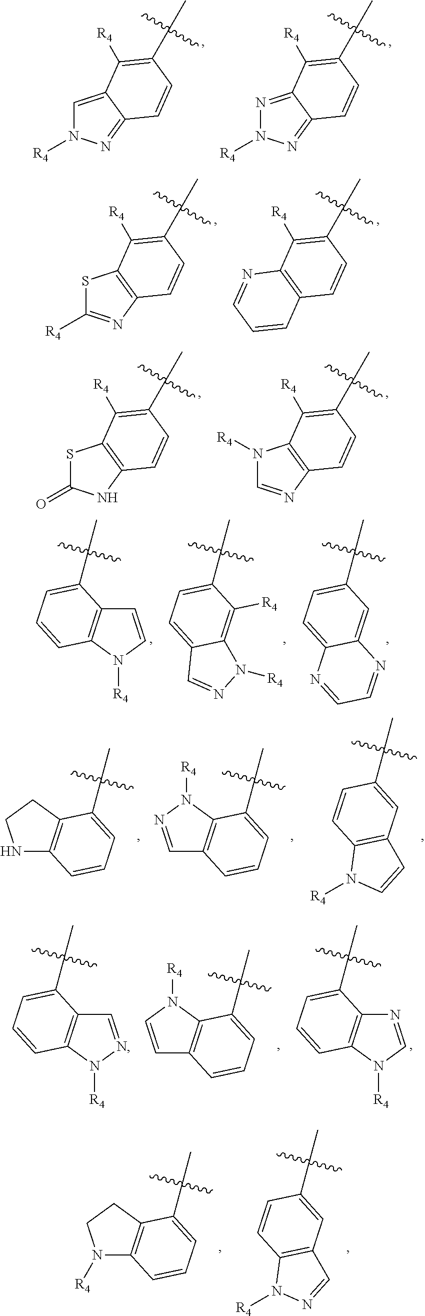

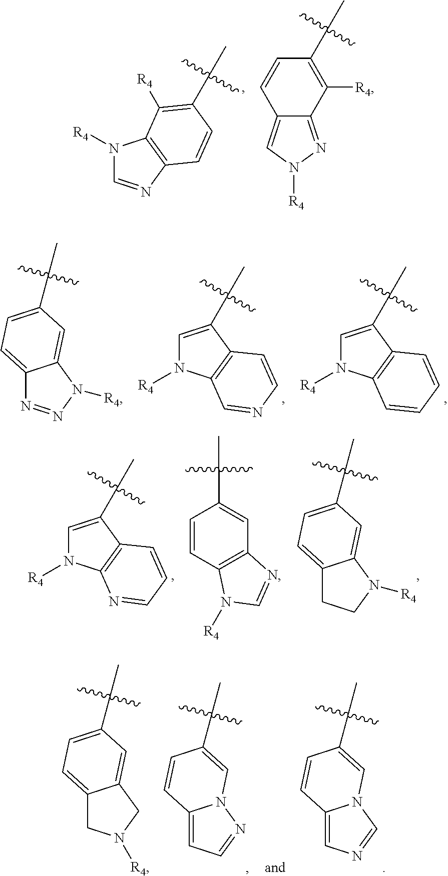

5. The compound according to claim 1, or a pharmaceutically acceptable salt, a stereoisomer, a solvate or a prodrug thereof, wherein ring A is

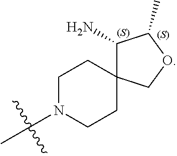

6. The compound according to claim 1, or a pharmaceutically acceptable salt, a stereoisomer, a solvate or a prodrug thereof, wherein the compound is selected from the group consisting of:

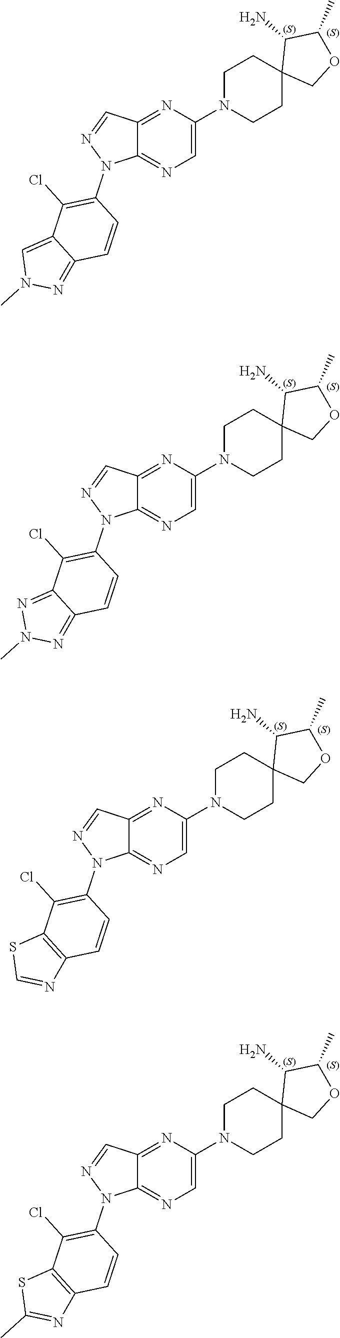

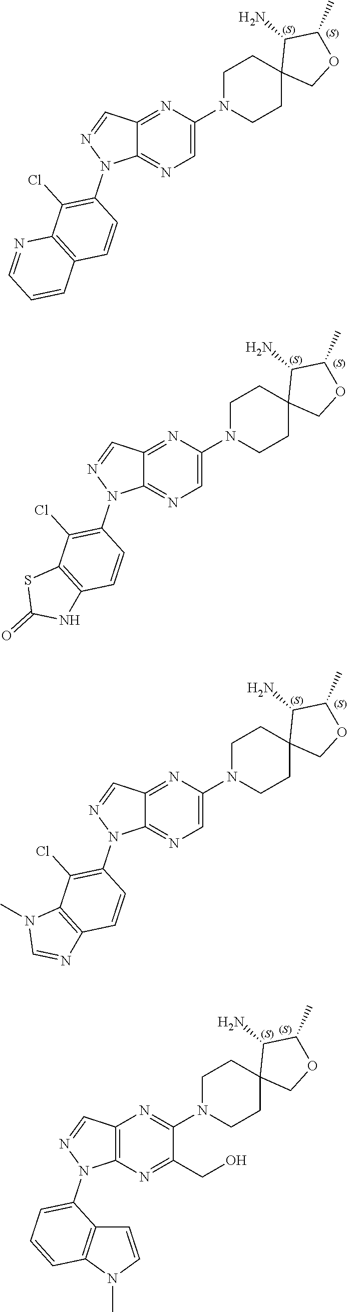

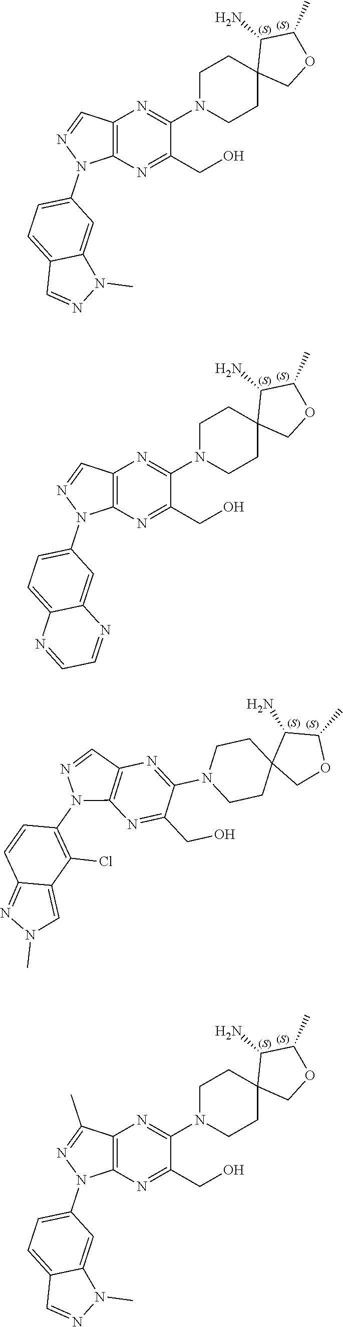

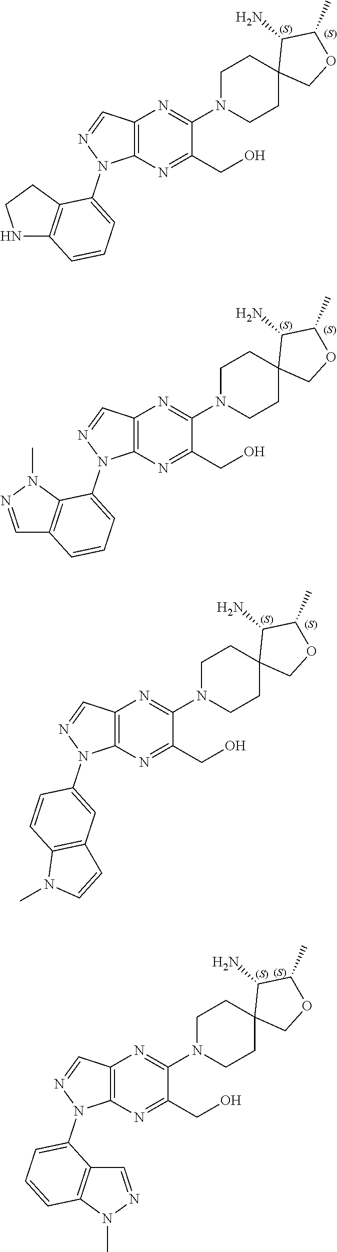

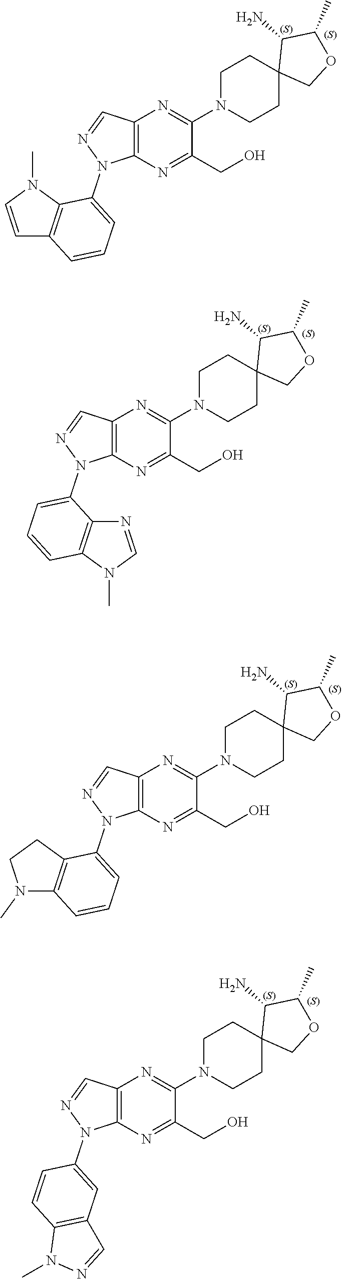

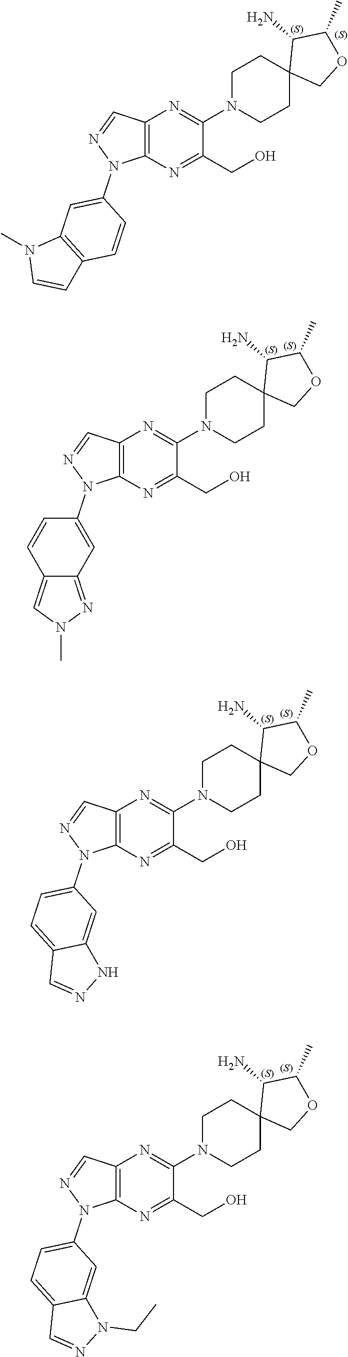

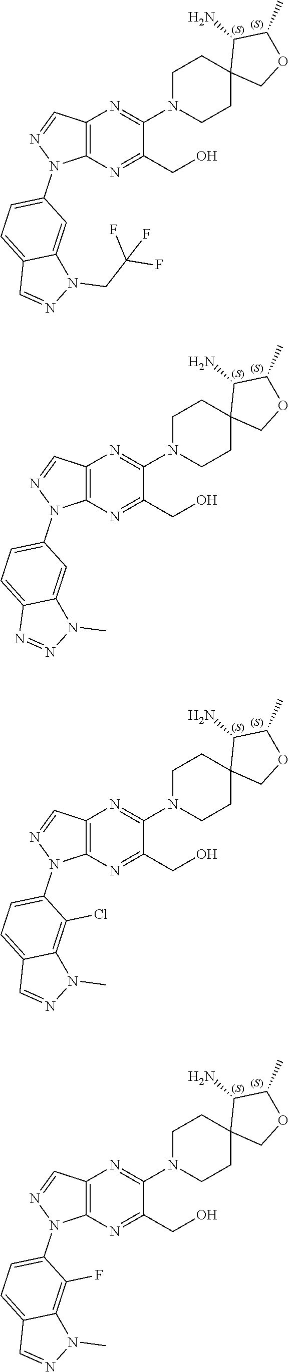

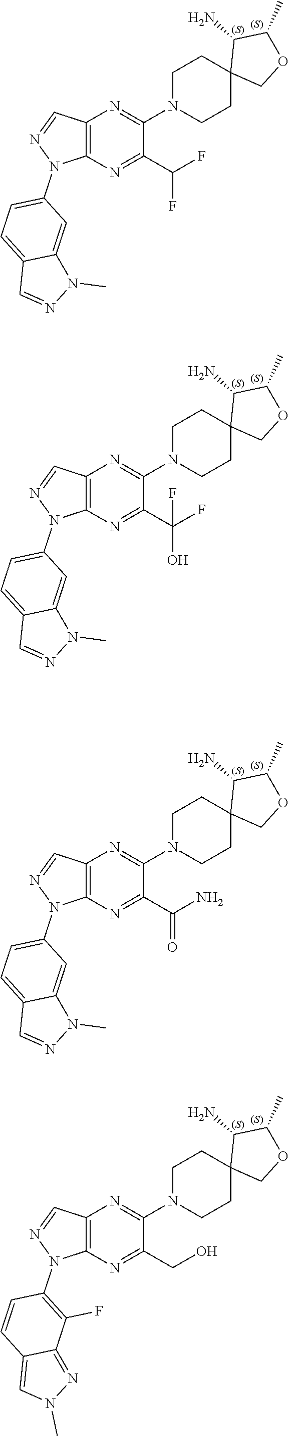

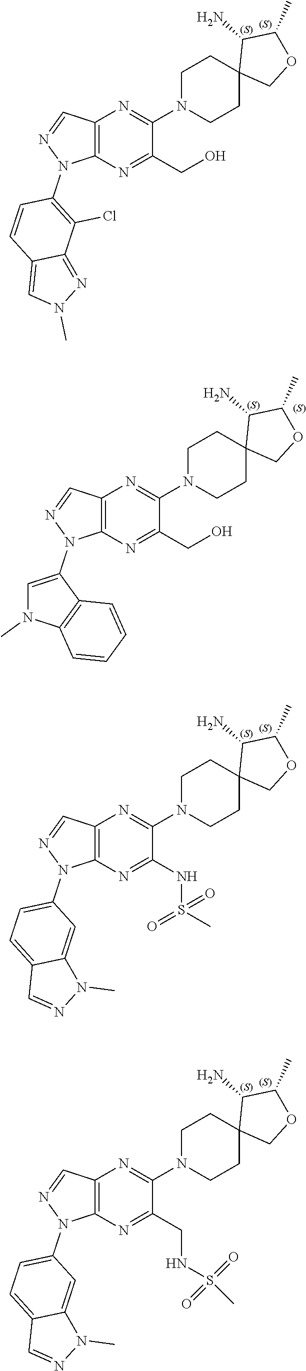

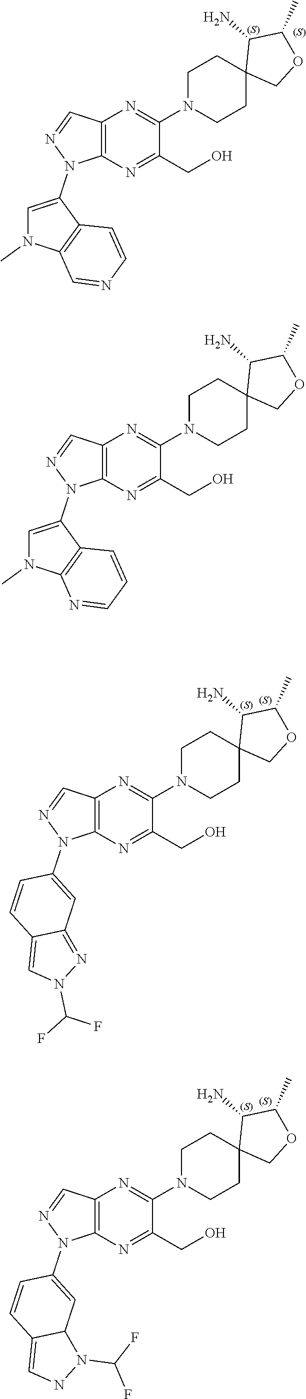

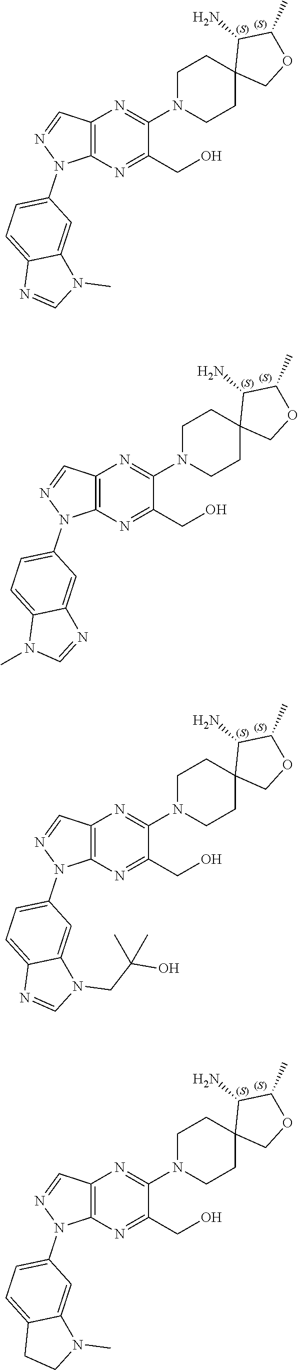

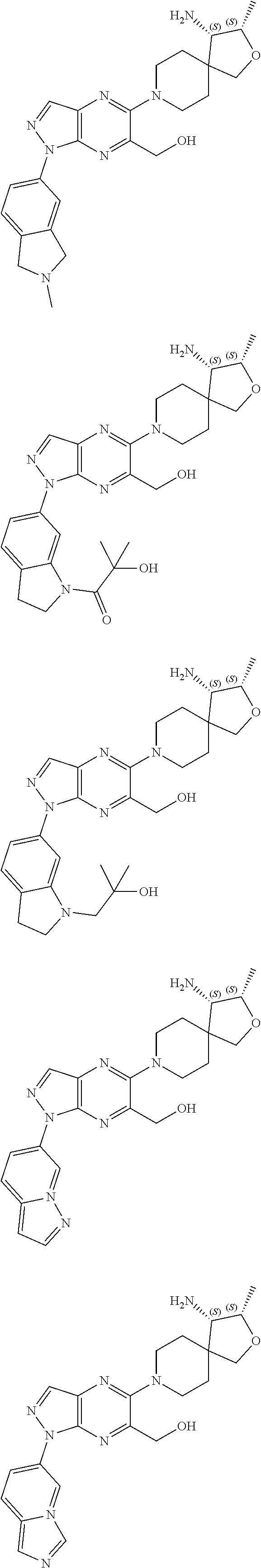

7. A pharmaceutical composition, comprising a pharmaceutically acceptable carrier and one or more safe and effective amounts of the compound according to claim 1, or a pharmaceutically acceptable salt, a stereoisomer, a solvate or a prodrug thereof.

8. A use of the pharmaceutical composition according to claim 7 in the preparation of a medicament used as a SHP2 inhibitor.

9. A use of the pharmaceutical composition according to claim 7 in the preparation of a medicament for regulating SHP2 activity or treating SHP2-related diseases.

10. The use of claim 9, wherein the SHP2-related disease is selected from the group consisting of Noonan syndrome, Leopard syndrome, juvenile myelomonocytic leukemia, acute myeloid leukemia, neuroblastoma, melanoma, breast cancer, esophageal cancer, lung cancer, gastric cancer, head cancer, anaplastic large cell lymphoma, neuroblastoma, glioblastoma, squamous cell carcinoma of the head and neck, colon cancer, and liver cancer.
